(19) United States
(12) Patent Application Publication
(10) Pub. No.: US 2025/0179550 A1
(43) Pub. Date: Jun. 5, 2025

(54) Methods to imbue polymeric materials with new structures and functions

(71) Applicant: The Regents of the University of California, Oakland, CA (US)

(72) Inventors: Alanna Schepartz Shrader, Berkeley, CA (US); Joshua A. Walker, Berkeley, CA (US)

(73) Assignee: The Regents of the University of California, Oakland, CA (US)

(21) Appl. No.: 18/747,424

(22) Filed: Jun. 18, 2024

Related U.S. Application Data
(63) Continuation of application No. PCT/US22/80459, filed on Nov. 23, 2022.
(60) Provisional application No. 63/295,885, filed on Jan. 1, 2022.

(51) Int. Cl. C12P 21/02; C07K 1/113; C12N 9/88

(52) U.S. Cl. CPC C12P 21/02; C07K 1/113; C12N 9/88; C12Y 402/01

(57) ABSTRACT

Polymer engineering is achieved by deploying a constitutively active dehydratase enzyme of RiPP biosynthesis to accept a substrate containing a non-α-amino acid monomer at a position flanking the reaction site.

### CROSS-REFERENCES TO RELATED APPLICATIONS

This application is a continuation of PCT/US22/80459, filed Nov. 23, 2022, which claims priority to U.S. Provisional Application No. 63/295,885; filed Jan. 1, 2022 the disclosures of which are hereby incorporated by reference in its entirety for all purposes.

### GOVERNMENT SUPPORT CLAUSE

This invention was made with government support under the National Science Foundation, grant numbers 2002182 and 2021739. The government has certain rights in the invention.

### REFERENCE TO A SEQUENCE LISTING

A Sequence Listing in XML format is incorporated by reference into the specification. The name of the XML file containing the Sequence Listing is B21-125-2US.xml. The XML file is 62,097 bytes and was created on Jun. 17, 2024.

### INTRODUCTION

Ribosomally synthesized and post-translationally modified peptides (RiPPs) are a unique class of natural products (https://doi.org/10.1039/D0NP00027B). Their synthesis begins with a ribosomally translated precursor peptide that is elaborated further by enzymes that convert one or more traditional peptide linkages into those that contain heterocycles, D-amino acids, intrachain crosslinks, and backbone and side chain modifications (FIG. 1). Ribosomally translated precursor peptides contain two functional units—an N-terminal leader peptide that recruits one or more modifying enzymes and a C-terminal core peptide on which the modifying enzyme(s) act (FIG. 2) DOI: 10.1039/c2np20085f. Many RiPP natural products have been identified and include the FDA-approved analgesic ziconotide (DOI: 10.1002/cmdc.201200513) as well as compounds with antibiotic, antiviral, and antitumor properties. A common RiPP modification is installed by a family of enzymes known as heterocyclases (FIG. 3). Heterocyclases catalyze the cyclization of a serine, threonine, or cysteine side chains to form an azoline heterocycle, which is dehydrogenated to yield an unsaturated azole heterocycle (FIG. 3). Azole heterocycles appear quite often in small molecule drugs. https://doi.org/10.1021/jm51100b.

### RELEVANT LITERATURE

Goto and Suga developed in vitro translation systems for the rapid prototyping of heterocyclase substrates (DOI: 10.1016/j.chembiol.2014.04.008; https://doi.org/10.1246/c1.160562). These systems were applied to study the influence of flanking sequences on the activity of the heterocyclase enzyme PatD. This work demonstrated that PatD activity is insensitive to polypeptide sequences flanking the core peptide. However, PatD was found to be sensitive to deletions and point mutations within the leader peptide.

Bowers, Goto, Hicks, and Suga combined in vitro translation, chimeric leader peptides, and unnatural amino acids (UAAs) to create a semi-synthetic approach to synthesizing thiopeptides such as thiocillin and lactazole (https://doi.org/10.1021/jacs.8b11521). This platform was used to investigate the influence of core peptide point mutations as well as deletions and insertions within the leader peptide on the activity of the heterocyclase enzyme LynD. Generally, core peptide point mutations were tolerated, but leader peptide modifications impacted yield. All changes introduced into the core sequence were natural a-amino acids.

Recently, Goto and Suga utilized in vitro translation and flexizyme-mediated tRNA charging of UAAs to further explore the substrate scope of PatD (https://doi.org/10.1002/cbic.201900521; https://doi.org/10.1002/anie.201910894). These works demonstrated that PatD, which natively accepts sulfhydryl and hydroxyl nucleophiles, also accepts amino nucleophiles. PatD also tolerated methylene insertion within the side chain, which gave rise to 6-membered heterocyclic products. Further, modifications to the side chain substituent of cysteine and threonine gave rise to 5-membered heterocycles with non-natural substituents.

Onaka, and Suga extended their work on PatD substrate tolerance by utilizing in vitro translation to recapitulate the biosynthesis of lactazole A (doi: https://doi.org/10.1101/807206). This work demonstrated that the heterocyclase LazD tolerates expansion and contraction of the macrocyclic loop of lactazole A. Further, UAAs and β-amino acids were tolerated within the macrocyclic loop. However, these unnatural residues were located at positions distant from the residues targeted for heterocyclization by LazD.

Other researchers have studied the substrate tolerance of native heterocyclases. Early work from Jaspars, Naismith, and Smith explored the substrate tolerance of PatD and TruD [68]. PatD and TruD were found to cyclize selenocysteine residues to yield selenazolines. In a later work, this in vitro synthesis scheme was expanded to include the heterocyclases LynD, MicD, and TenD [69]. A mutational study established that heterocyclases tolerate a variety of α-amino acid substitutions within the core peptide sequence.

Jaspars and Naismith developed a set of constitutively active heterocycases in which the leader sequence is fused directly to the enzyme, such as LynD and MicD. These constructions are referred to as “fusions” (such as MicD fusion) (DOI: 10.1038/nchembio.1841). In line with previous studies of TruD (DOI: 10.1002/anie.201306302) and PatD (DOI: 10.1016/j.chembiol.2014.04.008) this work found that native LynD retains limited activity towards leaderless core peptides. Fusion of the leader peptide to the N-terminus of LynD led to the creation of a constitutively activated variant, LynD_fusion. LynD_fusion processed leaderless core peptides, but point mutations impacted the efficiency of heterocyclization. Recently, this concept was used to constitutively activate the heterocyclase MicD, and study the processivity of heterocyclases (https://doi.org/10.1021/acs.biochem.9b00084).

Migaud, Naismith and Westwood have since explored the substrate tolerance of constitutively activated heterocyclases (https://doi.org/10.1002/open.201600134; https://doi.org/10.1002/cbic.201500494). These efforts demonstrated that LynD_fusion tolerates the insertion of a 4-unit polyethylene glycol spacer or azidoalanine within the core peptide. However, much like work from Goto, Onaka, and Suga, these modifications were made at positions distant from the residue heterocyclized by LynD_fusion.

Gaps in Knowledge with Respect to Heterocyclase Substrate Scope

Existing approaches to the synthesis of novel compositions of matter using RiPP heterocyclases have notable limitations. Studies have demonstrated that heterocyclases process core peptides containing multiple proteinogenic α-amino acids, but studies of substrates containing non-natural monomers have been severely limited. No study has reported heterocyclase activity for a substrate containing a non-α-amino acid residue at position +1 or −1, or incorporated a non-natural backbone monomer at the +1 site. Reliance on the paradigm of leader peptide-directed modification has provided valuable insight into the biochemistry of native heterocyclases, but makes it difficult to apply the chemistry to valuable substrates such as antibodies, cytokines, replacement enzymes, or other therapeutic proteins. No study has modified the activity, processivity, or selectivity of native heterocyclases. These limitations restrict the structural diversity of compounds accessible via heterocyclase biosynthesis.

### SUMMARY OF THE INVENTION

We demonstrate that constitutively active dehydratase enzymes involved in RiPP biosynthesis can accept substrates containing multiple, diverse, non-α-amino acid monomers at positions flanking the reaction site. We demonstrate that this reactivity can be used to engineer therapeutic proteins that express at higher levels, resist degradation, alter immune reactions, and encode functions that add additional value, such as but not limited to targeting the protein to distinct cells or tissues.

The invention expands the chemistry of the heterocyclase MicD in multiple unique ways. We reveal that multiple, structurally diverse aromatic rings are tolerated at the +1 position that precedes the site of cyclization. We reveal that multiple, structurally diverse beta-β-amino acids are also tolerated at the +1 site. We reveal that aramid monomers are tolerated at the −1 site. Finally, we reveal that MicD can introduce an azoline heterocycle into one or more loops of GFP to generate proteins with altered and improved properties.

### The Invention Provides Methods and Compositions

In an aspect the invention provides a method of polymer engineering: deploying a constitutively active dehydratase enzyme of RiPP biosynthesis to accept a substrate containing a non-α-amino acid monomer at a position flanking the reaction site.

In embodiments:
the polymer is or comprises an amino acid polymer (e.g. peptide, polypeptide or protein), or hybrid polymer or any polymer compatible and operable with the disclosed constitutively active dehydratase enzyme of RiPP biosynthesis to accept a substrate containing a non-α-amino acid monomer at a position flanking the reaction site;
the polymer is or comprises an antibody, cytokine, replacement enzyme, or therapeutic protein;
the method imbues a polymeric material with new structures and/or functions;
the method introduces a non-native backbone modification to expand and/or improve protein function;
the method is used to engineer a therapeutic protein to express at higher levels (ease or improve production), resist degradation, improve thermal and proteolytic stability, alter immunogenicity, antigenicity or immune reactivity, or encode a function that adds additional value, such as but not limited to targeting the protein to distinct cells or tissues;
the enzyme catalyzes formation of an azole (e.g. oxazole or thiazole) ring;
the enzyme is selected from heterocyclases MicD, PatD, and LynD.
the method provides a protein therapeutic fused to a RiPP natural product, synthesized in a single step, without a separate chemical bio-conjugation step (which requires significant optimization), without sequential or separate purification steps, and/or in situ.
multiple, structurally diverse aromatic rings are tolerated at the +1 position that precedes the site of cyclization, e.g. Benzoic acid, tetrafluoro-benzoic acid, 2-amino benzoic acid, 2-amino-5-methoxy benzoic acid, 2-aminopyridyl, and coumarin;
multiple, structurally diverse beta-β-amino acids are tolerated at the +1 site, e.g. beta-3-isoleucine;
aramid monomers are tolerated at the −1 site; and/or
the enzyme (e.g. MicD) introduces an azoline heterocycle into one or more loops of a target protein (e.g. GFP) to generate proteins with altered and improved properties.

The invention encompasses all combinations of the particular embodiments recited herein, as if each combination had been laboriously recited.

### BRIEF DESCRIPTION OF THE DRAWINGS

FIG. 1. Examples of post-translational modifications installed by enzymes associated with RiPP biosynthesis.

FIG. 2. Overall topology of a RiPP substrate. The modification enzyme (shown in red) is recruited to (and activated by) the leader sequence to act on the peptide core at one or more positions.

FIG. 3. General reaction catalyzed by dehydratase enzymes such as MicD, PatD, and LynD. In some cases, a second enzyme performs a second dehydration step to convert the azoline into an azole.

FIG. 4. The chemical reaction catalyzed by dehydratase enzymes such as MicD. The target site contains the amino acid that participates directly in the dehydration reaction. This site is flanked on the N-terminal side (the +1 site) as well as the C-terminal side (the −1 site).

FIG. 5A-B. MicD accepts diverse benzoic acid monomers at the +1 site. Benzoic acid; tetrafluoro-benzoic acid; 2-amino benzoic acid; 2-amino-5-methoxy benzoic acid; 2-aminopyridyl; and coumarin.

FIG. 6. Conversion of a peptide containing a beta-3-isoleucine residue at position +1 into the azoline product using MicD-fusion.

FIG. 7. Conversion of a peptide containing a non-α-amino acid substrate at the −1 position using MicD-fusion.

FIG. 8A-F. Overview of cyclodehydratase/dehydrogenase chemistry. (A) Scheme illustrating the natural conversion of a serine, threonine, or cysteine-containing polypeptide into a oxazoline or thiazoline-containing product through the action of MicD26 and subsequent dehydrogenation into an oxazole or thiazole through the action of ArtGox.29 (B) Natural substrates for MicD37 and ArtGox38 consist of a core sequence that includes the reaction site and an extended upstream leader sequence. (C) Fusion of the leader sequence to the N-terminus of MicD generates a constitutively activated enzyme MicD-F that processes leaderless substrates.26 (D) This invention: MicD-F and ArtGox accept polypeptide substrates containing diverse non-α-amino acid monomers, including aramids, at the +1 and −1 site. MicD-F and ArtGox can also install thiazoline and thiazole linkages within globular proteins such as (E) mCherry and (F) Rop.

FIG. 9A-D. MicD-F and ArtGox tolerate diverse non-proteinogenic, non-α-amino acid monomers at the +1 site. (A) Scheme illustrating the conditions used for the reaction of ArtGox and/or MicD-F with substrates containing non-α-amino acid monomers N-terminal to the reaction site (+1 site). (B) Yields of thiazoline 2(a-i) and thiazole (3(a-i)) products obtained for substrates containing non-α-amino acid monomers at +1 site. Extracted ion chromatograms illustrating the products of (C) MicD-F and (D) MicD-F+ArtGox-catalyzed reactions.

FIG. 10A-C. MicD-F and ArtGox tolerate diverse non-proteinogenic, non-α-amino acid monomers at the −1 site. (A) MicD-F and ArtGox reactions of substrates with non-α-amino acid monomers immediately C-terminal to the site of cyclization. (B) Yields of thiazoline 5(a-i) and thiazole (6(a-i)) products obtained for polypeptide substrates with non-α-amino acid monomers at position −1, immediately C-terminal to the reaction site. Extracted ion chromatograms illustrating the products of (C) MicD-F and ArtGox-catalyzed reactions.

FIG. 11A-D. (A) mCherry variants evaluated as substrates for ArtGox and/or MicD-F. Each variant contains the sequence MCAYDG inserted following the residue shown. LC/MS analysis of mCherryC+ both (B) before and (C) after reaction with 50 mol % MicD-F or (D) 50 mol % MicD-F and 80 mol % ArtGox. Data reported are normalized counts from deconvoluted mass spectra. Asterisk indicates the molecular weight of the parent protein without a mature chromophore (+22 Da).

FIG. 12-A-F. MicD-F and ArtGox act in tandem to install thiazoline and thiazole backbones within globular proteins. (A) Cartoons illustrating the sequences of RopC, RopM, and RopN and the conditions used for MicD-F-catalyzed cyclodehydration (left arrow) or tandem cyclodehydration/dehydrogenation catalyzed by MicD-F and ArtGox (right arrow). (B) Wavelength-dependent circular dichroism spectra of RopC, RopM, and RopN at [monomer]=20 μM in 10 mM phosphate, 100 mM NaCl, 150 μM TCEP, pH 7.0 and 25° C. (C) LC/MS analysis of the reaction of RopC, RopN, and RopM with MicD-F under the conditions shown in panel (A) above. The characteristic loss of 18 mass units upon cyclodehydration is evident for both RopC and RopM; the reaction of RopM was incomplete under these conditions. (D) Treatment of RopC with MicD-F and ArtGox under the conditions shown in panel (A) above led to clean conversion into the corresponding thiazole (RopC-Z). (E) The wavelength-dependent CD spectra of RopC-U and RopC-Z compared to RopC; these data are not corrected for contributions due to the thiazoline or thiazole linkage. (F) The melting temperatures of RopC, RopC-U, and RopC-Z (after refolding) are almost identical.

FIG. 13A-G. Conformational effects of thiazoline and thiazole formation. (A) The open chain and cyclized analogs Ac-AACA-NH₂ were examined. Initial conformational searches were conducted using MacroModel (OPLS4 force field). All species within 4 kcal/mol of the global minimum were geometry optimized using DFT (B3LYP/6-31G**, SM8 solvent model) and re-ranked. (B-E) Lowest energy conformers are superimposed for different energy cutoff values. (F) A rigid 6-bond motif (green) describes all identified conformers within 2.72 kcal/mol of the global minimum for the thiazoline. (G) A similar 7-bond motif describes the thiazole conformers. k/m=kcal/mol. For images of all structures within 4.08 kcal/mol of each global minimum, see Supplementary Materials.

FIG. 14A-C. Characterization of MicD-F and ArtGox purified via Ni-NTA affinity chromatography. (A) SDS-PAGE analysis of 4 μg of protein separated using a 4-15% mini-PROTEAN TGX gel run at 120 V for 60 minutes. Total protein content was visualized via Coomassie Blue staining and band intensities were quantified using the gel analysis tool of FIJI.5 (B) Deconvoluted mass spectrum of MicD-F sample. (C) Deconvoluted mass spectrum of ArtGox sample. As expected, the N-terminal methionine residue was found to be excised from both enzymes during expression in E. coli.8,9

FIG. 15. Effect of time on the extent of MicD-F-promoted cyclodehydration of the model peptide ICAYDG. Examination of the extracted ion chromatograms at 1, 2, and 4 h indicated that reaction of ICAYDG with 5 mol % MicD-F was complete after 4 hours at pH 8.0 and 37° C.

FIG. 16. UHPLC analysis of MicD-F-catalyzed cyclodehydration of substrates 1a-i. Thiazolines 2a-i are the sole reaction products when substrates 1a-i are treated with 5 mol % MicD-F at pH 8.0 and 37° C. for 4 hours. Each panel shows the UHPLC trace (detection at 280 nm) of the indicated reaction mixture at the 4 hour time point for reactions that contained or lacked MicD-F. Contaminating signals identified from the absorbance baseline (dashed absorbance trace) are highlighted in grey and marked with an asterisk.

FIG. 17A-C. Optimization of conditions for one-pot cyclodehydration and dehydrogenation of the model peptide ICAYDG by MicD-F and ArtGox. (A) Reaction of the substrate ICAYDG with 5 mol % MicD-F and 0-80 mol % ArtGox at pH 8.0 and 37° C. Mass spectra of reaction mixtures containing the indicated concentration of ArtGox at (B) 4 hours and (C) 16 hours show that 40 mol % ArtGox and 16 hours of reaction are required to yield full conversion to the thiazole product. The ratio between the +1 amu isotope and the +2 amu isotope (boxed, red) was monitored to confirm complete conversion to the desired thiazole product.

FIG. 18. UHPLC analysis of one-pot cyclodehydration and dehydrogenation of substrates 1a-i by MicD-F and ArtGox. Thiazoles 3a-e are the sole reaction products when substrates 1a-e are treated with 5 mol % MicD-F and 40 mol % ArtGox at pH 8.0 and 37° C. for 16 hours. Excess flavin mononucleotide coelutes with substrates 1f-i. Therefore, UHPLC analysis cannot determine the extent of reaction for these substrates. Each panel shows the UHPLC trace (detection at 280 nm) of the indicated reaction mixture at the 16 hour time point for reactions that contained MicD-F and ArtGox (red absorbance trace). Retention time of the parent peptide as given by FIG. 16 is provided for reference (black absorbance trace). Contaminating signals identified from the absorbance baseline (dashed absorbance trace) are highlighted in grey and marked with an asterisk.

FIG. 19. Optimizing reaction conditions for the MicD-F-catalyzed cyclodehydration of the model peptide ICIAYDG. Extracted ion monitoring indicated that treatment of ICIAYDG with 50 mol % MicD-F for 24 hours at pH 8.0 and 37° C. led to complete reaction.

FIG. 20. Optimizing reaction conditions for one-pot cyclodehydration and dehydrogenation of the model peptide ICIAYDG in the presence of MicD-F and ArtGox. The ratio between the +1 amu isotope and the +2 amu isotope (boxed, red) was monitored to confirm complete conversion to the desired thiazole product. The mass spectral data indicates that reaction with 50 mol % MicD-F and 8 mol % ArtGox for 24 hours at pH 8.0 and 37° C. was sufficient for complete conversion to the thiazole-modified peptide.

FIG. 21A-C. Effect of pH on one-pot cyclodehydration and dehydrogenation of peptides containing non-α-amino acid monomers at the −1 site. (A) MicD-F and ArtGox reactions of substrates with non-α-amino acid monomers immediately C-terminal to the site of cyclization. (B) Yields of thiazoline 5(a-i) and thiazole (6(a-i)) products obtained for polypeptide substrates with non-α-amino acid monomers at position −1, immediately C-terminal to the reaction site. (C) Extracted ion chromatograms illustrating the products of MicD-F and ArtGox-catalyzed reactions.

FIG. 22A-C. Tolerance of non-α-amino acid backbones at the site of cyclization. (A) Cyclodehydration reaction of substrates ITAYDG and IXAYDG (where X=L- or D-β³ threonine) with 50 mol % MicD-F at pH 8.0 or pH 9.0 and 37° C. for 24 hours. These conditions led to significant cyclodehydration of the model substrate ITAYDG. However, no cyclodehydration of substrates containing a β³-amino acid was observed at either (B) pH 8.0 or (C) pH 9.0.

FIG. 23. Characterization of mCherry variants purified via Talon affinity chromatography. SDS-PAGE analysis of 4 g of protein separated using a 4-15% mini-PROTEAN TGX gel run at 200 V for 30 minutes. Total protein content was visualized via Coomassie Blue staining. As previously reported,10,11 boiling of the mCherry constructs for SDS-PAGE analysis yielded two lower molecular weight fragment bands due to backbone hydrolysis. Band intensities were quantified using Bio-Rad Image Lab Software (Hercules, CA).

FIG. 24A-E. Characterization of mCherry variants purified via Talon affinity chromatography. Deconvoluted mass spectrum of (A) mCherryC+, (B) mCherryC−, (C) mCherry174+, (D) mCherry192+, and (E) mCherry211+. Parent protein is shaded in grey and mature protein containing the mCherry chromophore (−22 Da) is shaded in red. The mCherry211+ variant, which displayed poor chromophore maturation, was excluded from further analysis due to presumed folding defects.

FIG. 25A-E. Cyclodehydration of (A) mCherry variants upon treatment with 50 mol % MicD-F (pH 9.0, 37° C.) for 24 hours. Under these conditions (B) mCherryC+ was nearly completely converted to the cyclodehydration product while (C) mCherryC−, (D) mCherry174+, and (E) mCherry192+ gave no evidence of cyclodehydration.

FIG. 26A-E. Cyclodehydration of (A) mCherry variants upon treatment with 50 mol % MicD-F (pH 9.0, 42° C.) for 24 hours. Under these conditions (B) mCherryC+ was nearly completely converted to the cyclodehydration product while (C) mCherryC−, (D) mCherry174+, and (E) mCherry192+ gave no evidence of cyclodehydration.

FIG. 27. Characterization of Rop variants purified via Talon affinity chromatography. All contained an N-terminal FLAG purification tag, and one of five ten-amino acid sequences in place of the native Asp30-Ala31 sequence of Rop. They also all contained a C-terminal 6×His purification tag. SDS-PAGE analysis of 4 g of protein separated using a 4-15% mini-PROTEAN TGX gel run at 200 V for 30 minutes. Total protein content was visualized via Coomassie Blue staining. Band intensities were quantified using Bio-Rad Image Lab Software (Hercules, CA).

FIG. 28A-E. Characterization of Rop variants purified via Talon affinity chromatography. Deconvoluted mass spectrum of (A) RopN, (B) RopM, (C) RopC, (D) RopCG4, and (E) RopC−.

FIG. 29A-B. (A) Size-exclusion chromatography (SEC) traces for purified samples of RopC, RopM, RopN, RopC-U, and RopC-Z both before and after refolding. Note that RopC-Z remains heterogeneous even after temperature-induced refolding. (B) Temperature dependent CD analysis of RopN, RopM, and RopC.

FIG. 30A-B. Optimizing conditions for the cyclodehydration of RopC by MicD-F. (A) Effect of [MicD-F] on the conversion of RopC (grey bars) into RopC-U (green bars) after 16 h at 25° C. Conversion was minimal even at the highest [MicD-F] used (50 μM). (B) Effect of [MicD-F] on the conversion of RopC (grey bars) into RopC-U (green bars) after 16 h at 37° C. Conversion was highest at the highest [MicD-F] used (50 μM).

FIG. 31. Optimization of one-pot cyclodehydration and dehydrogenation of RopC by MicD-F and ArtGox. The deconvoluted mass spectrum indicated that reaction with 50 mol % MicD-F and 80 mol % ArtGox for 24 hours at pH 9.0 and 37° C. was sufficient for complete conversion to the thiazole-modified protein.

FIG. 32A-D. Cyclodehydration of RopC is Cys44-specific and requires the C-terminal AYD recognition sequence. A) Control Rop constructs, RopC- and RopCG4, were designed to test the importance of Cys44 and the C-terminal AYD recognition sequence in cyclodehydration of RopC by MicD-F. B) RopC demonstrated near complete cyclodehydration upon treatment with 50 mol % MicD-F for 16 hours at 37° C. and pH 9.0. C) RopC−, which is a cysteine to alanine mutant of RopC, displayed no cyclodehydration under the tested reaction conditions. D) RopCG4, which substitutes the C-terminal AYD recognition sequence with a triglycine sequence, also displayed no cyclodehydration under the tested reaction conditions.

FIG. 33. RopC-Z requires refolding to display cooperative unfolding behavior. An initial temperature melt monitoring ellipticity at 222 nm yielded a monotonically increasing melt curve (squares). After returning the sample to 25° C. a second temperature melt monitoring ellipticity at 222 nm yielded a two-state transition (circles) characteristic of cooperative unfolding. Data reported (n=1) is representative of behavior observed in three trials, two of which monitored the initial melt at 208 nm instead of 222 nm.

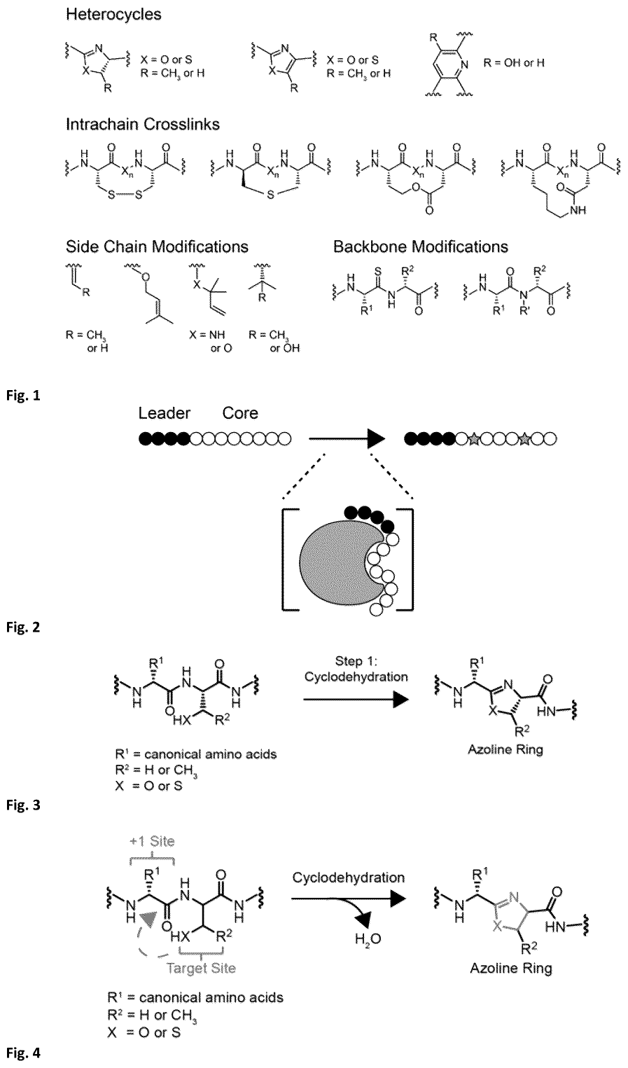

### DESCRIPTION OF PARTICULAR EMBODIMENTS OF THE INVENTION

Unless contraindicated or noted otherwise, in these descriptions and throughout this specification, the terms “a” and “an” mean one or more, the term “or” means and/or. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

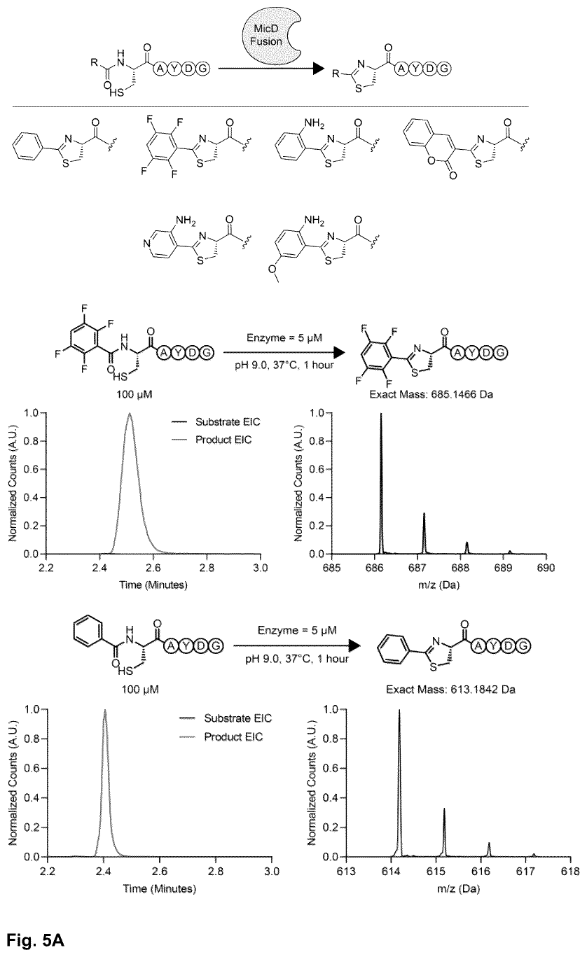

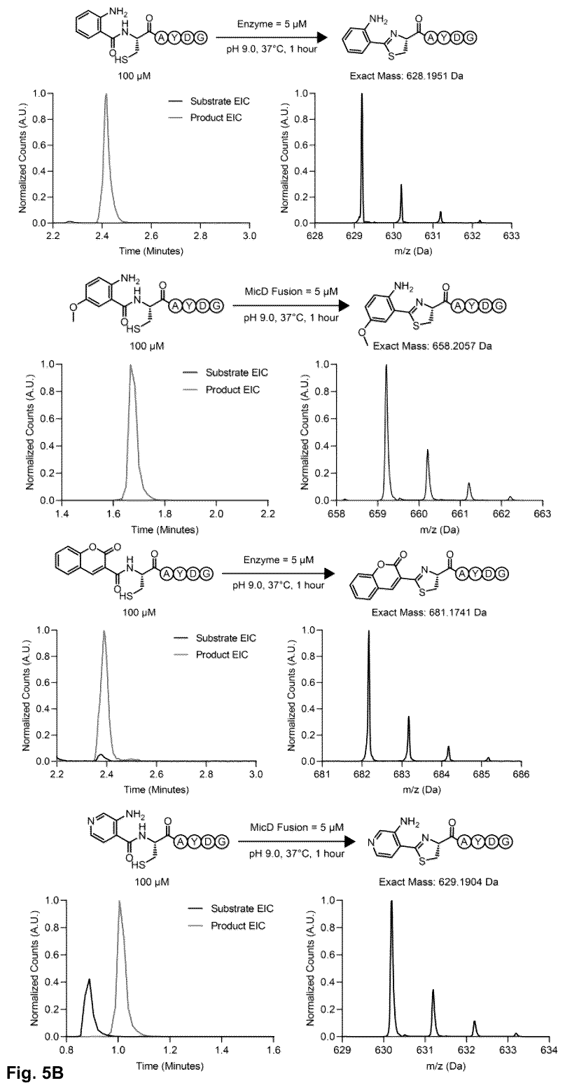

Expanded substrate scope at +1 position. Tolerance to diverse aromatic +1 site residues. We explored the extent to which MicD fusion would accept non-α-amino acid substrates at the +1 position (FIG. 4). We focused first on diverse benzoic acid derivatives, and discovered that peptides containing diverse aromatic groups at the +1 position were excellent substrates for MicD fusion, undergoing cyclization to the products shown in virtually quantitative yield under standard reaction conditions (FIG. 5A-B).

Tolerance to diverse aromatic +1 site residues. We explored the extent to which MicD fusion would accept non-α-amino acid substrates at the +1 position (FIG. 4). We focused first on diverse benzoic acid derivatives, and discovered that peptides containing diverse aromatic groups at the +1 position were excellent substrates for MicD fusion, undergoing cyclization to the products shown in virtually quantitative yield under standard reaction conditions (FIG. 5A-B).

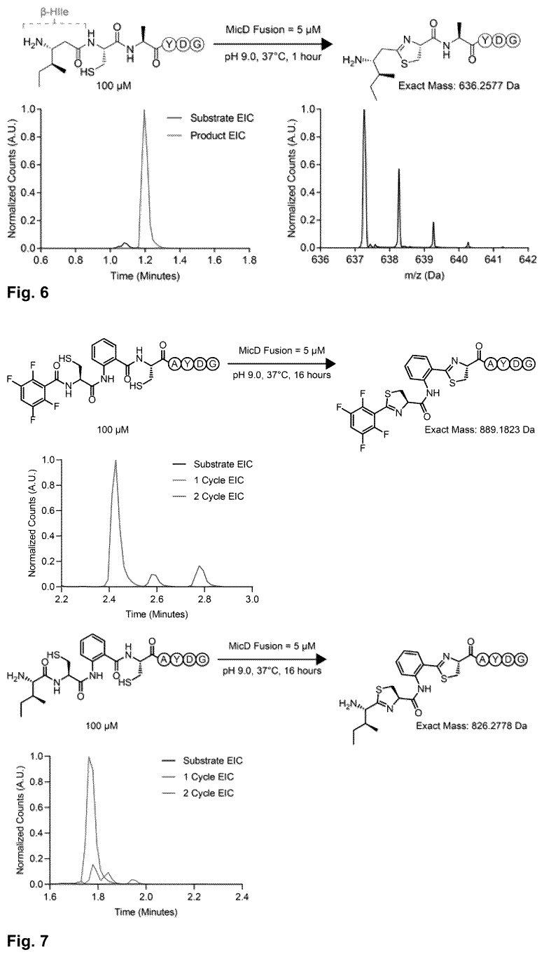

Tolerance to beta-β-amino acids at +1 site. We explored the extent to which MicD fusion would accept b3-amino acid substrates at the +1 position (FIG. 6). We demonstrated that b3-amino acids are excellent substrates for MicD fusion, undergoing cyclization to the products shown in virtually quantitative yield under standard reaction conditions (FIG. 6).

Tolerance to diverse −1 site residues. We next sought to explore the extent to which MicD fusion would accept non-α-amino acid substrates at the −1 position (FIG. 7). We demonstrated that peptides containing aramid monomers at the −1 position were also excellent substrates for MicD fusion, undergoing cyclization to the products shown in virtually quantitative yield under standard reaction conditions (FIG. 7).

The invention provides numerous practical applications. Currently, if one sought a protein therapeutic fused to a RiPP natural product, that material would need to be synthesized in multiple steps and would likely include a chemical bio-conjugation step that often requires significant optimization. Here, the desired material can be prepared in a single step, even in situ, and purified only once. The invention can be used to improve the ease of production of therapeutic proteins, (b) improve the thermal and proteolytic stability of therapeutic proteins, (c) add a targeting function to a therapeutic protein, (d) modulate the immune response to a therapeutic protein, etc.

### Example: Redirecting RiPP Biosynthetic Enzymes to Proteins and Backbone-Modified Substrates

Abstract: Ribosomally synthesized and post-translationally modified peptides (RiPPs) are peptide-derived natural products that include the FDA-approved analgesic ziconotide1,2 as well as compounds with potent antibiotic, antiviral, and anticancer properties.3 RiPP enzymes known as cyclodehydratases and dehydrogenases represent an exceptionally well-studied enzyme class.3 These enzymes work together to catalyze intramolecular, inter-residue condensation3,4 and aromatization reactions that install oxazoline/oxazole and thiazoline/thiazole heterocycles within ribosomally produced polypeptide chains. Here we show that the previously reported enzymes MicD-F and ArtGox accept backbone-modified monomers-including aramids and beta-amino acids-within leader-free polypeptides, even at positions immediately preceding or following the site of cyclization/dehydrogenation. The products are sequence-defined chemical polymers with multiple, diverse, non-α-amino acid subunits. We show further that MicD-F and ArtGox can install heterocyclic backbones within protein loops and linkers without disrupting the native tertiary fold. Calculations reveal the extent to which these heterocycles restrict conformational space; they also eliminate a peptide bond-both features could improve the stability or add function to linker sequences now commonplace in emerging biotherapeutics. Moreover, as thiazoles and thiazoline heterocycles are replete in natural products,5-7 small molecule drugs,8,9 and peptide-mimetic therapeutics,10 their installation in protein-based biotherapeutics can be deployed to improve or augment performance, activity, stability, and/or selectivity. This invention provides a general strategy to expand the chemical diversity of the proteome beyond and in synergy with what can now be accomplished by expanding the genetic code.

### INTRODUCTION

Ribosomally synthesized and post-translationally modified peptides (RiPPs) are peptide-derived natural products that include the FDA-approved analgesic ziconotide1,2 as well as compounds with potent antibiotic, antiviral, and anticancer properties.3 RIPP biosynthesis begins with a ribosomally synthesized polypeptide whose N-terminal leader sequence (−20-110 aa) recruits one or more endogenous enzymes capable of diverse post-translational modification (PTM) of an adjacent C-terminal substrate sequence.3,11 Researchers have leveraged this leader-dependent mechanism to direct RiPP PTM enzymes to C-terminal substrate sequences containing diverse non-canonical α-amino acids (nc-α-AAs).12,13

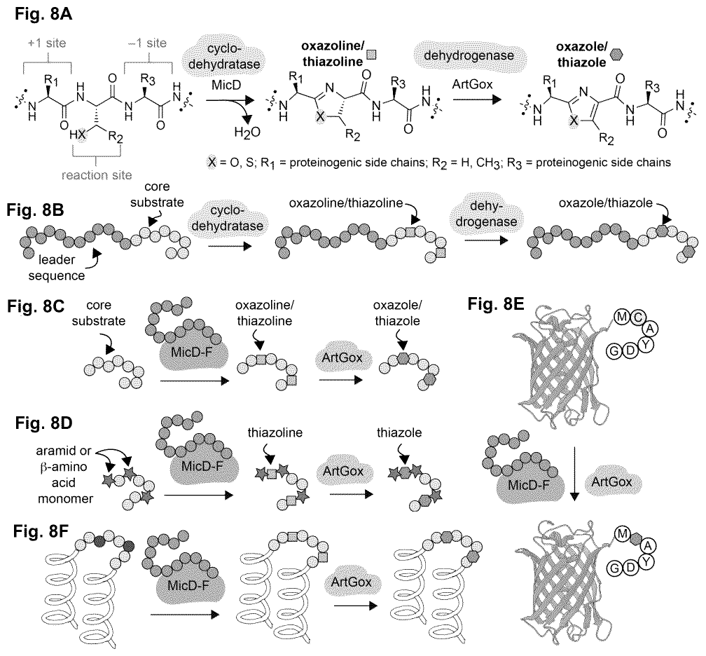

Cyclodehydratases and dehydrogenases represent an exceptionally well-studied class of RiPP enzymes.3 These enzymes work together to catalyze intramolecular cyclization3,4 and subsequent aromatization reactions that install oxazoline/oxazole and thiazoline/thiazole heterocycles within polypeptide chains (FIG. 8A, B). Previous work has shown that the cyclodehydratases PatD14 and TruD15 support leader sequence-dependent oxazoline/thiazoline formation within substrates containing nc-α-AAs adjacent to 16 or at the cyclization site itself.17-19 In related work, it was shown that a chimeric leader peptide could direct the cyclodehydratase LynD15 and the dehydrogenase TbtE20 to install thiazol(in)es within substrates containing nc-α-AAs adjacent to the cyclization site.21 Finally, reconstituted lactazole biosynthesis,22 including the cyclodehydratase-dehydrogenase pair LazDE/LazF, was found to install oxazoles and thiazoles within polypeptide substrates containing α-hydroxy, N-methyl, cyclic α-, and β³-amino acids23 at sites distal from the site of heterocyclization (>4 residues away).

Previous work has also shown that certain cyclodehydratase enzymes retain activity for leader sequence-free substrates when the leader peptide is provided in trans (FIG. 8C).24,²⁵ Building on this observation, Naismith and coworkers engineered a family of cyclodehydratases in which the leader peptide is fused to the N-terminus of the cyclodehydratase catalyst as opposed to the N-terminus of the substrate polypeptide. These constitutively activated enzymes, notably LynD Fusion (LynD-F)²⁵ and MicD Fusion (MicD-F)26 act in a leader peptide-independent manner to promote the cyclodehydration of polypeptides containing a C-terminal Ala-Tyr-Asp (AYD) recognition sequence.4, 25-28 In complementary work, Schmidt and coworkers demonstrated that two dehydrogenases, ArtGox and ThcOx, also accept leaderless peptide substrates.29 Taken together, these enzymes represent a fully leader-free route towards polypeptides (and proteins, vide infra) containing mRNA-programmed thiazole and oxazole linkages. Indeed, some tolerance for non-canonical α-amino acid residues has been reported: LynD-F was shown to install a thiazoline in a peptide substrate containing 3-azido-L-alanine positioned 4 residues away from the site of cyclization,28 and the combination of LynD-F and ArtGox installed a thiazole in an AYD-containing peptide with a polyethylene glycol spacer 2 residues from the site of cyclization.27

Here we report that MicD-F26,29 and ArtGox26,29 act together to process polypeptide substrates containing diverse translation-compatible30-35 aramid and β-amino acid monomers, even at sites directly flanking the reaction site (FIG. 8D). We show further that MicD-F26,29 and ArtGox26,29 process substrates even when the CAYD sequence is positioned at the C-terminus of mCherry, a large β-barrel protein, or embedded within the loop of the dimeric α-helical bundle protein Rop; the products are folded, globular proteins containing a conformationally restricted, fully unnatural, heterocyclic backbone. To the best of our knowledge, these studies represent the first example of leader-free azol(in)e biosynthesis within polypeptides containing diverse non-α-amino acid monomers flanking the site of cyclization, and the first report of a cooperatively folded protein containing a post-translationally installed heterocyclic ring.36 The effects of the embedded heterocycle on local conformational flexibility are examined computationally, providing important insight into the backbone restrictions that could be leveraged to improve the physio-chemical properties of therapeutic proteins. This work represents a general strategy to expand the chemical diversity of the proteome beyond and in synergy with what can now be accomplished by expanding the genetic code.

### Results

MicD-F and ArtGox Accept Substrates with Diverse Structures at the +1 Site

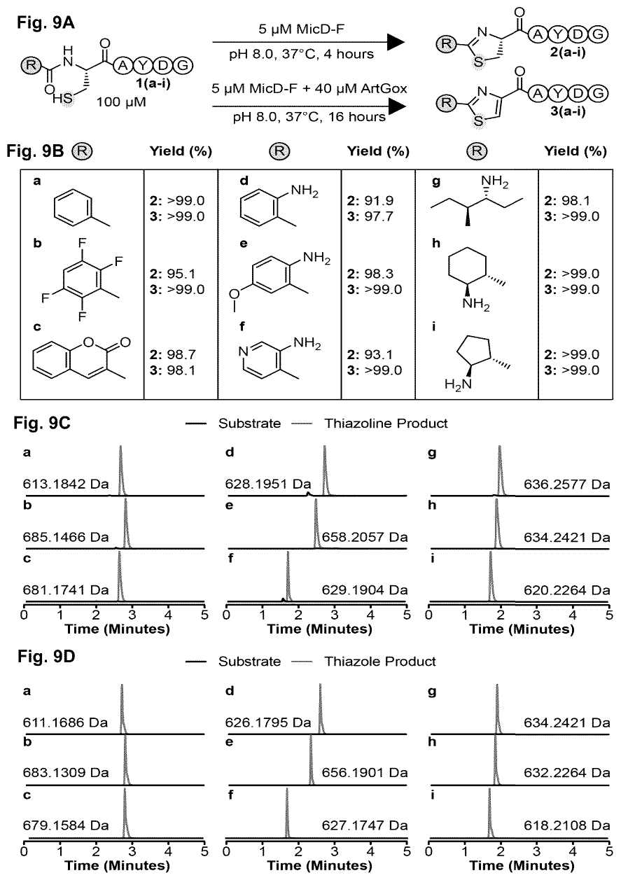

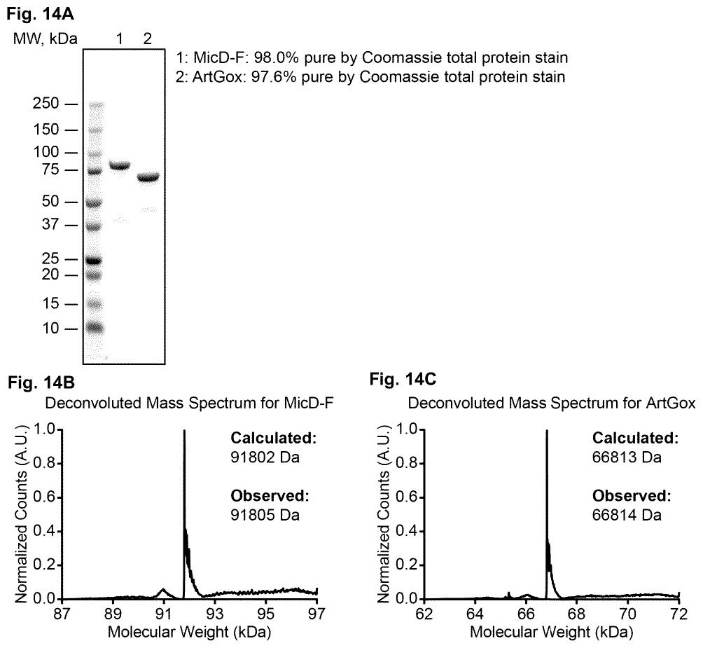

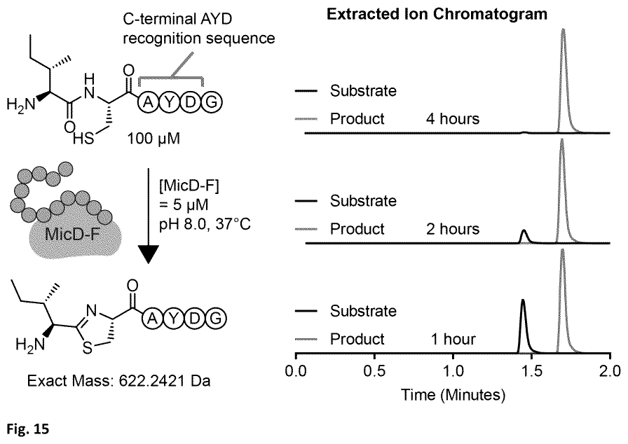

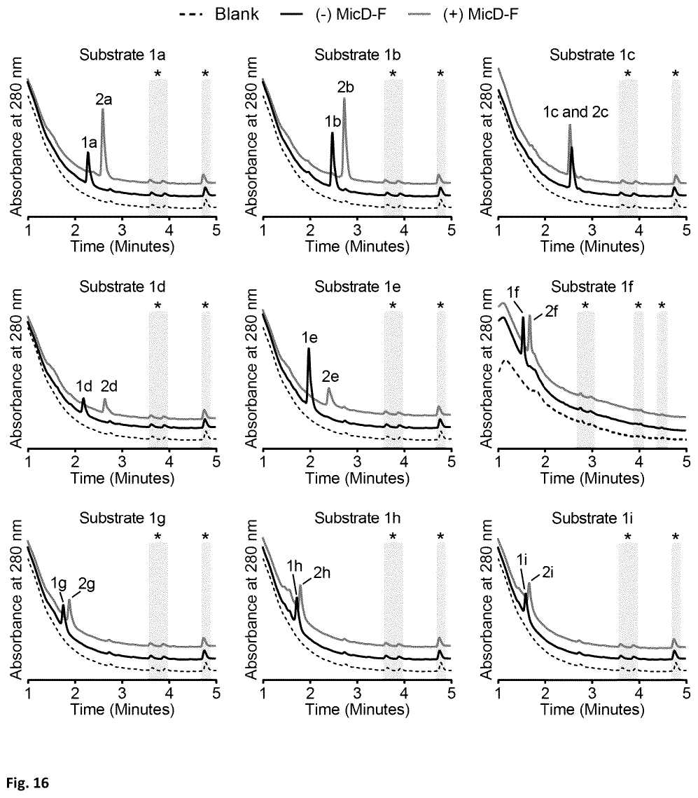

We began by exploring the tolerance of MicD-F for sequences containing non-α-amino acid monomers at the +1 site (FIG. 8A and FIG. 9A-B). A series of nine potential substrates were prepared in which a non-α-amino acid preceded the reaction site (substrates 1(a-i)). Monomers evaluated included arenes, aramids, fluorophores, and linear and cyclic β-amino acids. All substrates contained a C-terminal AYD recognition sequence39 and were incubated with MicD-F (FIG. 14A-B) (5 mol %) under mild conditions (pH 8.0, 37° C., 4 h) that resulted in complete conversion of ICAYDG, a substrate with the natural α-amino acid Ile at the +1 site (FIG. 15). Cyclization was analyzed initially via liquid chromatography-mass spectrometry (LC-MS) and the extent of product formation estimated by integrating the extracted ion chromatogram (FIG. 9B-C). Virtually every substrate examined underwent MicD-F-catalyzed cyclization to the corresponding thiazoline under these conditions. Substrates containing electron-withdrawing or donating aromatic rings, bulky multi-ring systems, and linear and cyclic β-amino acids were all cyclized efficiently by MicD-F, with yields between 91 and 99% (products 2(a-i)). UHPLC analysis of each reaction mixture confirmed that thiazolines 2a-i were the sole reaction products under these conditions (FIG. 16). It is notable that monomers with highly divergent structures are accepted almost equally by MicD-F, suggesting that the +1 residue interacts minimally if at all with the enzyme active site.

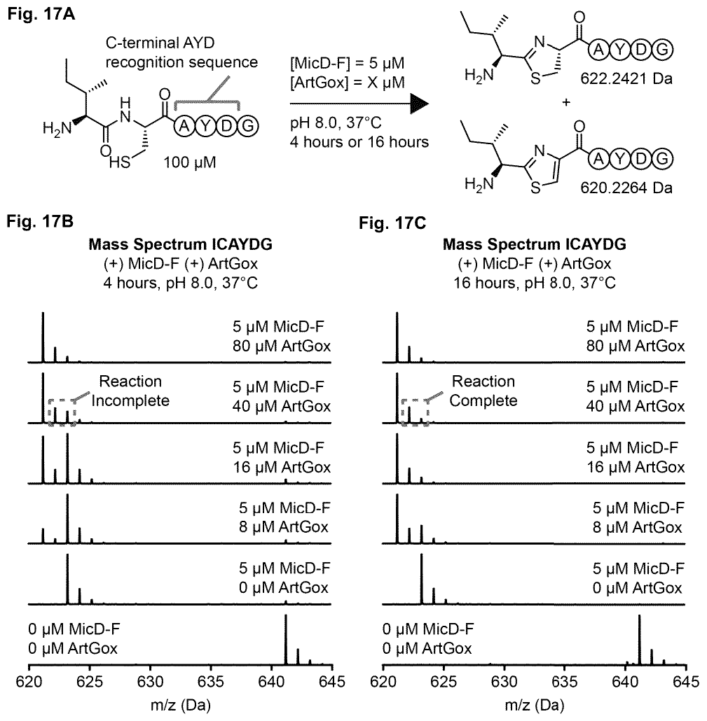

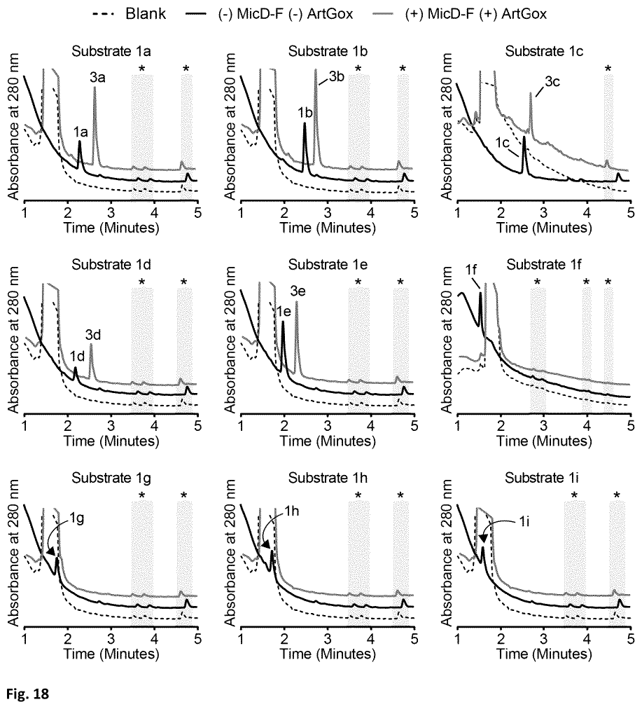

Next, we explored whether MicD-F and ArtGox could act in synergy to convert peptides containing non-α-amino acids at the +1 site directly into the corresponding thiazoles 3(a-i) (FIG. 9A). Substrates 1(a-i) were incubated with MicD-F (5 mol %) and ArtGox (FIGS. 14A and C) (40 mol %) under conditions (pH 8.0, 37° C., 16 h) that resulted in complete two-step conversion of ICAYDG into the corresponding thiazole product (FIG. 17A-C). ArtGox efficiently oxidized each thiazoline to the corresponding thiazole in yields that exceeded 97% over the two steps for every example (products 3(a-i)) (FIGS. 9B and D). UHPLC analysis of each reaction mixture confirmed that thiazoles 3a-e were the sole reaction product. Coelution with excess flavin mononucleotide precluded UHPLC analysis of thiazoles 3f-i (FIG. 18). These results indicate that MicD-F and ArtGox tolerate diverse non-proteinogenic, non-α-amino acid monomers at the +1 site. Many of these non-α-amino acid monomers have been installed at the N-termini of ribosomally translated peptides in vitro,^{33,34,40-41} suggesting a path towards proteins and polypeptides with highly unique N-terminal appendages.

MicD-F and ArtGox Accept Substrates with Diverse Structures at the −1 Site

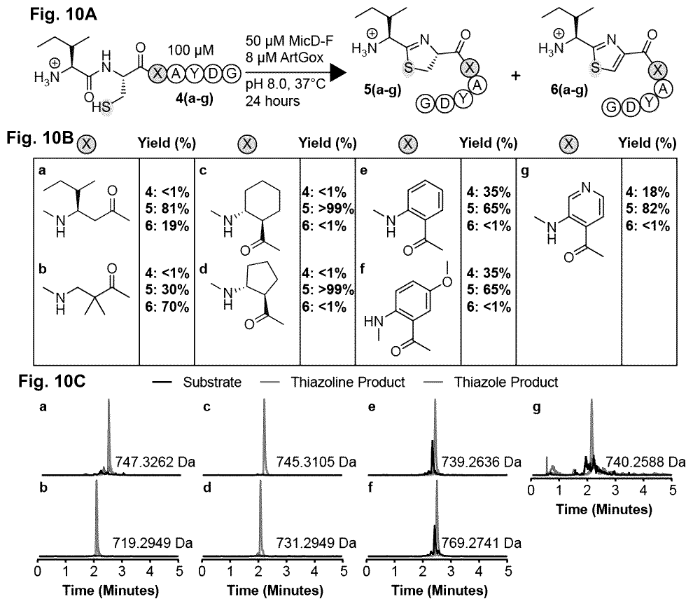

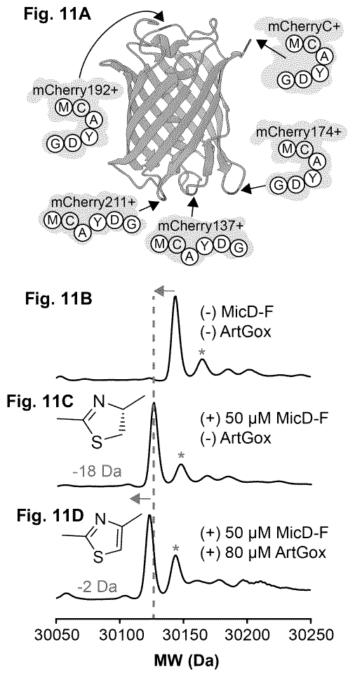

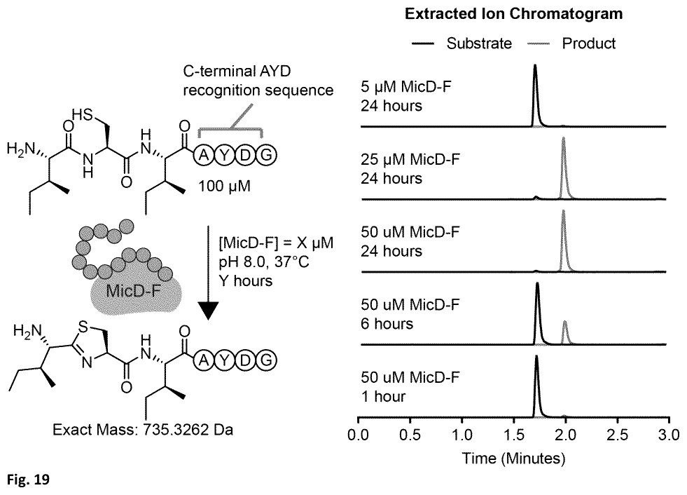

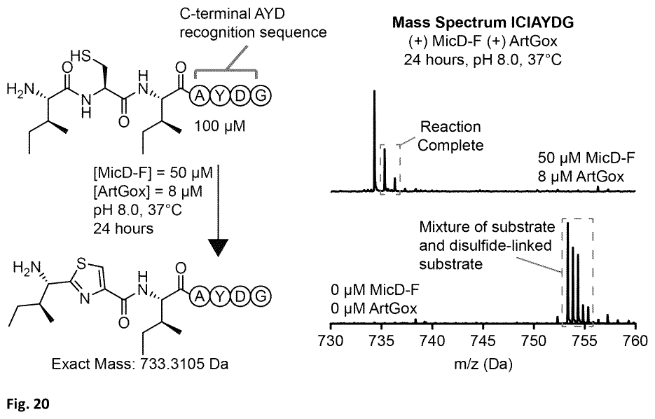

Next we explored whether MicD-F and ArtGox would accept leader-free polypeptide substrates containing non-α-amino acid monomers at the −1 site (FIG. 10A-C). We explored a diverse array of monomers—β³-amino acids, β²-amino acids, cyclic β²,β³-amino acids, as well as substituted and unsubstituted aramids. Notably, it was found that inserting an α-amino acid (Ile) residue between the site of cyclization and the C-terminal AYD motif necessitated higher concentrations (50 mol %) of MicD-F and up to 24 h reaction time to complete the cyclodehydration reaction (FIG. 19).

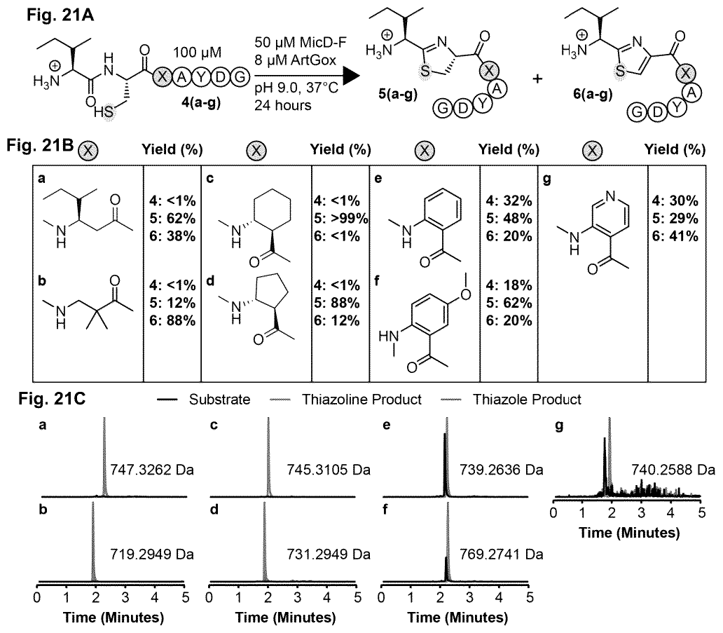

All −1 site substrates (substrates 4(a-g), FIG. 10A-B) contained a C-terminal AYD recognition sequence39 and were incubated with MicD-F (50 mol %) and ArtGox (8 mol %) under conditions (pH 8.0, 37° C., 24 h) that resulted in complete conversion of a substrate with a natural α-amino acid at the −1 site to the corresponding thiazole (FIG. 20). Reactions were analyzed as described above. Under these conditions, every peptide evaluated was a substrate for MicD-F and a few were substrates for both MicD-F and ArtGox (FIG. 10B-C). Substrates containing β³-amino acid-, β²-amino acid-, or cyclic β²,β³-amino acids at the −1 site (substrates 4(a-d)), were fully consumed under these conditions (<1% unmodified peptide). Those with β³-alkyl substituents (4a, c, and d) were converted cleanly into the corresponding thiazolines 5a, c, and d, with little (4a) or no (4c,d) thiazole formation. In contrast, substrate 4b, with geminal β²-methyl substituents, was converted into a 30/70 mixture of thiazoline 5b and thiazole 6b. Substrates 4e-g containing aramid monomers at the −1 position reacted more slowly under these conditions, producing the analogous thiazoline products in 65-85% yield after 24 h reaction at pH 8 (FIG. 10B-C). Surprisingly, while all substrates containing +1 site modifications were efficiently oxidized to the corresponding thiazole (FIGS. 9B and D), only the substrate containing a β²-amino acid modification at the −1 site was efficiently oxidized by ArtGox (70%) (FIG. 10B-C). With the exception of substrate 4c, increasing the pH to 9.0 promoted formation of the desired thiazole product (FIG. 21B-C). However, even under these conditions only substrate 4b (88%) yielded greater than 41% thiazole product (FIG. 21B-C). These data indicate that MicD-F and ArtGox are both less tolerant of non-α-amino acid monomers at the −1 site than at the +1 site. ArtGox appears especially intolerant of substitution or sp² hybridization at the β³-position of substrates at the −1 site.

### MicD-F is Sensitive to Amino Acid Identity at the Cyclization Site

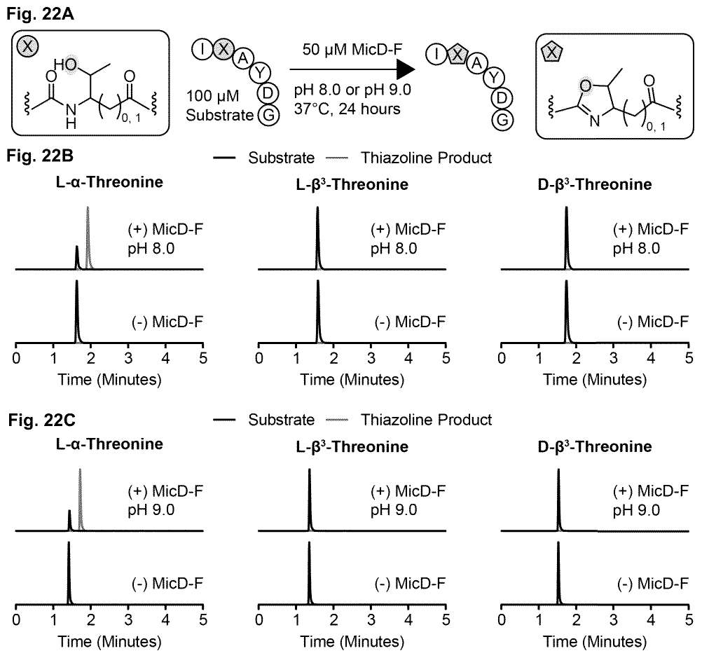

To complete the exploration of the substrate tolerance of MicD-F and ArtGox, we synthesized a set of potential substrates containing a non-α-amino acid directly at the cyclization site. Each contained a C-terminal AYD sequence preceded by either L-β³- or D-β³-threonine (FIG. 22A). Incubation of these substrates with MicD-F (50 mol %) under conditions (pH 8.0, 37° C., 24 h) that resulted in substantial cyclization of a substrate containing L-α-threonine at the cyclization site led to no detectable cyclization (<1%) (FIG. 22B). Even at pH 9.0 no cyclization occurred (FIG. 22C), indicating that MicD-F is highly sensitive to amino acid identity at the site of cyclization. This result is in line with previous work that demonstrated the cyclodehydratase PatD failed to react with substrates containing D-α-threonine at the cyclization site.18

### Redirecting RiPP Biosynthetic Enzymes to Intact Folded Proteins

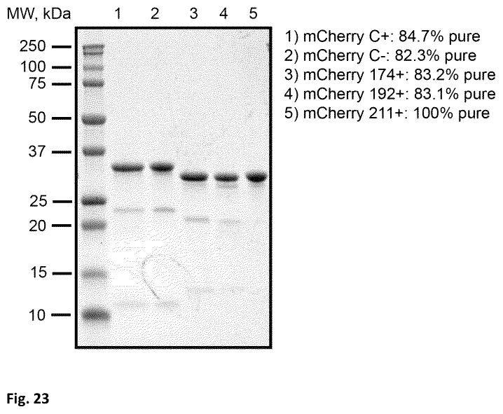

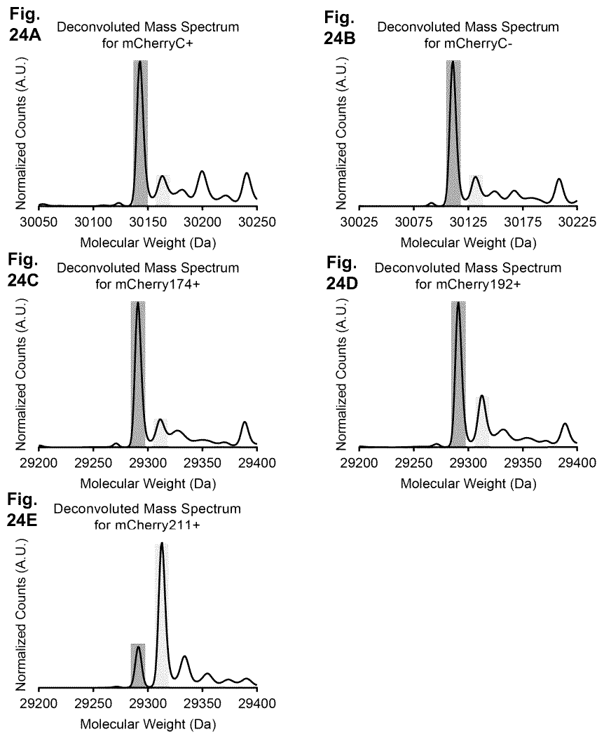

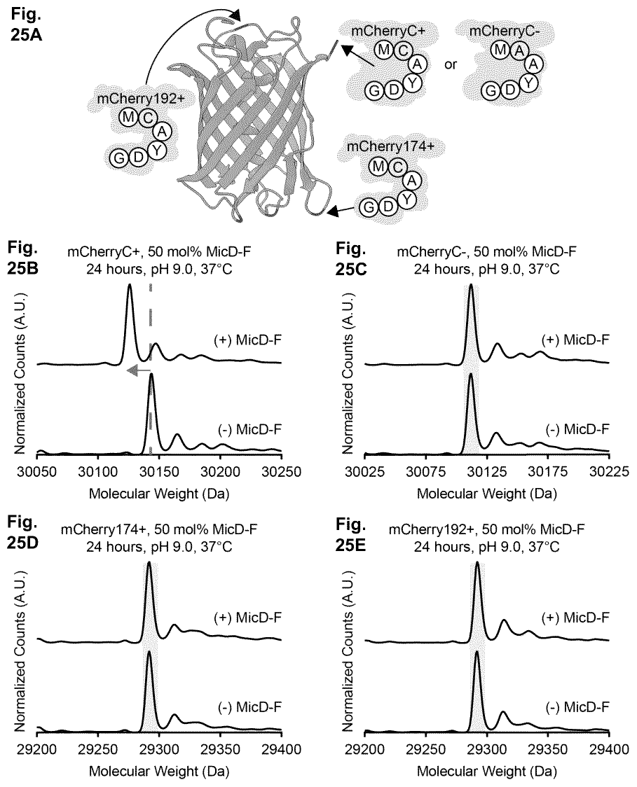

Thiazolines and thiazole are replete in natural products42-44 and synthetic drug-like small molecules,^{8,9} and calculations confirm the expected decrease in conformational freedom that derives from aromatic and/or sp² character within the peptide backbone.45 This finding and the leader-independent nature of MicD-F and ArtGox-mediated thiazol(in)e biosynthesis inspired us to explore substrates in which the site of cyclodehydration/dehydrogenation is embedded within a stable protein fold (FIG. 11A-D). We first asked whether MicD-F and ArtGox could install thiazol(in)e linkages within loops and/or at the termini of mCherry. mCherry is a prototypic fluorescent beta-barrel protein derived from DsRed, isolated originally from Discosoma sea anemones.46 We cloned, expressed, and purified a set of mCherry variants in which the core sequence MCAYDG was appended to the mCherry C-terminus (mCherryC+) or inserted into a loop immediately downstream of residues D137 (mCherry137+), D174 (mCherry174+), V192 (mCherry192+), or E211 (mCherry211+) (FIG. 11A, Table 2, FIG. 23). Although mCherry137+ and mCherry211+ were partially/completely non-fluorescent or could not be purified, mCherryC+, mCherry174+ and mCherry192+ were soluble and fluorescent. In all three of these cases, mass spectrometry of the purified proteins showed the characteristic loss of 22 Da indicating chromophore maturation (FIG. 24A-E).

Treatment of mCherryC+ with 50 mol % MicD-F (pH 9.0, 24 hours, 37° C.) led to virtually complete conversion to the thiazoline product as indicated by a loss of water in the deconvoluted mass spectrum (FIG. 11B-C). No such mass change was observed in an analogous reaction containing mCherryC−, which carries the sequence MAAYDG in place of MCAYDG at the C-terminus, providing evidence that the observed cyclodehydration demanded a Cys residue immediately upstream of the AYD recognition sequence (FIG. 25B-C). Neither mCherry174+ nor mCherry192+ displayed the loss of water characteristic of successful cyclodehydration even after 24 hours at 37° C. (FIG. 25D-E). Nevertheless, we explored the potential for MicD-F and ArtGox to act in tandem to install an aromatic thiazole backbone in mCherryC+. Simultaneous treatment of mCherryC+ for 24 hours (pH 9.0, 37° C.) with MicD-F (50 mol %) and ArtGox (80 mol %) resulted in the expected −2 Da shift in the deconvoluted mass spectrum (FIG. 11D) relative to that of mCherryC+ treated with only MicD-F (FIG. 11C). This result indicates that the MicD-F/ArGox enzyme pair can post-translationally install an aromatic thiazole backbone within a structurally unconstrained region of a well-folded beta-barrel protein.

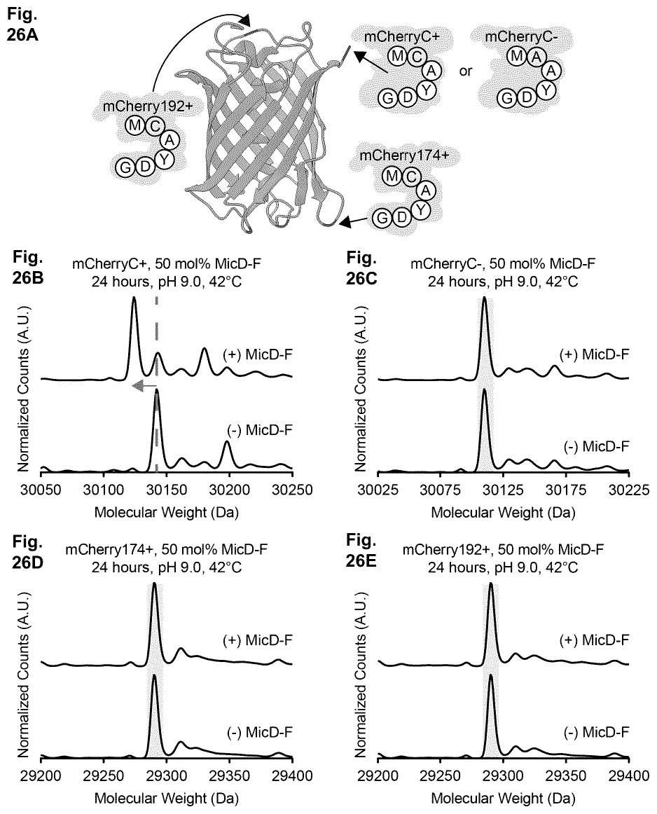

We hypothesized that the absence of cyclodehydration reactivity for mCherry174+ and mCherry192+ at 37° C. was due to neighboring structural elements that disfavor productive interaction with MicD-F and/or enzyme-promoted thiazoline formation. Therefore, we carried out a second set of cyclodehydration reactions at 42° C., the highest temperature at which MicD-F remained stable in our hands, which should increase the conformational flexibility of loop insertions. At this elevated temperature, mCherryC+ again displayed cysteine-specific loss of water characteristic of successful cyclodehydration (FIG. 26B-C). However, again neither mCherry174+ or mCherry192+ displayed the loss of water characteristic of successful cyclodehydration after 24 hours at 42° C. (FIG. 26D-E). It has been reported47 that the apparent melting temperature of mCherry is upwards of 90° C. Taken together with our results, this finding suggests that there is an inherent mismatch between the temperature stability of MicD-F and the thermodynamic stabilities of the mCherry loop insertions evaluated here.

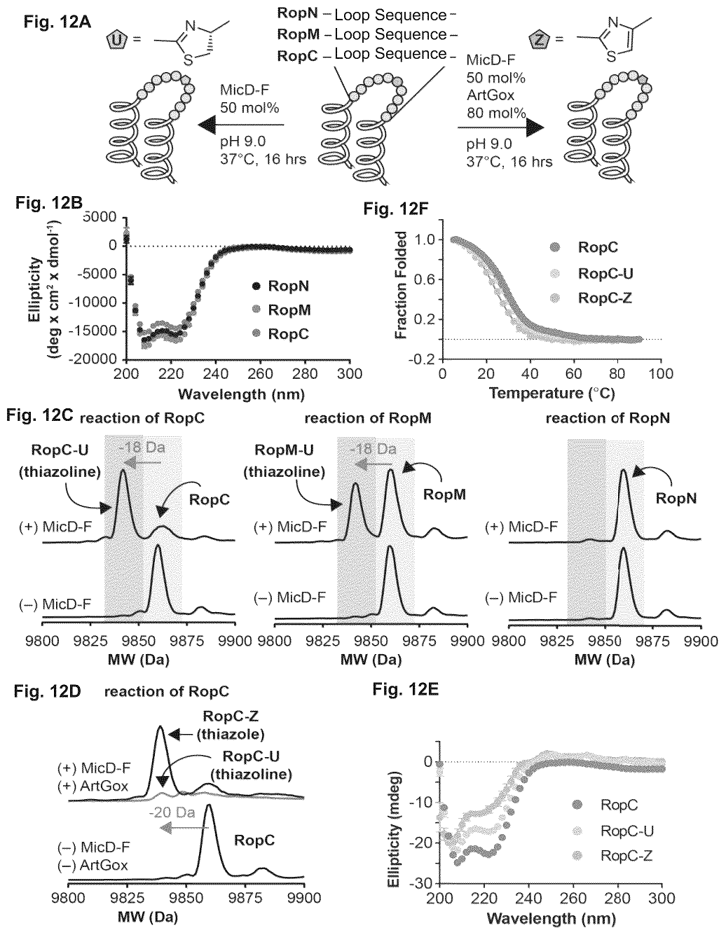

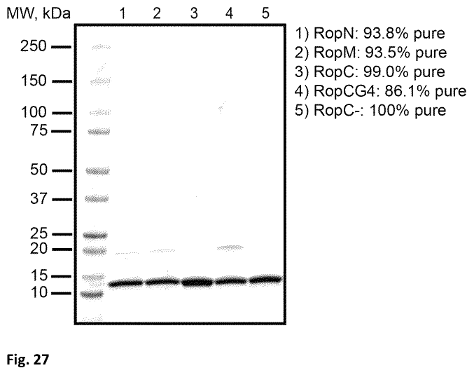

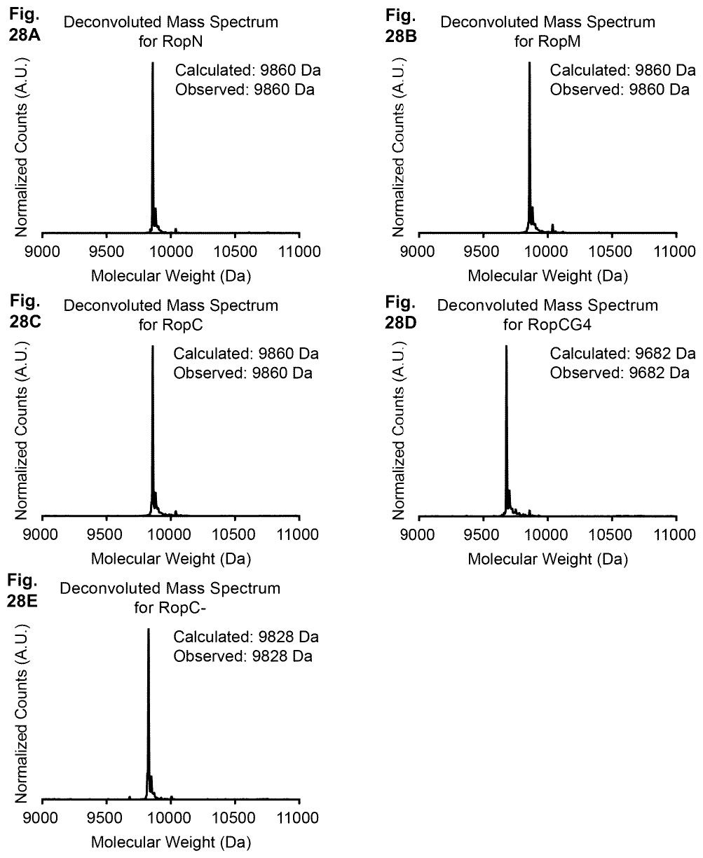

To test this hypothesis, we sought a folded, globular protein with a lower melting temperature than mCherry with the expectation that it would be more amenable to insertion of an internal thiazol(in)e linkage. Rop is a homodimeric four-helix bundle protein formed by the antiparallel association of two helix-turn-helix monomers.48 Regan and coworkers reported many years ago that the native two-residue turn in Rop could be replaced by up to ten glycine residues without loss of the native dimer structure. The Rop variant with the longest insertion-Gly₁₀-melted cooperatively at 50° C.,⁴⁹ suggesting that it might tolerate an internal, intra-loop thiazole or thiazoline (FIG. 12A). To test this hypothesis, we expressed and purified three Rop variants containing a single CAYD sequence embedded near the N-terminus (RopN), the C-terminus (RopC), or centrally (RopM) within a ten-residue glycine-rich loop (FIG. 12A, Table 3, FIG. 27, and FIG. 28A-E).

RopN Sequence
SEQ ID NO: 1
MDYKDDDDKMTKQEKTALNMARFIRSQTLTLLEKLNELMCAYDGGGGGD
EQADICESLHDHADELYRSCLARFGDDGENLHHHHHH
RopM Sequence
SEQ ID NO: 2
MDYKDDDDKMTKQEKTALNMARFIRSQTLTLLEKLNELGGMCAYDGGGD
EQADICESLHDHADELYRSCLARFGDDGENLHHHHHH
RopC Sequence
SEQ ID NO: 3
MDYKDDDDKMTKQEKTALNMARFIRSQTLTLLEKLNELGGGGMCAYDGD
EQADICESLHDHADELYRSCLARFGDDGENLHHHHHH
RopCG4 Sequence
SEQ ID NO: 4
MDYKDDDDKMTKQEKTALNMARFIRSQTLTLLEKLNELGGGGMCGGGGD
EQADICESLHDHADELYRSCLARFGDDGENLHHHHHH
RopC-Sequence
SEQ ID NO: 5
MDYKDDDDKMTKQEKTALNMARFIRSQTLTLLEKLNELGGGGMAAYDGD
EQADICESLHDHA
SEQ ID NO: 6
DELYRSCLARFGDDGENLHHHHHH

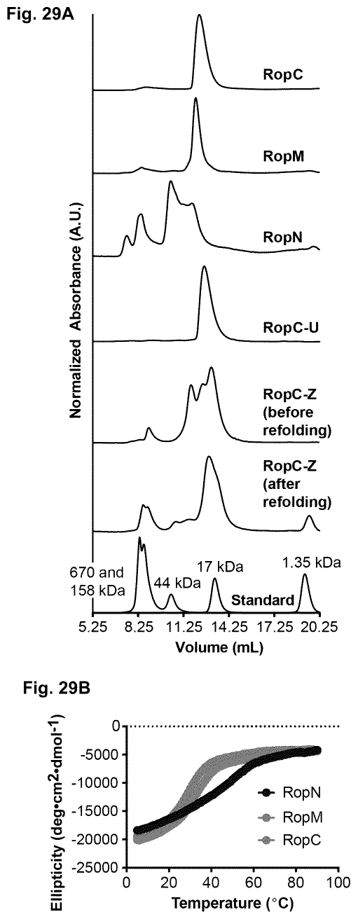

All three Rop variants exhibited high α-helical content at 20 μM as judged by wavelength-dependent CD measurements (FIG. 12B). RopC and RopM migrated as discrete dimers at 50 μM as judged by size-exclusion chromatography (SEC) and melted cooperatively and reversibly with T_{M} values of 28° C. and 32° C. (FIG. 29A-B). RopN, by contrast, migrated as a heterogeneous mixture upon SEC and melted non-cooperatively, albeit at a slightly higher apparent T_{M} (43° C.) perhaps because of disulfide formation (FIG. 29A-B).50

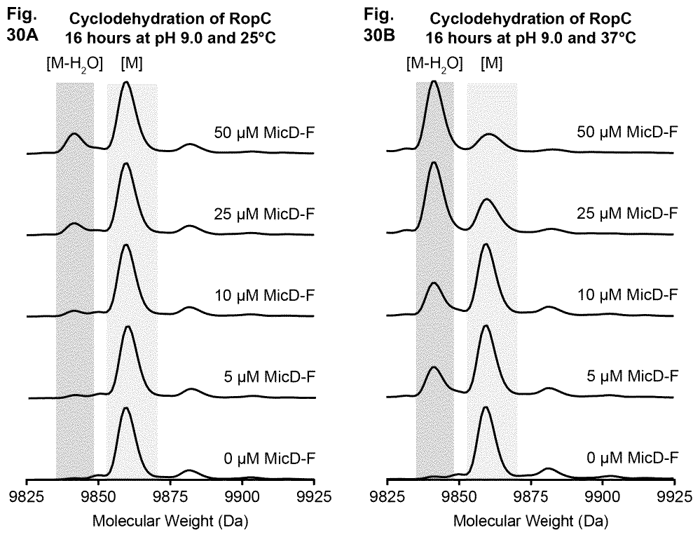

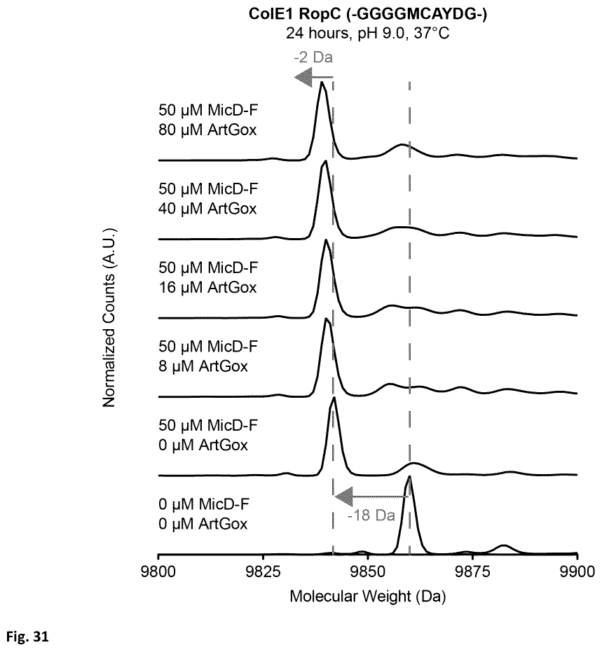

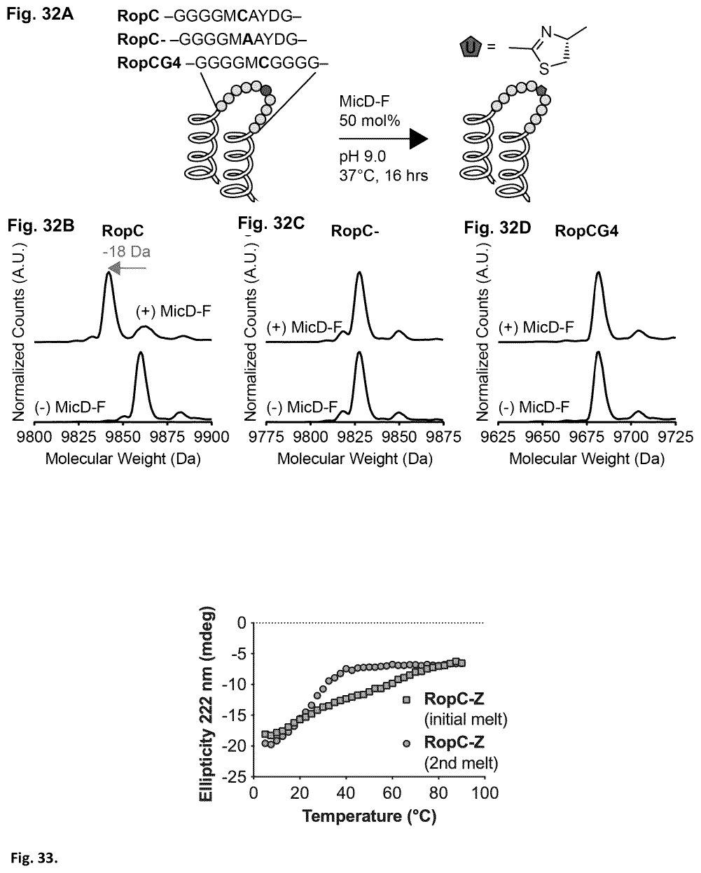

Although RopC, RopN, and RopM all contained the same CAYD recognition sequence, only one-RopC-underwent clean conversion into the corresponding thiazoline upon treatment with 50 mol % MicD-F (pH 9.0, 37° C., 16h). RopM reacted partially under these conditions and RopN was unreactive (FIG. 12C). Reaction of RopC to generate thiazoline RopC-U proceeded more slowly at 25° C. (FIG. 30A-B). RopC could be converted directly into the thiazole RopC-Z upon treatment with 50 mol % MicD-F and 80 mol % ArtGox (FIG. 12D, FIG. 31). No reaction was observed when the Cys residue within the RopC reaction site was replaced with Ala or when the C-terminal AYD sequence was replaced by GGG (FIG. 32A-D).

The products of the reaction of RopC with MicD-F (RopC-U) and with MicD-F and ArtGox (RopC-Z) were purified and analyzed by size-exclusion chromatography and wavelength- and temperature-dependent CD. Thiazoline-containing RopC-U was a homogeneous dimer as judged by SEC (FIG. 29A-B) and retained a significant level of α-helical structure (FIG. 12E). It also melted cooperatively and reversibly with a T_{M} value of 27° C., a value almost identical to that of RopC itself (28° C.) (FIG. 12F). Thiazole-containing RopC-Z displayed more complex behavior; it was less homogeneous as judged by SEC and melted cooperatively (T_{M}=24° C.) but only after a refolding step (FIG. 33). These results indicate that the MicD-F can post-translationally install a thiazoline within a backbone of a helical bundle protein, and that ArtGox can oxidize this substrate to install a fully aromatic thiazole unit.

### Computational Analysis of the Effects of Thiazoline/Thiazole Formation on Local Backbone Flexibility

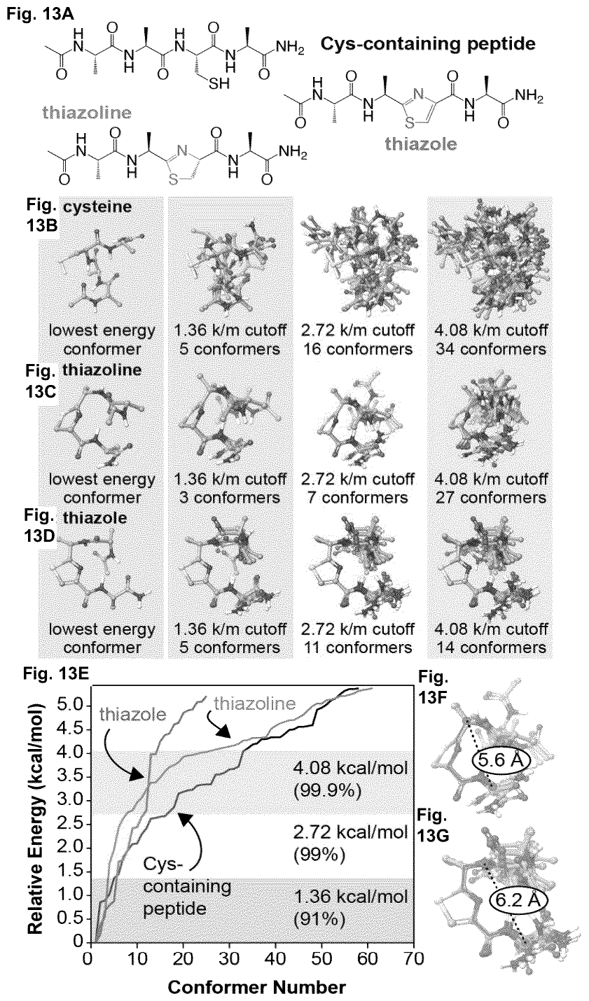

To explore the effects of the cyclization reaction on local backbone flexibility, we examined the conformational space of the tetrapeptide Ac-AACA-NH₂. The use of this simplified substrate allowed the inherent energetics of the backbone to be evaluated in the protein context without the complications of side chain fluctuations. Molecular mechanics methods (Macromodel, OPLS4 force field, implemented in Schrödinger Maestro software) were first used to generate and minimize large populations of conformers for cysteine-, thiazoline-, and thiazole-containing analogs (FIG. 13A). For each species, 10,000 starting structures were sampled using the Mixed Torsional/Low-Mode method. All conformers within 4 kcal/mol of each global minimum were then subjected to geometry optimization using DFT (Jaguar: B3LYP-D3/6-31G**). An SM8 method was used to determine the relative energies in aqueous media.51 All non-redundant conformers were then ranked based on these energies and compared.

The results of the conformational analysis appear in FIG. 13B-E, sorted by progressive energy cutoffs relative to each global minimum. The non-cyclized, cysteine-containing peptide exhibits the greatest flexibility, with 5 conformers being identified within 1.36 kcal/mol of the global minimum (91% of the population), and 16 within 2.72 kcal/mol (99%). Moreover, the identified conformers are largely non-superimposable, indicating that a high degree of conformational space is accessible within these energy ranges. In contrast, the thiazoline exhibits the most significant reduction in flexibility, with only 3 conformers identified at the 1.36 kcal/mol cutoff level and only 7 identified at a cut-off of 2.72 kcal/mol. Superposition of the thiazoline rings of these conformers reveals a rigid 6-bond motif that is preserved in all cases (FIG. 13F). The thiazole analog exhibits similarly reduced flexibility, with 11 conformers being identified within 2.72 kcal/mol of the global minimum. In this case, a rigid 7-bond motif can be identified (FIG. 13G). These evaluations provide the basis of models to predict the conformational effects of backbone cyclization on larger sequences and can be used to predict sequence locations in which cyclizations are more likely to be successful. We also disclose combining molecular dynamics studies with experimental data to examine the longer-range effects that result from introducing thiazoline and thiazole groups in larger peptides and full-size proteins. Such information can be used to apply this chemistry more generally to improve the physio-chemical properties of therapeutic proteins.

### CONCLUSIONS

One can imagine two mutually synergistic strategies to introduce non-natural monomers into polypeptide and protein oligomers.52 One “bottom-up” approach relies on extant or engineered ribosomes to accept and process tRNAs carrying diverse non-canonical α-amino or non-α-amino acids.53 Hundreds of non-canonical α-amino acids (as well as α-hydroxy acids54,55) have been introduced into proteins in cells and animals using genetic code expansion,56,57 which usually relies on novel orthogonal aminoacyl tRNA synthetases to generate the requisite acylated tRNAs. Select non-canonical α-amino acids58 and one β-amino acid30 have also been incorporated into proteins in vivo using endogenous α-aminoacyl tRNA synthetases. Alternatively, many non-canonical α-amino acids, as well as certain non-α-amino acids, including β-amino acids32,59 and certain polyketide precursors,33 can be introduced into short peptides in vitro and on small scale using genetic code reprogramming, in which a stoichiometric RNA co-reagent (Flexizyme60) generates the requisite acylated tRNA.

The second “top-down” approach is reminiscent of late-stage functionalization reactions used to manipulate complex small molecule natural products61,62 and the natural biosynthetic strategy used to assemble ribosomally synthesized and post-translationally modified peptides (RiPPs).13 In this approach, enzymes, chemical reagents, or chemical catalysts are employed to post-translationally modify a peptide12 or protein52 to install a new or modified monomer. Examples of this approach include reactions of natural or non-canonical protein side chains or modification of the N- or C-terminus.63-66 The only backbone-focused non-enzymatic reaction of which we are aware is the O-mesitylenesulfonylhydroxylamine-promoted oxidative elimination of Cys residues to generate a dehydroalanine backbone67 that is subsequently modified. We note that the top-down and bottom-up strategies are complementary, and both have the potential to operate in vivo where very high protein titers are possible.68

In this example we show that a constitutively active form of MicD and ArtGox, enzymes used in the biosynthesis of cyanobactin natural products69 are sufficiently promiscuous to process substrates containing diverse backbone-modified monomers within substrate polypeptides, even at positions immediately preceding or following the site of cyclization/dehydrogenation. The backbone-modified monomers compatible with MicD-F and ArtGox include many accepted by extant ribosomes in small-scale in vitro reactions, including aramids and β²- and β³-amino acids. The products of these reactions are sequence-defined chemical polymers with multiple, diverse, non-α-amino acid monomers. We show further that cyclodehydration and dehydrogenation can install thiazoline or thiazole backbones within protein loops and linkers without disrupting the native tertiary fold. Calculations reported here reveal the extent to which these heterocycles restrict conformational space; they also eliminate a peptide bond-both features could improve the stability or add function to linker sequences now commonplace in emerging biotherapeutics. Moreover, as thiazoles and thiazoline heterocycles are replete in natural products,5-7 small molecule drugs,8,9 and peptide-mimetic therapeutics,10 their installation in protein-based biotherapeutics can be used to improve or augment performance, activity, stability, and/or selectivity. More generally, this work represents a general strategy to expand the chemical diversity of the proteome without need for genetic manipulations.

### REFERENCES

(1) McGivern, J. G. Ziconotide: A Review of Its Pharmacology and Use in the Treatment of Pain. Neuropsychiatric Disease and Treatment 2007, 3(1), 69-85. https://doi.org/10.2147/nedt.2007.3.1.69.
(2) Schmidtko, A.; Lötsch, J.; Freynhagen, R.; Geisslinger, G. Ziconotide for Treatment of Severe Chronic Pain. The Lancet 2010,375(9725), 1569-1577. https://doi.org/10.1016/S0140-6736(10)60354-6.
(3) Arnison, P. G.; Bibb, M. J.; Bierbaum, G.; Bowers, A. A.; Bugni, T. S.; Bulaj, G.; Camarero, J. A.; Campopiano, D. J.; Challis, G. L.; Clardy, J.; Cotter, P. D.; Craik, D. J.; Dawson, M.; Dittmann, E.; Donadio, S.; Dorrestein, P. C.; Entian, K.-D.; Fischbach, M. A.; Garavelli, J. S.; Garansson, U.; Gruber, C. W.; Haft, D. H.; Hemscheidt, T. K.; Hertweck, C.; Hill, C.; Horswill, A. R.; Jaspars, M.; Kelly, W. L.; Klinman, J. P.; Kuipers, O. P.; Link, A. J.; Liu, W.; Marahiel, M. A.; Mitchell, D. A.; Moll, G. N.; Moore, B. S.; Müller, R.; Nair, S. K.; Nes, I. F.; Norris, G. E.; Olivera, B. M.; Onaka, H.; Patchett, M. L.; Piel, J.; Reaney, M. J. T.; Rebuffat, S.; Ross, R. P.; Sahl, H.-G.; Schmidt, E. W.; Selsted, M. E.; Severinov, K.; Shen, B.; Sivonen, K.; Smith, L.; Stein, T.; Sussmuth, R. D.; Tagg, J. R.; Tang, G.-L.; Truman, A. W.; Vederas, J. C.; Walsh, C. T.; Walton, J. D.; Wenzel, S. C.; Willey, J. M.; van der Donk, W. A. Ribosomally Synthesized and Post-Translationally Modified Peptide Natural Products: Overview and Recommendations for a Universal Nomenclature. Nat. Prod. Rep. 2013, 30(1), 108-160. https://doi.org/10.1039/C2NP20085F.
(4) Koehnke, J.; Bent, A. F.; Houssen, W. E.; Mann, G.; Jaspars, M.; Naismith, J. H. The Structural Biology of Patellamide Biosynthesis. Current Opinion in Structural Biology 2014, 29, 112-121. https://doi.org/10.1016/j.sbi.2014.10.006.
(5) Baumann, S.; Herrmann, J.; Raju, R.; Steinmetz, H.; Mohr, K. I.; Hüttel, S.; Harmrolfs, K.; Stadler, M.; Müller, R. Cystobactamids: Myxobacterial Topoisomerase Inhibitors Exhibiting Potent Antibacterial Activity. Angew. Chem. Int. Ed. 2014, 53(52), 14605-14609. https://doi.org/10.1002/anie.201409964.
(6) Groß, S.; Schnell, B.; Haack, P. A.; Auerbach, D.; Müller, R. In Vivo and in Vitro Reconstitution of Unique Key Steps in Cystobactamid Antibiotic Biosynthesis. Nat Commun 2021, 12(1), 1696. https://doi.org/10.1038/s41467-021-21848-3.
(7) Planke, T.; Cirnski, K.; Herrmann, J.; Müller, R.; Kirschning, A. Synthetic and Biological Studies on New Urea and Triazole Containing Cystobactamid Derivatives. Chem. Eur. J. 2020, 26(19), 4289-4296. https://doi.org/10.1002/chem.201904073.
(8) Taylor, R. D.; MacCoss, M.; Lawson, A. D. G. Rings in Drugs: Miniperspective. J. Med. Chem.2014, 57(14), 5845-5859. https://doi.org/10.1021/jm4017625.
(9) Vitaku, E.; Smith, D. T.; Njardarson, J. T. Analysis of the Structural Diversity, Substitution Patterns, and Frequency of Nitrogen Heterocycles among U.S. FDA Approved Pharmaceuticals: Miniperspective. J. Med. Chem.2014, 57(24), 10257-10274. https://doi.org/10.1021/jm501100b.
(10) Cabrele, C.; Martinek, T. A.; Reiser, O.; Berlicki, L. Peptides Containing β-Amino Acid Patterns: Challenges and Successes in Medicinal Chemistry. J. Med. Chem.2014, 57(23), 9718-9739. https://doi.org/10.1021/jm5010896.
(11) Ortega, M. A.; van der Donk, W. A. New Insights into the Biosynthetic Logic of Ribosomally Synthesized and Post-Translationally Modified Peptide Natural Products. Cell Chemical Biology 2016, 23(1), 31-44. https://doi.org/10.1016/j.chembiol.2015.11.012.
(12) Goto, Y.; Suga, H. Engineering of RiPP Pathways for the Production of Artificial Peptides Bearing Various Non-Proteinogenic Structures. Current Opinion in Chemical Biology 2018, 46, 82-90. https://doi.org/10.1016/j.cbpa.2018.06.014.
(13) Montalbán-López, M.; Scott, T. A.; Ramesh, S.; Rahman, I. R.; van Heel, A. J.; Viel, J. H.; Bandarian, V.; Dittmann, E.; Genilloud, O.; Goto, Y.; Grande Burgos, M. J.; Hill, C.; Kim, S.; Koehnke, J.; Latham, J. A.; Link, A. J.; Martinez, B.; Nair, S. K.; Nicolet, Y.; Rebuffat, S.; Sahl, H.-G.; Sareen, D.; Schmidt, E. W.; Schmitt, L.; Severinov, K.; Sussmuth, R. D.; Truman, A. W.; Wang, H.; Weng, J.-K.; van Wezel, G. P.; Zhang, Q.; Zhong, J.; Piel, J.; Mitchell, D. A.; Kuipers, O. P.; van der Donk, W. A. New Developments in RiPP Discovery, Enzymology and Engineering. Nat. Prod. Rep. 2021, 38(1), 130-239. https://doi.org/10.1039/D0NP00027B.
(14) Schmidt, E. W.; Nelson, J. T.; Rasko, D. A.; Sudek, S.; Eisen, J. A.; Haygood, M. G.; Ravel, J. Patellamide A and C Biosynthesis by a Microcin-like Pathway in Prochloron Didemni, the Cyanobacterial Symbiont of Lissoclinum Patella. Proceedings of the National Academy of Sciences 2005,102(20), 7315-7320. https://doi.org/10.1073/pnas.0501424102.
(15) Donia, M. S.; Ravel, J.; Schmidt, E. W. A Global Assembly Line for Cyanobactins. Nat Chem Biol 2008, 4(6), 341-343. https://doi.org/10.1038/nchembio.84.
(16) Tianero, Ma. D. B.; Donia, M. S.; Young, T. S.; Schultz, P. G.; Schmidt, E. W. Ribosomal Route to Small-Molecule Diversity. J. Am. Chem. Soc. 2012,134(1), 418-425. https://doi.org/10.1021/ja208278k.
(17) Koehnke, J.; Morawitz, F.; Bent, A. F.; Houssen, W. E.; Shirran, S. L.; Fuszard, M. A.; Smellie, I. A.; Botting, C. H.; Smith, M. C. M.; Jaspars, M.; Naismith, J. H. An Enzymatic Route to Selenazolines. ChemBioChem 2013, 14(5), 564-567. https://doi.org/10.1002/cbic.201300037.
(18) Goto, Y.; Suga, H. In Vitro Biosynthesis of Peptides Containing Exotic Azoline Analogues. ChemBioChem 2020, 21(1-2), 84-87. https://doi.org/10.1002/cbic.201900521.
(19) Kato, Y.; Kuroda, T.; Huang, Y.; Ohta, R.; Goto, Y.; Suga, H. Chemoenzymatic Posttranslational Modification Reactions for the Synthesis of Ψ[CH2NH]-Containing Peptides. Angew. Chem. Int. Ed. 2020, 59(2), 684-688. https://doi.org/10.1002/anie.201910894.
(20) Hudson, G. A.; Zhang, Z.; Tietz, J. I.; Mitchell, D. A.; van der Donk, W. A. In Vitro Biosynthesis of the Core Scaffold of the Thiopeptide Thiomuracin. J. Am. Chem. Soc. 2015,137(51), 16012-16015. https://doi.org/10.1021/jacs.5b10194.
(21) Fleming, S. R.; Bartges, T. E.; Vinogradov, A. A.; Kirkpatrick, C. L.; Goto, Y.; Suga, H.; Hicks, L. M.; Bowers, A. A. Flexizyme-Enabled Benchtop Biosynthesis of Thiopeptides. J. Am. Chem. Soc. 2019,141(2), 758-762. https://doi.org/10.1021/jacs.8b11521.
(22) Hayashi, S.; Ozaki, T.; Asamizu, S.; Ikeda, H.; Omura, S.; Oku, N.; Igarashi, Y.; Tomoda, H.; Onaka, H. Genome Mining Reveals a Minimum Gene Set for the Biosynthesis of 32-Membered Macrocyclic Thiopeptides Lactazoles. Chemistry & Biology 2014, 21(5), 679-688. https://doi.org/10.1016/j.chembiol.2014.03.008.
(23) Vinogradov, A. A.; Shimomura, M.; Goto, Y.; Ozaki, T.; Asamizu, S.; Sugai, Y.; Suga, H.; Onaka, H. Minimal Lactazole Scaffold for in Vitro Thiopeptide Bioengineering. Nat Commun 2020, 11(1), 2272. https://doi.org/10.1038/s41467-020-16145-4.
(24) Goto, Y.; Ito, Y.; Kato, Y.; Tsunoda, S.; Suga, H. One-Pot Synthesis of Azoline-Containing Peptides in a Cell-Free Translation System Integrated with a Posttranslational Cyclodehydratase. Chemistry & Biology 2014, 21(6), 766-774. https://doi.org/10.1016/j.chembiol.2014.04.008.
(25) Koehnke, J.; Mann, G.; Bent, A. F.; Ludewig, H.; Shirran, S.; Botting, C.; Lebl, T.; Houssen, W. E.; Jaspars, M.; Naismith, J. H. Structural Analysis of Leader Peptide Binding Enables Leader-Free Cyanobactin Processing. Nat Chem Biol 2015, 11(8), 558-563. https://doi.org/10.1038/nchembio.1841.
(26) Ge, Y.; Czekster, C. M.; Miller, O. K.; Botting, C. H.; Schwarz-Linek, U.; Naismith, J. H. Insights into the Mechanism of the Cyanobactin Heterocyclase Enzyme. Biochemistry 2019, 58(16), 2125-2132. https://doi.org/10.1021/acs.biochem.9b00084.
(27) Oueis, E.; Nardone, B.; Jaspars, M.; Westwood, N. J.; Naismith, J. H. Synthesis of Hybrid Cyclopeptides through Enzymatic Macrocyclization. ChemistryOpen 2017, 6(1), 11-14. https://doi.org/10.1002/open.201600134.
(28) Oueis, E.; Adamson, C.; Mann, G.; Ludewig, H.; Redpath, P.; Migaud, M.; Westwood, N. J.; Naismith, J. H. Derivatisable Cyanobactin Analogues: A Semisynthetic Approach. ChemBioChem 2015, 16(18), 2646-2650. https://doi.org/10.1002/cbic.201500494.
(29) Gao, S.; Ge, Y.; Bent, A. F.; Schwarz-Linek, U.; Naismith, J. H. Oxidation of the Cyanobactin Precursor Peptide Is Independent of the Leader Peptide and Operates in a Defined Order. Biochemistry 2018, 57(41), 5996-6002. https://doi.org/10.1021/acs.biochem.8b00835.
(30) Melo Czekster, C.; Robertson, W. E.; Walker, A. S.; Söll, D.; Schepartz, A. In Vivo Biosynthesis of a β-Amino Acid-Containing Protein. J. Am. Chem. Soc. 2016,138(16), 5194-5197. https://doi.org/10.1021/jacs.6b01023.
(31) Maini, R.; Chowdhury, S. R.; Dedkova, L. M.; Roy, B.; Daskalova, S. M.; Paul, R.; Chen, S.; Hecht, S. M. Protein Synthesis with Ribosomes Selected for the Incorporation of β-Amino Acids. Biochemistry 2015, 54(23), 3694-3706. https://doi.org/10.1021/acs.biochem.5b00389.
(32) Katoh, T.; Suga, H. Ribosomal Incorporation of Consecutive β-Amino Acids. J. Am. Chem. Soc. 2018,140(38), 12159-12167. https://doi.org/10.1021/jacs.8b07247.
(33) Ad, O.; Hoffman, K. S.; Cairns, A. G.; Featherston, A. L.; Miller, S. J.; Söll, D.; Schepartz, A. Translation of Diverse Aramid- and 1,3-Dicarbonyl-Peptides by Wild Type Ribosomesin Vitro. ACS Cent. Sci. 2019, 5(7), 1289-1294. https://doi.org/10.1021/acscentsci.9b00460.
(34) Lee, J.; Schwieter, K. E.; Watkins, A. M.; Kim, D. S.; Yu, H.; Schwarz, K. J.; Lim, J.; Coronado, J.; Byrom, M.; Anslyn, E. V.; Ellington, A. D.; Moore, J. S.; Jewett, M. C. Expanding the Limits of the Second Genetic Code with Ribozymes. Nat Commun 2019, 10(1), 5097. https://doi.org/10.1038/s41467-019-12916-w.
(35) Katoh, T.; Suga, H. Ribosomal Elongation of Aminobenzoic Acid Derivatives. J. Am. Chem. Soc. 2020, jacs.0c05765. https://doi.org/10.1021/jacs.0c05765.
(36) Chen, S.; Ji, X.; Gao, M.; Dedkova, L. M.; Hecht, S. M. In Cellulo Synthesis of Proteins Containing a Fluorescent Oxazole Amino Acid. J. Am. Chem. Soc. 2019,141(14), 5597-5601. https://doi.org/10.1021/jacs.8b12767.
(37) Ziemert, N.; Ishida, K.; Quillardet, P.; Bouchier, C.; Hertweck, C.; de Marsac, N. T.; Dittmann, E. Microcyclamide Biosynthesis in Two Strains of Microcystis Aeruginosa: From Structure to Genes and Vice Versa. AEM 2008, 74(6), 1791-1797. https://doi.org/10.1128/AEM.02392-07.
(38) Donia, M. S.; Schmidt, E. W. Linking Chemistry and Genetics in the Growing Cyanobactin Natural Products Family. Chemistry & Biology 2011, 18(4), 508-519. https://doi.org/10.1016/j.chembiol.2011.01.019.
(39) Gu, W.; Dong, S.-H.; Sarkar, S.; Nair, S. K.; Schmidt, E. W. The Biochemistry and Structural Biology of Cyanobactin Pathways: Enabling Combinatorial Biosynthesis. In Methods in Enzymology; Elsevier, 2018; Vol. 604, pp 113-163. https://doi.org/10.1016/bs.mie.2018.03.002.
(40) Tharp, J. M.; Ad, O.; Amikura, K.; Ward, F. R.; Garcia, E. M.; Cate, J. H. D.; Schepartz, A.; Söll, D. Initiation of Protein Synthesis with Non-Canonical Amino Acids In Vivo. Angewandte Chemie International Edition 2020, 59(8), 3122-3126. https://doi.org/10.1002/anie.201914671.
(41) Tharp, J. M.; Vargas-Rodriguez, O.; Schepartz, A.; Söll, D. Genetic Encoding of Three Distinct Noncanonical Amino Acids Using Reprogrammed Initiator and Nonsense Codons. ACS Chem. Biol. 2021, 16(4), 766-774. https://doi.org/10.1021/acschembio.1c00120.
(42) Cao, L.; Do, T.; Link, A. J. Mechanisms of Action of Ribosomally Synthesized and Posttranslationally Modified Peptides (RiPPs). Journal of Industrial Microbiology and Biotechnology 2021, 48(β-4), kuab005. https://doi.org/10.1093/jimb/kuab005.
(43) Chan, D. C. K.; Burrows, L. L. Thiopeptides: Antibiotics with Unique Chemical Structures and Diverse Biological Activities. J Antibiot 2021, 74(3), 161-175. https://doi.org/10.1038/s41429-020-00387-x.
(44) Sussmuth, R. D.; Mainz, A. Nonribosomal Peptide Synthesis-Principles and Prospects. Angew. Chem. Int. Ed. 2017, 56(14), 3770-3821. https://doi.org/10.1002/anie.201609079.
(45) Stas, M.; Broda, M. A.; Siodłak, D. Thiazole-Amino Acids: Influence of Thiazole Ring on Conformational Properties of Amino Acid Residues. Amino Acids 2021, 53(5), 673-686. https://doi.org/10.1007/s00726-021-02974-0.
(46) Shaner, N. C.; Campbell, R. E.; Steinbach, P. A.; Giepmans, B. N. G.; Palmer, A. E.; Tsien, R. Y. Improved Monomeric Red, Orange and Yellow Fluorescent Proteins Derived from Discosoma Sp. Red Fluorescent Protein. Nat Biotechnol 2004, 22(12), 1567-1572. https://doi.org/10.1038/nbt1037.
(47) Rana, M. S.; Wang, X.; Banerjee, A. An Improved Strategy for Fluorescent Tagging of Membrane Proteins for Overexpression and Purification in Mammalian Cells. Biochemistry 2018, 57(49), 6741-6751. https://doi.org/10.1021/acs.biochem.8b01070.
(48) Banner, D. W.; Kokkinidis, M.; Tsernoglou, D. Structure of the ColE1 Rop Protein at 1.7 Å Resolution. Journal of Molecular Biology 1987,196(3), 657-675. https://doi.org/10.1016/0022-2836(87)90039-8.
(49) Nagi, A. D.; Regan, L. An Inverse Correlation between Loop Length and Stability in a Four-Helix-Bundle Protein. Folding and Design 1997, 2(1), 67-75. https://doi.org/10.1016/S1359-0278(97)00007-2.
(50) Amprazi, M.; Kotsifaki, D.; Providaki, M.; Kapetaniou, E. G.; Fellas, G.; Kyriazidis, I.; Perez, J.; Kokkinidis, M. Structural Plasticity of 4—Helical Bundles Exemplified by the Puzzle-like Molecular Assembly of the Rop Protein. Proceedings of the National Academy of Sciences 2014,111(30), 11049-11054. https://doi.org/10.1073/pnas.1322065111.
(51) Marenich, A. V.; Cramer, C. J.; Truhlar, D. G. Universal Solvation Model Based on Solute Electron Density and on a Continuum Model of the Solvent Defined by the Bulk Dielectric Constant and Atomic Surface Tensions. J Phys Chem B2009,113(18), 6378-6396. https://doi.org/10.1021/jp810292n.
(52) Hoyt, E. A.; Cal, P. M. S. D.; Oliveira, B. L.; Bernardes, G. J. L. Contemporary Approaches to Site-Selective Protein Modification. Nature Reviews Chemistry 2019, 3(3), 147-171. https://doi.org/10.1038/s41570-019-0079-1.
(53) Noren, C.; Anthony-Cahill, S.; Griffith, M.; Schultz, P. A General Method for Site-Specific Incorporation of Unnatural Amino Acids into Proteins. Science 1989,244(4901), 182-188. https://doi.org/10.1126/science.2649980.
(54) Guo, J.; Wang, J.; Anderson, J. C.; Schultz, P. G. Addition of an α-Hydroxy Acid to the Genetic Code of Bacteria. Angew. Chem. Int. Ed. 2008, 47(4), 722-725. https://doi.org/10.1002/anie.200704074.
(55) England, P. M.; Zhang, Y.; Dougherty, D. A.; Lester, H. A. Backbone Mutations in Transmembrane Domains of a Ligand-Gated Ion Channel: Implications for the Mechanism of Gating. Cell 1999, 96(1), 89-98. https://doi.org/10.1016/s0092-8674(00)80962-9.
(56) Chin, J. W. Expanding and Reprogramming the Genetic Code. Nature 2017,550(7674), 53-60. https://doi.org/10.1038/nature24031.
(57) Young, D. D.; Schultz, P. G. Playing with the Molecules of Life. ACS Chem. Biol. 2018, 13(4), 854-870. https://doi.org/10.1021/acschembio.7b00974.
(58) Johnson, J.; Lu, Y.; Van Deventer, J.; Tirrell, D. Residue-Specific Incorporation of Non-Canonical Amino Acids into Proteins: Recent Developments and Applications. CURRENT OPINION IN CHEMICAL BIOLOGY 2010, 14(6), 774-780. https://doi.org/10.1016/j.cbpa.2010.09.013.
(59) Adaligil, E.; Song, A.; Hallenbeck, K. K.; Cunningham, C. N.; Fairbrother, W. J. Ribosomal Synthesis of Macrocyclic Peptides with B2- and B2,3-Homo-Amino Acids for the Development of Natural Product-Like Combinatorial Libraries. ACS Chem. Biol. 2021, 16(6), 1011-1018. https://doi.org/10.1021/acschembio.1c00062.
(60) Morimoto, J.; Hayashi, Y.; Iwasaki, K.; Suga, H. Flexizymes: Their Evolutionary History and the Origin of Catalytic Function. Acc. Chem. Res. 2011, 44(12), 1359-1368. https://doi.org/10.1021/ar2000953.
(61) Blakemore, D.; Castro, L.; Churcher, I.; Rees, D.; Thomas, A.; Wilson, D.; Wood, A. Organic Synthesis Provides Opportunities to Transform Drug Discovery. NATURE CHEMISTRY 2018, 10(4), 383-394. https://doi.org/10.1038/s41557-018-0021-z.
(62) Bostrom, J.; Brown, D.; Young, R.; Keseru, G. Expanding the Medicinal Chemistry Synthetic Toolbox. NATURE REVIEWS DRUG DISCOVERY 2018, 17(10), 709-727. https://doi.org/10.1038/nrd.2018.116.
(63) Bloom, S.; Liu, C.; Kölmel, D. K.; Qiao, J. X.; Zhang, Y.; Poss, M. A.; Ewing, W. R.; MacMillan, D. W. C. Decarboxylative Alkylation for Site-Selective Bioconjugation of Native Proteins via Oxidation Potentials. Nature Chemistry 2018, 10(2), 205-211. https://doi.org/10.1038/nchem.2888.
(64) Lobba, M. J.; Fellmann, C.; Marmelstein, A. M.; Maza, J. C.; Kissman, E. N.; Robinson, S. A.; Staahl, B. T.; Urnes, C.; Lew, R. J.; Mogilevsky, C. S.; Doudna, J. A.; Francis, M. B. Site-Specific Bioconjugation through Enzyme-Catalyzed Tyrosine-Cysteine Bond Formation. ACS Cent. Sci. 2020, 6(9), 1564-1571. https://doi.org/10.1021/acscentsci.0c00940.
(65) Rosen, C. B.; Francis, M. B. Targeting the N Terminus for Site-Selective Protein Modification. Nature Chemical Biology 2017, 13(7), 697-705. https://doi.org/10.1038/nchembio.2416.
(66) Marmelstein, A. M.; Lobba, M. J.; Mogilevsky, C. S.; Maza, J. C.; Brauer, D. D.; Francis, M. B. Tyrosinase-Mediated Oxidative Coupling of Tyrosine Tags on Peptides and Proteins. J. Am. Chem. Soc. 2020,142(11), 5078-5086. https://doi.org/10.1021/jacs.9b12002.
(67) Bernardes, G.; Chalker, J.; Errey, J.; Davis, B. Facile Conversion of Cysteine and Alkyl Cysteines to Dehydroalanine on Protein Surfaces: Versatile and Switchable Access to Functionalized Proteins. JOURNAL OF THE AMERICAN CHEMICAL SOCIETY 2008,130(15), 5052-+. https://doi.org/10.1021/ja800800p.
(68) Santiago, S.; Ad, O.; Shah, B.; Zhang, Z.; Zhang, X.; Chatterjee, A.; Schepartz, A. Genetic Code Expansion in the Engineered Organism Vmax X2: High Yield and Exceptional Fidelity. bioRxiv2021, 2021.06.22.449487. https://doi.org/10.1101/2021.06.22.449487.
(69) Czekster, C.; Ge, Y.; Naismith, J. Mechanisms of Cyanobactin Biosynthesis. CURRENT OPINION IN CHEMICAL BIOLOGY 2016, 35, 80-88. https://doi.org/10.1016/j.cbpa.2016.08.029.

### Supplemental Materials

### Enzyme Expression, Purification, and Characterization

### MicD-F Expression Plasmid and Translation Product

The plasmid used to express MicD-F (pJExpress411-MicD-F) was graciously provided by Professor James Naismith (University of Oxford).2 The translation product encoded by this plasmid is the full sequence of MicD (heterocyclase from Microcystis aeruginosa, Uniprot ID: A8Y998) preceded by five repeats of a Gly-Ala spacer, residues Thr18 to Ala37 from PatE (Uniprot ID: A0MH79), a TEV protease recognition site, and an N-terminal 6×His purification tag. The full sequence of the translation product is provided below.

SEQ ID NO: 7
MSHHHHHHDYDENLYFQGSRLTAGQLSSQLAELSEEALGDAGAGAGAGA
GAKLMQSTPLLQIQPHFHVEVIEPKQVYLLGEQANYALTGQLYCQILPL
LDGQHSREQIVEKLDGEVPSEYIDYVLDRLAEKGYLTEAAPELSSEVAA
FWSELGIAPPVAAEALRQSVTLTPVGNISEVTVAALTTALRDIGISVQT
PTEAGSPTALNVVLTDDYLQPELAKINKQALESQQTWLLVKPVGSVLWL
GPVFVPGKTGCWDCLAHRLRGNREVEASVLQQKQAQQQRNGQSGSVIGC
LPTARATLPSTLQTGLQFAATEIAKWIVKHHVKATAPGTVFFPTLDGKI
ITFNHTVIDLKSHVLVRRSQCPSCGDRQILHRQGFEPVKLVSRRKHFTH
DGGHRAFTPEQTVQKYQHLVSPITGVVTELVRLTDPANPLVHTYKAGHA
FGSATTLRGLRNTLKYKSSGKGKTDIQSRASGLCEAIERYSGIFQGDEP
RKRATLAELGDLALHPESLLYFSNTQYANREELNAQGSAAAYRWIPNRF
DVSQAIDWTPVWSLTEQKHKYVPTAFCYYGYPLPEEQRFCKADSNGNAA
GNTLEEAILQGFLELVERDSIAMWWYNRIRRPAVDLSTFDEPYFVDLQQ
FYQQQNRELWVLDVTADLGIPAFAGFSRRTVGTSERISIGFGAHLDPTI
AILRALTEVSQVGLELDKIPDDKLDGESKDWMLNVTVENHPWLAPDPSV
PMKTASDYPKRWSDDIHTDVMNCVKTAQTAGLEVMVLDQTRPDIGLNVV
KVIIPGMRTFWTRFGQGRLYDIPVKLGWLDAPLAEEELNQTNIPF

### MicD-F Expression and Purification

A starter culture of 5 mL of Miller's LB Broth (AmericanBio, catalog #AB01201) containing 50 μg/mL kanamycin was inoculated with a single colony of E. coli BL21 (DE3) harboring the pJExpress411-MicD-F plasmid and grown overnight at 37° C. with shaking at 200 rpm. The starter culture (5 mL) was used to inoculate a 500 mL expression culture of Miller's LB Broth which also contained 50 μg/mL kanamycin. The expression culture was grown at 37° C. with shaking at 200 rpm to an OD₆₀₀ of 0.6 at which point the expression culture was induced with 1 mM IPTG, transferred to a 20° C. incubator, and grown for 24 hours with shaking at 200 rpm. The expression culture was harvested by centrifugation at 4,300×g at 4° C. for 20 minutes. The resulting cell pellet was suspended in 10 mL of Lysis Buffer (20 mM Tris-HCl, 500 mM NaCl, pH 8.0) containing 1 tablet of cOmplete, mini EDTA-free ULTRA protease inhibitor cocktail (Sigma-Aldrich, St. Louis, MO). The cell suspension was disrupted by sonication on ice (Branson Sonifier 250, 3 cycles of 30 second pulse at 30% duty cycle and microtip limit of 5 followed by 60 second pause). The cell lysate was cleared by centrifugation at 4,300×g at 4° C. for 20 minutes. A gravity flow Poly-Prep Chromatography Column (Bio-Rad Laboratories, Hercules, CA) was loaded with 2 mL of packed Ni-NTA agarose (Qiagen, Germantown, MD) and equilibrated with 10 mL of Lysis Buffer. The 6×His-tagged protein was bound to the Ni-NTA column by passing the cleared cell lysate over the column three times. Non-specifically bound proteins were removed by washing the Ni-NTA column with 10 mL of Wash Buffer (20 mM Tris-HCl, 500 mM NaCl, 50 mM imidazole pH 8.0). The 6×His-tagged protein was eluted by washing the Ni-NTA column with 5 mL of Elution Buffer (20 mM Tris-HCl, 500 mM NaCl, 250 mM imidazole pH 8.0). The purified protein was concentrated and exchanged into Storage Buffer (20 mM HEPES, 150 mM NaCl, 1 mM TCEP, pH 7.4) using Amicon Ultra-0.5 mL centrifugal filters (MilliporeSigma, Burlington, MA) with a 30 kDa molecular weight cut off according to the manufacturer's instructions. The purified protein was quantified using absorbance at 280 nm (molar extinction coefficient of 125,250 M⁻¹ cm⁻¹), diluted to 500 M using Storage Buffer, snap frozen as single-use aliquots, and stored at −80° C. The typical expression yield of MicD-F using the above protocol was 30 mg/L of E. coli culture.

### ArtGox Expression Plasmid and Translation Product

The plasmid used to express ArtGox (pEHISTEVSUMO-ArtGox) was generously provided by Professor James Naismith (University of Oxford).3,4 The translation product encoded by this plasmid is the oxidase domain of ArtG (thiazoline oxidase from Arthrospira platensis, Uniprot ID: H1W8K1) preceded by a TEV protease recognition site, a SUMO fusion tag, and an N-terminal 6×His purification tag. The full sequence of the translation product is provided below.

SEQ ID NO: 8
MGSSHHHHHHGSDSEVNQEAKPEVKPEVKPETHINLKVSDGSSEIFFKI
KKTTPLRRLMEAFAKRQGKEMDSLRFLYDGIRIQADQTPEDLDMEDNDI
IEAHREQIGGDIPTTENLYFQGAMGRTYPFAVSLNSTIQVSTTADGYAI
SPANTDPGQSIAPSMVTLPAITGMGDALAHLQAGTATLQQLTQTLSARE
GVEAGEQLAATLQQMGDRGWLQYAVLPLAIAEPMVESAELDLNSPHWTQ
AKVSLSRFAYQRSHAGGMVLESPLSKFRVKLLDWRSSAILAQLAQPQPL
GWVTPPPQIGAETAYQFLNLLWATGFLTVETEAPELKLWEFHNLLFHSR
CRQGRHDYPTGDIAASLDIWDEFPVVKPPMSGHIVPLPQLSIDAIRQRD
KTLTTAIEKRASIREYDENHPITIEQLGELLYRTARIKEIYTHDAEQAE
LLKAQFGEDFDWGELSRRPYPCGGAMYELEIYLAVRRCAGVKPGLYHYD
PLNHQLAQIDAADADIQALLKDAHQSSGEQGMPQVLLMITARFGRLFRK
YRSLAYALVLKHVGVLYQNLYLVATNMGLAPCALGAGDSDRFAQATGLD
YVVESSVGEFMLGSL

### ArtGox Expression and Purification

A starter culture of 5 mL of Miller's LB Broth (AmericanBio, catalog #AB01201) supplemented with 50 μg/mL kanamycin was inoculated with a single colony of E. coli BL21 (DE3) harboring the pEHISTEVSUMO-ArtGox plasmid and grown overnight at 37° C. with shaking at 200 rpm. The starter culture (5 mL) was used to inoculate a 500 mL expression culture of Miller's LB Broth supplemented with 50 μM riboflavin and containing 50 μg/mL kanamycin. The expression culture was grown at 37° C. with shaking at 200 rpm to an OD₆₀₀ of 0.6 at which point it was induced with 1 mM IPTG, transferred to a 20° C. incubator, and grown for 24 hours with shaking at 200 rpm. The expression culture was harvested by centrifugation at 4,300×g at 4° C. for 20 minutes. The resulting cell pellet was suspended in 10 mL of Lysis Buffer (20 mM Tris-HCl, 500 mM NaCl, 50 μM flavin mononucleotide, pH 8.0) containing 1 tablet of cOmplete, mini EDTA-free ULTRA protease inhibitor cocktail (Sigma-Aldrich, St. Louis, MO). The cell suspension was disrupted by sonication on ice (Branson Sonifier 250, 3 cycles of 30 second pulse at 30% duty cycle and microtip limit of 5 followed by 60 second pause). The cell lysate was cleared by centrifugation at 4,300×g at 4° C. for 20 minutes. A gravity flow Poly-Prep Chromatography Column (Bio-Rad Laboratories, Hercules, CA) was loaded with 2 mL of packed Ni-NTA agarose (Qiagen, Germantown, MD) and equilibrated with 10 mL of Lysis Buffer. The 6×His-tagged protein was bound to the Ni-NTA column by passing the cleared cell lysate over the column three times. Non-specifically bound proteins were removed by washing the Ni-NTA column with 10 mL of Wash Buffer (20 mM Tris-HCl, 500 mM NaCl, 50 mM imidazole, 50 M flavin mononucleotide, pH 8.0). The 6×His-tagged protein was eluted by washing the Ni-NTA column with 5 mL of Elution Buffer (20 mM Tris-HCl, 500 mM NaCl, 250 mM imidazole, 50 M flavin mononucleotide, pH 8.0). The purified protein was concentrated and exchanged into Storage Buffer (20 mM HEPES, 150 mM NaCl, 1 mM TCEP, pH 7.4) using Amicon Ultra-0.5 mL centrifugal filters (MilliporeSigma, Burlington, MA) with a 30 kDa molecular weight cut off according to the manufacturer's instructions. The purified protein was quantified using absorbance at 280 nm (molar extinction coefficient of 75,290 M⁻¹ cm⁻¹), diluted to 800 M using Storage Buffer, snap frozen as single use aliquots, and stored at −80° C. The typical expression yield of ArtGox using the above protocol was 40 mg/L of E. coli culture.

### Characterization of MicD-F and ArtGox

SDS-PAGE. Purified samples of MicD-F and ArtGox (4 g) were mixed (4:1) with SDS-PAGE sample buffer (5% β-Mercaptoethanol, 0.02% bromophenol blue, 30% glycerol, 10% SDS, 250 mM Tris-HCl, pH 6.8). The protein samples were reduced and denatured by incubation at 95° C. for 5 minutes. Reduced and denatured samples were separated using a 4-15% mini-PROTEAN TGX gel (Bio-Rad Laboratories, Hercules, CA) run at 120 V for 60 minutes in Tris-Glycine-SDS running buffer (3 g/L tris, 14.4 g/L glycine, 1 g/L sodium dodecyl sulfate, pH 8.3) and their molecular weights compared against Precision Plus Protein Dual Color Standards (Bio-Rad Laboratories, Hercules, CA). Protein content was visualized using Coomassie stain (1 g/L Coomassie Brilliant Blue in methanol:water:acetic acid (5:4:1)) and imaged using a Bio-Rad Chemidoc MP Imaging System. Band intensities were quantified using the gel analysis tool of FIJI.5 Please see FIG. 14 for SDS-PAGE and LC-MS characterization of MicD-F and ArtGox.

Cloning, Expression, Purification, and Characterization of mCherry and Rop Variants
Design of mCherry Variants

C-terminally modified substrates (mCherryC+ and mCherryC−) are variants of the mCherry sequence associated with Uniprot ID: X5DSL3. Both contained an N-terminal 6×His purification tag, the natural three-residue chromophore-forming sequence MYG, and a C-terminal extension that included a TEV protease recognition site, a MicD-F/ArtGox compatible substrate, and a FLAG purification tag. Internally-modified substrates (mCherry137+, mCherry174+, mCherry192+ and mCherry211+) are also variants of the mCherry sequence associated with Uniprot ID: X5DSL3. Each contained an N-terminal FLAG purification tag, the natural three-residue chromophore-forming sequence MYG, and a C-terminal 6×His purification tag. A MicD-F/ArtGox compatible substrate sequence was inserted internally on the C-terminal side of the indicated residue (137, 174, 192, or 211). The full sequences of the mCherry translation products are provided below.

mCherryC+
SEQ ID NO: 9
MHHHHHHMVSKGEEDNMAIIKEFMRFKVHMEGSVNGHEFEIEGEGEGRP
YEGTQTAKLKVTKGGPLPFAWDILSPQFMYGSKAYVKHPADIPDYLKLS
FPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAK
KPVQLPGAYNVNIKLDITSHNEDYTIVEQYERAEGRHSTGGMDELYKEN
LYFQGMCAYDGDYKDDDDK
mCherryC-
SEQ ID NO: 10
MHHHHHHMVSKGEEDNMAIIKEFMRFKVHMEGSVNGHEFEIEGEGEGRP
YEGTQTAKLKVTKGGPLPFAWDILSPQFMYGSKAYVKHPADIPDYLKLS
FPEGFKWERVMNFEDGGVVTVTQDSSLQDGEFIYKVKLRGTNFPSDGPV
MQKKTMGWEASSERMYPEDGALKGEIKQRLKLKDGGHYDAEVKTTYKAK
KPVQLPGAYNVNIKLDITSHNEDYTIVEQYERAEGRHSTGGMDELYKEN
LYFQGMAAYDGDYKDDDDK
mCherry137+
SEQ ID NO: 11
MDYKDDDDKMVSKGEEDNMAIIKEFMRFKVHMEGSVNGHEFEIEGEGEG
RPYEGTQTAKLKVTKGGPLPFAWDILSPQFMYGSKAYVKHPADIPDYLK
LSFPEGFKWERVMNFEDGGVVTVTQDSSLQDGEFIYKVKLRGTNFPSDM
CAYDGGPVMQKKTMGWEASSERMYPEDGALKGEIKQRLKLKDGGHYDAE
VKTTYKAKKPVQLPGAYNVNIKLDITSHNEDYTIVEQYERAEGRHSTGG
MDELYKHHHHHH
mCherry174+
SEQ ID NO: 12
MDYKDDDDKMVSKGEEDNMAIIKEFMRFKVHMEGSVNGHEFEIEGEGEG
RPYEGTQTAKLKVTKGGPLPFAWDILSPQFMYGSKAYVKHPADIPDYLK
LSFPEGFKWERVMNFEDGGVVTVTQDSSLQDGEFIYKVKLRGTNFPSDG
PVMQKKTMGWEASSERMYPEDGALKGEIKQRLKLKDMCAYDGGGHYDAE
VKTTYKAKKPVQLPGAYNVNIKLDITSHNEDYTIVEQYERAEGRHSTGG
MDELYKHHHHHH
mCherry192+
SEQ ID NO: 13
MDYKDDDDKMVSKGEEDNMAIIKEFMRFKVHMEGSVNGHEFEIEGEGEG
RPYEGTQTAKLKVTKGGPLPFAWDILSPQFMYGSKAYVKHPADIPDYLK
LSFPEGFKWERVMNFEDGGVVTVTQDSSLQDGEFIYKVKLRGTNFPSDG
PVMQKKTMGWEASSERMYPEDGALKGEIKQRLKLKDGGHYDAEVKTTYK
AKKPVMCAYDGQLPGAYNVNIKLDITSHNEDYTIVEQYERAEGRHSTGG
MDELYKHHHHHH
mCherry211+
SEQ ID NO: 14
MDYKDDDDKMVSKGEEDNMAIIKEFMRFKVHMEGSVNGHEFEIEGEGEG
RPYEGTQTAKLKVTKGGPLPFAWDILSPQFMYGSKAYVKHPADIPDYLK
LSFPEGFKWERVMNFEDGGVVTVTQDSSLQDGEFIYKVKLRGTNFPSDG
PVMQKKTMGWEASSERMYPEDGALKGEIKQRLKLKDGGHYDAEVKTTYK
AKKPVQLPGAYNVNIKLDITSHNEMCAYDGDYTIVEQYERAEGRHSTGG
MDELYKHHHHHH

Cloning mCherry Constructs into pET-32a(+) Vector

The sequences encoding mCherryC+, mCherryC−, mCherry137+, mCherry174+, mCherry192+ and mCherry211+ were cloned into a pET-32a(+) plasmid as follows. Circular pET-32a(+) vector (1 g, Millipore Sigma, catalog #69015-3) was incubated with 1 L each of restrictions enzymes NdeI (New England Biosciences, catalog #R0111S) and NotI-HF (New England Biosciences, catalog #R3189S) in cutSmart Buffer (New England Biolabs, catalog #B7204S) at 37° C. for 1 hour. The entire restriction digest reaction was run on a 0.8% agarose gel at 150V for 45 minutes and linear 5.4 kbp and 0.5 kbp fragments were observed using a blue light transilluminator. The larger 5.4 kbp fragment was excised from the gel and purified using a Monarch DNA Gel Extraction Kit (NEB, catalog #T1020S). The concentration of purified, linearized pET-32(a)+ was determined by absorbance at 260 nm. Next, 33.3 ng of purified, linearized pET-32(a)+ and 100 ng of respective gBlock DNA fragment (Integrated DNA Technologies, Coralville, IA) encoding mCherry constructs were combined in a 10 L Gibson Assembly reaction6 containing HiFI DNA Assembly Master Mix (NEB, catalog #E2621L) and incubated at 50° C. for 1 hour to generate circular pET-32(a)+ vectors containing coding sequences for mCherry constructs. Circularized plasmids from the previous step were transformed into NEB 5-alpha competent E. coli (NEB, catalog #C2987H) as follows. Frozen stocks of cells were thawed on ice for 10 minutes. Upon thawing 4 L of the previous Gibson Assembly reaction was added to cells and incubated on ice for 30 minutes. Cells incubated with plasmid were then subjected to heat shock at 42° C. for 30 seconds and placed on ice for 5 minutes. 900 L of SOC outgrowth medium (NEB, catalog #B9020S) was added to cells and cells were incubated at 37° C. for 1 hour with shaking at 200 rpm. Agar plates containing 100 g/mL carbenicillin were inoculated with 100 L of transformed cells and grown overnight at 37° C. 5 single colonies per construct were picked and inoculated into liquid cultures containing 5 mL LB+100 μg/mL carbenicillin and grown for 16 hours at 37° C. Pure plasmid was isolated from 5 mL cultures using Qiaprep Spin Miniprep Kit (Qiagen, catalog #27106) and sequences were confirmed by Sanger sequencing at the UC Berkeley DNA Sequencing Facility. Plasmids containing the precise coding sequence for each construct were transformed into chemically competent BL21 E. coli (NEB, catalog #C2530H) following the same transformation protocol detailed above for large scale protein expression.

mCherryC+ gBlock Sequence
SEQ ID NO: 15
TCCCCTCTAGAAATAATTTTGTTTAACTTTAAGAAGGAGATATACATATGCACCACC
ATCACCATCATATGGTGAGCAAGGGCGAGGAGGATAACATGGCCATCATCAAGGA
GTTCATGCGCTTCAAGGTGCACATGGAGGGCTCCGTGAACGGCCACGAGTTCGAGA
TCGAGGGCGAGGGCGAGGGCCGCCCCTACGAGGGCACCCAGACCGCCAAGCTGAA
GGTGACCAAGGGTGGCCCCCTGCCCTTCGCCTGGGACATCCTGTCCCCTCAGTTCAT
GTACGGCTCCAAGGCCTACGTGAAGCACCCCGCCGACATCCCCGACTACTTGAAGC
TGTCCTTCCCCGAGGGCTTCAAGTGGGAGCGCGTGATGAACTTCGAGGACGGCGGC
GTGGTGACCGTGACCCAGGACTCCTCCCTGCAGGACGGCGAGTTCATCTACAAGGT
GAAGCTGCGCGGCACCAACTTCCCCTCCGACGGCCCCGTAATGCAGAAGAAGACCA
TGGGCTGGGAGGCCTCCTCCGAGCGGATGTACCCCGAGGACGGCGCCCTGAAGGGC
GAGATCAAGCAGAGGCTGAAGCTGAAGGACGGCGGCCACTACGACGCTGAGGTCA
AGACCACCTACAAGGCCAAGAAGCCCGTGCAGCTGCCCGGCGCCTACAACGTCAAC
ATCAAGTTGGACATCACCTCCCACAACGAGGACTACACCATCGTGGAACAGTACGA
ACGCGCCGAGGGCCGCCACTCCACCGGCGGCATGGACGAGCTGTACAAGGAAAAT
CTTTATTTTCAGGGAATGTGCGCCTACGACGGAGACTACAAAGACGACGACGACAA
ATAAGCGGCCGCACTCGAGCACC
mCherryC− gBlock Sequence
SEQ ID NO: 16
TCCCCTCTAGAAATAATTTTGTTTAACTTTAAGAAGGAGATATACATATGCACCACC
ATCACCATCATATGGTGAGCAAGGGCGAGGAGGATAACATGGCCATCATCAAGGA
GTTCATGCGCTTCAAGGTGCACATGGAGGGCTCCGTGAACGGCCACGAGTTCGAGA
TCGAGGGCGAGGGCGAGGGCCGCCCCTACGAGGGCACCCAGACCGCCAAGCTGAA
GGTGACCAAGGGTGGCCCCCTGCCCTTCGCCTGGGACATCCTGTCCCCTCAGTTCAT
GTACGGCTCCAAGGCCTACGTGAAGCACCCCGCCGACATCCCCGACTACTTGAAGC
TGTCCTTCCCCGAGGGCTTCAAGTGGGAGCGCGTGATGAACTTCGAGGACGGCGGC
GTGGTGACCGTGACCCAGGACTCCTCCCTGCAGGACGGCGAGTTCATCTACAAGGT
GAAGCTGCGCGGCACCAACTTCCCCTCCGACGGCCCCGTAATGCAGAAGAAGACCA
TGGGCTGGGAGGCCTCCTCCGAGCGGATGTACCCCGAGGACGGCGCCCTGAAGGGC
GAGATCAAGCAGAGGCTGAAGCTGAAGGACGGCGGCCACTACGACGCTGAGGTCA
AGACCACCTACAAGGCCAAGAAGCCCGTGCAGCTGCCCGGCGCCTACAACGTCAAC
ATCAAGTTGGACATCACCTCCCACAACGAGGACTACACCATCGTGGAACAGTACGA
ACGCGCCGAGGGCCGCCACTCCACCGGCGGCATGGACGAGCTGTACAAGGAAAAT
CTTTATTTTCAGGGAATGGCCGCCTACGACGGAGACTACAAAGACGACGACGACAA
ATAAGCGGCCGCACTCGAGCACC
mCherry137+ gBlock Sequence
SEQ ID NO: 17
TCCCCTCTAGAAATAATTTTGTTTAACTTTAAGAAGGAGATATACATATGGACTACA
AAGACGACGACGACAAAATGGTGAGCAAGGGCGAGGAGGATAACATGGCCATCAT
CAAGGAGTTCATGCGCTTCAAGGTGCACATGGAGGGCTCCGTGAACGGCCACGAGT
TCGAGATCGAGGGCGAGGGCGAGGGCCGCCCCTACGAGGGCACCCAGACCGCCAA
GCTGAAGGTGACCAAGGGTGGCCCCCTGCCCTTCGCCTGGGACATCCTGTCCCCTCA
GTTCATGTACGGCTCCAAGGCCTACGTGAAGCACCCCGCCGACATCCCCGACTACTT
GAAGCTGTCCTTCCCCGAGGGCTTCAAGTGGGAGCGCGTGATGAACTTCGAGGACG
GCGGCGTGGTGACCGTGACCCAGGACTCCTCCCTGCAGGACGGCGAGTTCATCTAC
AAGGTGAAGCTGCGCGGCACCAACTTCCCCTCCGACATGTGCGCCTACGACGGAGG
CCCCGTAATGCAGAAGAAGACCATGGGCTGGGAGGCCTCCTCCGAGCGGATGTACC
CCGAGGACGGCGCCCTGAAGGGCGAGATCAAGCAGAGGCTGAAGCTGAAGGACGG
CGGCCACTACGACGCTGAGGTCAAGACCACCTACAAGGCCAAGAAGCCCGTGCAGC
TGCCCGGCGCCTACAACGTCAACATCAAGTTGGACATCACCTCCCACAACGAGGAC
TACACCATCGTGGAACAGTACGAACGCGCCGAGGGCCGCCACTCCACCGGCGGCAT
GGACGAGCTGTACAAGCACCACCATCACCATCATTAAGCGGCCGCACTCGAGCACC
mCherry174+ gBlock Sequence
SEQ ID NO: 18
TCCCCTCTAGAAATAATTTTGTTTAACTTTAAGAAGGAGATATACATATGGACTACA
AAGACGACGACGACAAAATGGTGAGCAAGGGCGAGGAGGATAACATGGCCATCAT
CAAGGAGTTCATGCGCTTCAAGGTGCACATGGAGGGCTCCGTGAACGGCCACGAGT
TCGAGATCGAGGGCGAGGGCGAGGGCCGCCCCTACGAGGGCACCCAGACCGCCAA
GCTGAAGGTGACCAAGGGTGGCCCCCTGCCCTTCGCCTGGGACATCCTGTCCCCTCA
GTTCATGTACGGCTCCAAGGCCTACGTGAAGCACCCCGCCGACATCCCCGACTACTT
GAAGCTGTCCTTCCCCGAGGGCTTCAAGTGGGAGCGCGTGATGAACTTCGAGGACG
GCGGCGTGGTGACCGTGACCCAGGACTCCTCCCTGCAGGACGGCGAGTTCATCTAC
AAGGTGAAGCTGCGCGGCACCAACTTCCCCTCCGACGGCCCCGTAATGCAGAAGAA
GACCATGGGCTGGGAGGCCTCCTCCGAGCGGATGTACCCCGAGGACGGCGCCCTGA
AGGGCGAGATCAAGCAGAGGCTGAAGCTGAAGGACATGTGCGCCTACGACGGAGG
CGGCCACTACGACGCTGAGGTCAAGACCACCTACAAGGCCAAGAAGCCCGTGCAGC
TGCCCGGCGCCTACAACGTCAACATCAAGTTGGACATCACCTCCCACAACGAGGAC
TACACCATCGTGGAACAGTACGAACGCGCCGAGGGCCGCCACTCCACCGGCGGCAT
GGACGAGCTGTACAAGCACCACCATCACCATCATTAAGCGGCCGCACTCGAGCACC
mCherry192+ gBlock Sequence
SEQ ID NO: 19
TCCCCTCTAGAAATAATTTTGTTTAACTTTAAGAAGGAGATATACATATGGACTACA
AAGACGACGACGACAAAATGGTGAGCAAGGGCGAGGAGGATAACATGGCCATCAT
CAAGGAGTTCATGCGCTTCAAGGTGCACATGGAGGGCTCCGTGAACGGCCACGAGT
TCGAGATCGAGGGCGAGGGCGAGGGCCGCCCCTACGAGGGCACCCAGACCGCCAA
GCTGAAGGTGACCAAGGGTGGCCCCCTGCCCTTCGCCTGGGACATCCTGTCCCCTCA
GTTCATGTACGGCTCCAAGGCCTACGTGAAGCACCCCGCCGACATCCCCGACTACTT
GAAGCTGTCCTTCCCCGAGGGCTTCAAGTGGGAGCGCGTGATGAACTTCGAGGACG
GCGGCGTGGTGACCGTGACCCAGGACTCCTCCCTGCAGGACGGCGAGTTCATCTAC
AAGGTGAAGCTGCGCGGCACCAACTTCCCCTCCGACGGCCCCGTAATGCAGAAGAA
GACCATGGGCTGGGAGGCCTCCTCCGAGCGGATGTACCCCGAGGACGGCGCCCTGA
AGGGCGAGATCAAGCAGAGGCTGAAGCTGAAGGACGGCGGCCACTACGACGCTGA
GGTCAAGACCACCTACAAGGCCAAGAAGCCCGTGATGTGCGCCTACGACGGACAGC
TGCCCGGCGCCTACAACGTCAACATCAAGTTGGACATCACCTCCCACAACGAGGAC
TACACCATCGTGGAACAGTACGAACGCGCCGAGGGCCGCCACTCCACCGGCGGCAT
GGACGAGCTGTACAAGCACCACCATCACCATCATTAAGCGGCCGCACTCGAGCACC
mCherry211+ gBlock Sequence
SEQ ID NO: 20
TCCCCTCTAGAAATAATTTTGTTTAACTTTAAGAAGGAGATATACATATGGACTACA
AAGACGACGACGACAAAATGGTGAGCAAGGGCGAGGAGGATAACATGGCCATCAT
CAAGGAGTTCATGCGCTTCAAGGTGCACATGGAGGGCTCCGTGAACGGCCACGAGT
TCGAGATCGAGGGCGAGGGCGAGGGCCGCCCCTACGAGGGCACCCAGACCGCCAA
GCTGAAGGTGACCAAGGGTGGCCCCCTGCCCTTCGCCTGGGACATCCTGTCCCCTCA
GTTCATGTACGGCTCCAAGGCCTACGTGAAGCACCCCGCCGACATCCCCGACTACTT
GAAGCTGTCCTTCCCCGAGGGCTTCAAGTGGGAGCGCGTGATGAACTTCGAGGACG
GCGGCGTGGTGACCGTGACCCAGGACTCCTCCCTGCAGGACGGCGAGTTCATCTAC
AAGGTGAAGCTGCGCGGCACCAACTTCCCCTCCGACGGCCCCGTAATGCAGAAGAA
GACCATGGGCTGGGAGGCCTCCTCCGAGCGGATGTACCCCGAGGACGGCGCCCTGA
AGGGCGAGATCAAGCAGAGGCTGAAGCTGAAGGACGGCGGCCACTACGACGCTGA
GGTCAAGACCACCTACAAGGCCAAGAAGCCCGTGCAGCTGCCCGGCGCCTACAACG
TCAACATCAAGTTGGACATCACCTCCCACAACGAGATGTGCGCCTACGACGGAGAC
TACACCATCGTGGAACAGTACGAACGCGCCGAGGGCCGCCACTCCACCGGCGGCAT
GGACGAGCTGTACAAGCACCACCATCACCATCATTAAGCGGCCGCACTCGAGCACC

### Design of Rop Variants

Loop-modified substrates (RopN, RopC, RopM, RopCG4) are analogs of the Rop sequence associated with Uniprot ID: P03051. All contained an N-terminal FLAG purification tag, and one of five ten-amino acid sequences in place of the native Asp30-Ala31 sequence of Rop. They also all contained a C-terminal 6×His purification tag. The full sequences of the Rop translation products are provided below.

RopN
SEQ ID NO: 21
MDYKDDDDKMTKQEKTALNMARFIRSQTLTLLEKLNELMCAYDGGGGGD
EQADICESLHDHADELYRSCLARFGDDGENLHHHHHH
RopM
SEQ ID NO: 22
MDYKDDDDKMTKQEKTALNMARFIRSQTLTLLEKLNELGGMCAYDGGGD
EQADICESLHDHADELYRSCLARFGDDGENLHHHHHH
RopC
SEQ ID NO: 23
MDYKDDDDKMTKQEKTALNMARFIRSQTLTLLEKLNELGGGGMCAYDGD
EQADICESLHDHADELYRSCLARFGDDGENLHHHHHH
RopC
SEQ ID NO: 24
MDYKDDDDKMTKQEKTALNMARFIRSQTLTLLEKLNELGGGGMAAYDGD
EQADICESLHDHADELYRSCLARFGDDGENLHHHHHH
RopCG4
SEQ ID NO: 25
MDYKDDDDKMTKQEKTALNMARFIRSQTLTLLEKLNELGGGGMCGGGGD
EQADICESLHDHADELYRSCLARFGDDGENLHHHHHH

Cloning Rop Constructs into pET-32(a)+ Vector

The sequences encoding RopN, RopM, RopC, RopC−, and RopCG4 were cloned into a pET-32a(+) plasmid as follows. Circular pET-32a(+) vector (1 μg, Millipore Sigma, catalog #69015-3) was incubated with 1 L each of restrictions enzymes NdeI (New England Biosciences, catalog #R0111S) and NotI-HF (New England Biosciences, catalog #R3189S) in cutSmart Buffer (New England Biolabs, catalog #B7204S) at 37° C. for 1 hour. The entire restriction digest reaction was run on a 0.8% agarose gel at 150V for 45 minutes and 5.4 kbp and 0.5 kbp fragments were observed using a blue light transilluminator. The larger 5.4 kbp fragment was excised from the gel and purified using a Monarch DNA Gel Extraction Kit (NEB, catalog #T1020S). Concentration of linearized pET-32(a)+ vector was determined by absorbance at 260 nm. 33.3 ng of purified, linearized pET-32(a)+ vector and 100 ng of respective gBlock DNA fragment (Integrated DNA Technologies, Coralville, IA) encoding Rop constructs were combined in a 10 μL Gibson Assembly reaction containing HiFI DNA Assembly Master Mix (NEB, catalog #E2621L) and incubated at 50° C. for 1 hour to generate circular pET-32(a)+ vectors containing coding sequences for Rop constructs. Circularized plasmids from the previous step were transformed into NEB 5-alpha competent E. coli (NEB, catalog #C2987H). First, cells were thawed on ice for 10 minutes after which 4 μL of the previous Gibson Assembly reaction was added to cells and placed back on ice for 30 minutes. After addition of plasmid, cells were subjected to heat shock at 42° C. for 30 seconds and placed on ice for 5 minutes. 900 L of SOC outgrowth medium (NEB, catalog #B9020S) was added to cells and cells were incubated at 37° C. for 1 hour with shaking at 200 rpm. Agar plates containing 100 μg/mL carbenicillin were inoculated with 100 L of transformed cells and grown overnight at 37° C. 5 single colonies per construct were picked and inoculated into 5 mL LB+100 μg/mL carbenicillin and grown for 16 hours at 37° C. Pure plasmid was isolated from 5 mL cultures using Qiaprep Spin Miniprep Kit (Qiagen, catalog #27106) and sequences were confirmed by Sanger sequencing at the UC Berkeley DNA Sequencing Facility. Plasmids containing the precise coding sequence for each construct were transformed into chemically competent BL21 E. coli (NEB, catalog #C2530H) for large scale protein expression following the same transformation protocol detailed above.

RopN gBlock Sequence
SEQ ID NO: 26
TCCCCTCTAGAAATAATTTTGTTTAACTTTAAGAAGGAGATATACATATGGA
CTACAAAGACGACGACGACAAAATGACCAAACAGGAAAAAACCGCCCTTAACATG
GCCCGCTTTATCAGAAGCCAGACATTAACGCTTCTGGAGAAACTCAACGAGCTGAT
GTGCGCCTACGACGGAGGGGGCGGTGGCGATGAACAGGCAGACATCTGTGAATCG
CTTCACGACCACGCTGATGAGCTTTACCGCAGCTGCCTCGCGCGTTTCGGTGATGAC
GGTGAAAACCTCCACCACCATCACCATCATTAAGCGGCCGCACTCGAGCACC
RopM gBlock Sequence
SEQ ID NO: 27
TCCCCTCTAGAAATAATTTTGTTTAACTTTAAGAAGGAGATATACATATGGA
CTACAAAGACGACGACGACAAAATGACCAAACAGGAAAAAACCGCCCTTAACATG
GCCCGCTTTATCAGAAGCCAGACATTAACGCTTCTGGAGAAACTCAACGAGCTGGG
CGGGATGTGCGCCTACGACGGAGGTGGCGATGAACAGGCAGACATCTGTGAATCGC
TTCACGACCACGCTGATGAGCTTTACCGCAGCTGCCTCGCGCGTTTCGGTGATGACG
GTGAAAACCTCCACCACCATCACCATCATTAAGCGGCCGCACTCGAGCACC
RopC gBlock Sequence
SEQ ID NO: 28
TCCCCTCTAGAAATAATTTTGTTTAACTTTAAGAAGGAGATATACATATGGA
CTACAAAGACGACGACGACAAAATGACCAAACAGGAAAAAACCGCCCTTAACATG
GCCCGCTTTATCAGAAGCCAGACATTAACGCTTCTGGAGAAACTCAACGAGCTGGG
CGGGGGGGGAATGTGCGCCTACGACGGAGATGAACAGGCAGACATCTGTGAATCG
CTTCACGACCACGCTGATGAGCTTTACCGCAGCTGCCTCGCGCGTTTCGGTGATGAC
GGTGAAAACCTCCACCACCATCACCATCATTAAGCGGCCGCACTCGAGCACC
RopC− gBlock Sequence
SEQ ID NO: 29
TCCCCTCTAGAAATAATTTTGTTTAACTTTAAGAAGGAGATATACATATGGACTACA
AAGACGACGACGACAAAATGACCAAACAGGAAAAAACCGCCCTTAACATGGCCCG
CTTTATCAGAAGCCAGACATTAACGCTTCTGGAGAAACTCAACGAGCTGGGCGGAG
GTGGAATGGCCGCCTACGACGGAGATGAACAGGCAGACATCTGTGAATCGCTTCAC
GACCACGCTGATGAGCTTTACCGCAGCTGCCTCGCGCGTTTCGGTGATGACGGTGA
AAACCTCCACCACCATCACCATCATTAAGCGGCCGCACTCGAGCACC
RopCG4 gBlock Sequence
SEQ ID NO: 30
TCCCCTCTAGAAATAATTTTGTTTAACTTTAAGAAGGAGATATACATATGGACTACA
AAGACGACGACGACAAAATGACCAAACAGGAAAAAACCGCCCTTAACATGGCCCG
CTTTATCAGAAGCCAGACATTAACGCTTCTGGAGAAACTCAACGAGCTGGGCGGGG
GTGGAATGTGCGGTGGGGGAGGAGATGAACAGGCAGACATCTGTGAATCGCTTCAC
GACCACGCTGATGAGCTTTACCGCAGCTGCCTCGCGCGTTTCGGTGATGACGGTGA
AAACCTCCACCACCATCACCATCATTAAGCGGCCGCACTCGAGCACC

Expression and Purification of mCherry Variants

Starter cultures of 2 mL of Miller's LB Broth (AmericanBio, catalog #AB01201) supplemented with 100 μg/mL carbenicillin were inoculated with a single colony of E. coli BL21 (DE3) harboring the plasmid of interest and grown overnight at 37° C. with shaking at 200 rpm. The starter culture (2 mL) was used to inoculate a 200 mL expression culture of Miller's LB Broth also supplemented with 100 μg/mL carbenicillin. The expression culture was grown at 37° C. with shaking at 200 rpm to an OD₆₀₀ of 0.6 at which point it was induced with 1 mM IPTG, transferred to a 20° C. incubator, and grown for 24 hours with shaking at 200 rpm. The expression culture was harvested by centrifugation at 4,300×g at 4° C. for 45 minutes. The resulting cell pellet was suspended in 10 mL of Lysis Buffer (20 mM Tris-HCl, 500 mM NaCl, pH 8.0) containing 1 tablet of cOmplete, mini EDTA-free ULTRA protease inhibitor cocktail (Sigma-Aldrich, St. Louis, MO). The cell suspension was disrupted by sonication on ice (Branson Sonifier 250, 3 cycles of 30 second pulse at 30% duty cycle and microtip limit of 5 followed by 60 second pause). The cell lysate was cleared by centrifugation at 23,000×g at 4° C. for 20 minutes. TALON® Metal Affinity Resin (2 mL) (Takara Biosciences, catalog #635504) was equilibrated with Lysis Buffer, added to the cleared cell lysate, and incubated on a rotisserie at 4° C. for 1 hour. The TALON® resin-lysate mixture was then passed through a gravity flow Poly-Prep Chromatography Column (Bio-Rad Laboratories, Hercules, CA). Non-specifically bound proteins were removed by washing the Ni-NTA column with 10 mL of Wash Buffer (20 mM Tris-HCl, 500 mM NaCl, 20 mM imidazole pH 8.0). The 6×His-tagged protein was eluted by washing the Ni-NTA column with 5 mL of Elution Buffer (20 mM Tris-HCl, 500 mM NaCl, 250 mM imidazole pH 8.0). The purified protein was loaded into a β-12 mL Slide-A-Lyzer Dialysis Cassette with a 10 kDa molecular weight cut off (Thermo Scientific, Waltham, MA) and dialyzed against 1 L of Storage Buffer (20 mM HEPES, 150 mM NaCl, 1 mM TCEP, pH 7.4) at 4° C. for at least 18 hours. The dialyzed protein was concentrated using Amicon Ultra-15 mL centrifugal filters with a 10 kDa molecular weight cut off (MilliporeSigma, Burlington, MA) according to the manufacturer's instructions. The purified protein was quantified using absorbance at 280 nm, diluted to 1 mM using Storage Buffer, snap frozen as single-use aliquots, and stored at −80° C. The typical expression yield of mCherry constructs using the above protocol was 90 mg/L of E. coli culture.

### Expression and Purification of Rop Constructs

Starter cultures of 2 mL of Miller's LB Broth (AmericanBio, catalog #AB01201) supplemented with 100 μg/mL carbenicillin were inoculated with a single colony of E. coli BL21 (DE3) harboring the plasmid of interest and grown overnight at 37° C. with shaking at 200 rpm. The starter culture (2 mL) was used to inoculate a 200 mL expression culture of Miller's LB Broth also supplemented with 100 μg/mL carbenicillin. The expression culture was grown at 37° C. with shaking at 200 rpm to an OD₆₀₀ of 0.6 at which point it was induced with 1 mM IPTG, transferred to a 20° C. incubator, and grown for 24 hours with shaking at 200 rpm. The expression culture was harvested by centrifugation at 4,300×g at 4° C. for 45 minutes. The resulting cell pellet was suspended in 10 mL of Lysis Buffer (20 mM Tris-HCl, 500 mM NaCl, pH 8.0) containing 1 tablet of cOmplete, mini EDTA-free ULTRA protease inhibitor cocktail (Sigma-Aldrich, St. Louis, MO). The cell suspension was disrupted by sonication on ice (Branson Sonifier 250, 3 cycles of 30 second pulse at 30% duty cycle and microtip limit of 5 followed by 60 second pause). The cell lysate was cleared by centrifugation at 23,000×g at 4° C. for 20 minutes. TALON® Metal Affinity Resin (2 mL) (Takara Biosciences, catalog #635504) was equilibrated with lysis buffer (20 mM Tris-HCl, 500 mM NaCl, pH 8.0). The equilibrated Ni-NTA agarose (2 mL) was added to the cleared cell lysate and incubated on a rotisserie at 4° C. for 1 hours. The TALON® resin-lysate mixture was then passed through a gravity flow Poly-Prep Chromatography Column (Bio-Rad Laboratories, Hercules, CA). Non-specifically bound proteins were removed by washing the TALON® column with 10 mL of wash buffer (20 mM Tris-HCl, 500 mM NaCl, 20 mM imidazole pH 8.0). The 6×His-tagged protein was eluted by washing the TALON® column with 5 mL of elution buffer (20 mM Tris-HCl, 500 mM NaCl, 250 mM imidazole pH 8.0). The purified protein was loaded into a β-12 mL Slide-A-Lyzer Dialysis Cassette with a 3.5 kDa molecular weight cut off (Thermo Scientific, Waltham, MA) and dialyzed against 1 L of storage buffer (20 mM HEPES, 150 mM NaCl, 1 mM TCEP, pH 7.4) at 4° C. for at least 18 hours. The dialyzed protein was concentrated using Amicon Ultra-15 mL centrifugal filters with a 3 kDa molecular weight cut off (MilliporeSigma, Burlington, MA) according to the manufacturer's instructions. The purified protein was quantified using absorbance at 280 nm, diluted to 1 mM using storage buffer (20 mM HEPES, 150 mM NaCl, 1 mM TCEP, pH 7.4), snap frozen as single use aliquots, and stored at −80° C. The typical expression yield of Rop construct using the above protocol was 40 mg/L of E. coli culture.

Characterization of mCherry and Rop Constructs

SDS-PAGE. Purified proteins (4 μg) were mixed (4:1) with SDS-PAGE sample buffer (5% β-Mercaptoethanol, 0.02% bromophenol blue, 30% glycerol, 10% SDS, 250 mM Tris-HCl, pH 6.8) and incubated at 95° C. for 5 minutes before being applied to a 4-15% mini-PROTEAN TGX precast gel (Bio-Rad Laboratories, Hercules, CA) run at 200 V for 30 minutes in Tris-Glycine-SDS running buffer (3 g/L tris, 14.4 g/L glycine, 1 g/L sodium dodecyl sulfate, pH 8.3) alongside a lane containing Precision Plus Protein Dual Color Standards (Bio-Rad Laboratories, Hercules, CA). Protein bands were visualized using Coomassie stain (1 g/L Coomassie Brilliant Blue in methanol:water:acetic acid (5:4:1)) and imaged using a Bio-Rad Chemidoc MP Imaging System. Band intensities were quantified using the gel analysis tool of FIJI.5

### LC-MS Analysis of Purified Proteins

LC-MS analysis was performed on an Agilent 1290 Infinity II HPLC connected to an Agilent 6530B QTOF AJS-ESI. The mobile phase for LC-MS was water and acetonitrile with 0.1% (v/v) formic acid and a flow rate of 0.4 mL/min. Each sample was injected onto a Poroshell 300SB-C8 column (2.1×75 mm, 5-Micron, room temperature, Agilent) using a linear gradient from 5 to 95% acetonitrile over 9 minutes after an initial hold at 5% acetonitrile for 0.5 minutes (0.4 mL/min). The following parameters were used during acquisition: Fragmentor voltage 225 V, gas temperature 300° C., gas flow 10 L/min, sheath gas temperature 350° C., sheath gas flow 11 L/min, nebulizer pressure 35 psi, skimmer voltage 65 V, Vcap 5000 V, 1 spectra/s. Intact protein masses were obtained via deconvolution using the Maximum Entropy algorithm in Mass Hunter Bioconfirm (V10, Agilent).

Reactions with MicD-F and/or ArtGox
Reaction of Peptides and Proteins with MicD-F

A typical reaction scale for analysis via LC-MS was 30 μL. Peptide substrate stock (3 μL) suspended at 1 mM (10 mM bicine, 150 mM NaCl, 1 mM TCEP, pH 9.0) was added to 24 μL of reaction buffer (6.25 mM ATP, 6.25 mM MgCl₂, 100 mM bicine, 150 mM NaCl, 1 mM TCEP, pH 8.0 or 9.0). Separately, an aliquot of MicD-F (500 M in 20 mM HEPES, 150 mM NaCl, 1 mM TCEP, pH 7.4) was thawed on ice. The thawed enzyme aliquot was then diluted with cold storage buffer (20 mM HEPES, 150 mM NaCl, 1 mM TCEP, pH 7.4) to a concentration equal to 10× the desired enzyme concentration. The peptide and enzyme solutions were then incubated at 37° C. for 15 minutes. After temperature equilibration, enzyme solution (3 μL) was added to the peptide solution and gently pipette mixed. The resulting solution was then incubated at 37° C. for the indicated amount of time before LC-MS analysis.

Reactions of proteins with MicD-F were performed in the manner described above with minor modifications. Namely, the substrate stock was a 1 mM solution of protein in 20 mM HEPES, 150 mM NaCl, 1 mM TCEP, pH 7.4.

Reaction of Peptides and Proteins with MicD-F and ArtGox in Tandem

A typical reaction scale for analysis via LC-MS was 30 μL. Peptide substrate stock (3 L) suspended at 1 mM in 10 mM bicine, 150 mM NaCl, 1 mM TCEP, pH 9.0 was added to 21 L of reaction buffer (7.14 mM ATP, 7.14 mM MgCl₂, 2.86 mM FMN, 100 mM bicine, 150 mM NaCl, 1 mM TCEP, pH 8.0 or 9.0). Separately, aliquots of MicD-F (500 M in 20 mM HEPES, 150 mM NaCl, 1 mM TCEP, pH 7.4) and ArtGox (800 M in 20 mM HEPES, 150 mM NaCl, 1 mM TCEP, pH 7.4) were thawed on ice. The thawed enzyme aliquots were then diluted with cold storage buffer (20 mM HEPES, 150 mM NaCl, 1 mM TCEP, pH 7.4) to a concentration equal to 10× the desired enzyme concentration. The peptide and enzyme solutions were then incubated at 37° C. for 15 minutes. After temperature equilibration, enzyme solutions (3 μL of each) were added to the peptide solution and gently pipette mixed. The resulting solution was then incubated at 37° C. for the indicated amount of time before LC-MS analysis.

Reactions of proteins with MicD-F and ArtGox were performed in the manner described above with minor modifications. The substrate stock was a 1 mM solution of protein in 20 mM HEPES, 150 mM NaCl, 1 mM TCEP, pH 7.4. Reactions were carried out at either 25° C. or 37° C. as indicated.

LC-MS Analysis of Reactions with Peptide Substrates

To remove 6×His-tagged enzymes, Ni-NTA (Qiagen, Hilden, Germany) slurry and crude reaction were mixed 1:1 by volume and incubated on ice for 30 minutes with occasional agitation. The Ni-NTA resin was then removed by centrifugation at 21,300×g for 10 minutes at 4° C. Enzyme-depleted reaction mixture (1 μL) was used for LC-MS analysis which was performed on an Agilent 1290 Infinity II HPLC connected to an Agilent 6530B QTOF AJS-ESI. The mobile phase for LC-MS was water and acetonitrile with 0.1% (v/v) formic acid and a flow rate of 0.7 mL/min. Each sample was injected onto an Eclipse XDB C-18 column (2.1×50 mm, 1.8-Micron, room temperature, Agilent) and separated using a linear gradient from 5 to 95% acetonitrile over 4.5 minutes after an initial hold at 5% acetonitrile for 0.5 minutes. The following parameters were used during acquisition: Fragmentor voltage 175 V, gas temperature 300° C., gas flow 8 L/min, sheath gas temperature 350° C., sheath gas flow 11 L/min, nebulizer pressure 35 psi, skimmer voltage 65 V, Vcap 3500 V, 1 spectra/s.

LC-MS Analysis of Reactions with Protein Substrates

Unprocessed reaction mixture (1 μL) was used for LC-MS analysis which was performed on an Agilent 1290 Infinity II HPLC connected to an Agilent 6530B QTOF AJS-ESI. The mobile phase for LC-MS was water and acetonitrile with 0.1% (v/v) formic acid and a flow rate of 0.4 mL/min. Each sample was injected onto a Poroshell 300SB-C8 column (2.1×75 mm, 5-Micron, room temperature, Agilent) using a linear gradient from 5 to 55% acetonitrile over 8 minutes after an initial hold at 5% acetonitrile for 2 minutes (0.4 mL/min). The following parameters were used during acquisition: Fragmentor voltage 225 V, gas temperature 300° C., gas flow 10 L/min, sheath gas temperature 350° C., sheath gas flow 11 L/min, nebulizer pressure 35 psi, skimmer voltage 65 V, Vcap 5000 V, 1 spectra/s. Intact protein masses were obtained via deconvolution using the Maximum Entropy algorithm in Mass Hunter Bioconfirm (V10, Agilent).

### Characterization of Rop Variants

### Purification of RopC-U and RopC-Z Via Size-Exclusion Chromatography

Reactions to synthesize and purify thiazoline (RopC-U)- and thiazole (RopC-Z)-modified Rop variants were carried out in a total reaction volume of 1.5 mL. All stock solutions and reaction components were scaled up in accordance with the analytical scale reaction protocols described above. To synthesize RopC-U, RopC (100 μM) was reacted with MicD-F (50 μM) at pH 9.0 and 37° C. To synthesize RopC-Z, RopC (100 μM) was reacted with MicD-F (50 μM) and ArtGox (80 μM) at pH 9.0 and 37° C. Reaction progress was monitored in the manner described above for LC-MS analysis of analytical scale protein reactions. Once the reaction was complete as judged by LC-MS, RopC variants were separated from the crude reaction mixture via size exclusion chromatography (SEC).

A HiLoad® 16/600 Superdex® 75 pg column (stored and operated at 4° C.) was washed with 2 column volumes (CV) of degassed and sterile filtered MilliQ water. The column was then equilibrated in running buffer (10 mM phosphate, 100 mM NaCl, 150 M TCEP, pH 7.0) for 2 CV. The crude reaction mixture (1.5 mL) was applied to a 5 mL sample loop. The sample loop was washed with 10 mL of running buffer at 1 mL/min to load the sample onto the column. The sample was then eluted from the column by flowing running buffer at 1 mL/min for 1.5 CV. Fractions were collected in 1 mL aliquots for the entirety of sample application and elution. Fractions were analyzed via SDS-PAGE analysis and those containing protein of the correct molecular weight (approximately 10 kDa) were pooled and concentrated to 20 M using Amicon Ultra-0.5 mL centrifugal filters with a 3 kDa molecular weight cut off (MilliporeSigma, Burlington, MA) according to the manufacturer's instructions. The 20 M protein solution was equally divided into 3 parts and flash frozen on liquid nitrogen before analysis via circular dichroism.

### Characterization of Rop Variants Via Analytical Size-Exclusion Chromatography

To assess the homogeneity of isolated Rop variants, a solution of each protein was prepared (250 μg at 50 μM) in running buffer (10 mM phosphate, 100 mM NaCl, 150 M TCEP, pH 7.0). A Superdex® 75 Increase 10/300 GL column (stored and operated at 4° C.) was washed with 2 column volumes (CV) of degassed and sterile filtered MilliQ water. The column was then equilibrated in running buffer for 2 CV. Each sample (500 μL) was applied to a 500 μL sample loop. The sample loop was washed with 2 mL of running buffer at 0.8 mL/min to load the sample onto the column. The sample was then eluted from the column by flowing running buffer at 0.8 mL/min for 1.30 CV. To assess column performance, a gel filtration standard (Bio-Rad Laboratories, Hercules, CA, catalog number 151-1901) containing 670 kDa, 158 kDa, 44 kDa, 17 kDa, and 1.35 kDa standards was used according to the manufacturer's instructions.

### Characterization of Rop Variants Via Circular Dichroism (CD)

RopN, RopM, and RopC were exchanged into CD buffer (10 mM phosphate, 100 mM NaCl, 150 μM TCEP, pH 7.0) using Amicon Ultra-0.5 mL centrifugal filters (MilliporeSigma, Burlington, MA) with a 3 kDa molecular weight cut-off according to the manufacturer's instructions and then diluted with the same buffer to a concentration of 20 μM. For CD analysis each Rop variant was transferred to a 1 mm quartz cuvette. Wavelength and temperature dependent CD spectra were collected with an AVIV Biomedical, Inc. (Lakewood, NJ) Circular Dichroism Spectrometer Model 410. For wavelength-dependent spectra, initial scans were performed from 200 to 300 nm at 25° C. in 2 nm increments with an averaging time of 5 seconds. For temperature melt experiments, the signal was monitored at 222 nm with an averaging time of 5 seconds. Temperature melts were performed from 5 to 90° C. in 1° C. increments with equilibration for 2 minutes before each measurement. Following the temperature melt the sample was returned to 25° C. and the wavelength-dependent CD spectra was measured once more to assess the reversibility of the melt. Raw data (mdeg) were converted to molar ellipticity ([Θ], in deg*cm²*dmol⁻¹) by

$$[ Θ ] = ( mdeg * M ) / ( 10 * L * C ) \qquad Equation 1$$

where M is the mean residual weight (116.00 g/mol for RopN, RopM, and RopC), L is the pathlength of the cuvette in centimeters, and C is the concentration of the sample in g/L.7 The melting temperature was determined by fitting the molar ellipticity as a function of temperature to a Boltzmann sigmoidal curve using GraphPad Prism (Version 7.04).

CD analysis of RopC-U and RopC-Z was performed as described for RopN, RopM, and RopC with the following modifications. Temperature melts were performed from 5 to 90° C. in 2.5° C. increments. Before measuring the temperature melt for RopC-Z, the protein was refolded by performing an initial melt from 5 to 90° C. in 2.5° C. increments as described.

TABLE 1
Sequences, predicted, and observed masses of peptide substrates described in
this work. Notes: All synthetic peptides were prepared as C-terminal carboxylic
acids with a free N-terminus. Predicted and observed masses are reported for
the [M + H]⁺ ion of each peptide.

| | | Predicted | Observed |
|---|---|---|---|
| Peptide | Peptide Sequence | Mass (Da) | Mass (Da) |
| +1 site model | SEQ ID NO: 31 | 641.2599 | 641.2671 |
| substrate | ICAYDG | | |
| 1a | SEQ ID NO: 32 | 632.2021 | 632.2060 |
| | [Bz]-CAYDG | | |
| 1b | SEQ ID NO: 33 | 704.1644 | 704.1688 |
| | [TFBz]-CAYDG | | |
| 1c | SEQ ID NO: 34 | 700.1919 | 700.1954 |
| | [Coumarin]-CAYDG | | |
| 1d | SEQ ID NO: 35 | 647.2130 | 647.2173 |
| | [2-Abz]-CAYDG | | |
| 1e | SEQ ID NO: 36 | 677.2236 | 677.2293 |
| | [5-Methoxy-2-Abz]-CAYDG | | |
| 1f | SEQ ID NO: 37 | 648.2082 | 648.2134 |
| | [Apy]-CAYDG | | |
| 1g | SEQ ID NO: 38 | 655.2756 | 655.2819 |
| | [B3-HIle]-CAYDG | | |
| 1h | SEQ ID NO: 39 | 653.2599 | 653.2667 |
| | [ACHC]-CAYDG | | |
| 1i | SEQ ID NO: 40 | 639.2443 | 639.2480 |
| | [ACPC]-CAYDG | | |
| +1 site model | SEQ ID NO: 41 | 754.3440 | 754.3555 |
| substrate | ICIAYDG | | |
| 4a | SEQ ID NO: 42 | 768.3597 | 768.3724 |
| | IC-[B3-HIle]-AYDG | | |
| 4b | SEQ ID NO: 43 | 740.3284 | 740.3353 |
| | IC-[B2-Dimethyl]-AYDG | | |
| 4c | SEQ ID NO: 44 | 766.3440 | 766.3537 |
| | IC-[ACHC]-AYDG | | |
| 4d | SEQ ID NO: 45 | 752.3284 | 752.3361 |
| | IC-[ACPC]-AYDG | | |
| 4e | SEQ ID NO: 46 | 760.2971 | 760.3067 |
| | IC-[2-Abz]-AYDG | | |
| 4f | SEQ ID NO: 47 | 790.3076 | 790.3158 |
| | IC-[5-Methoxy-2-Abz]-AYDG | | |
| 4g | SEQ ID NO: 48 | 761.2923 | 761.2977 |
| | IC-[Apy]-AYDG | | |

TABLE 2
mCherry variants.

| | | Insertion | Parent MW | Mature MW |
|---|---|---|---|---|
| ID | Insertion | Site | (Da) | (Da) |
| mCherryC+ | SEQ ID NO: 49 | C-terminus | 30164 | 30142 |
| | -MCAYDG- | | | |
| mCherryC- | SEQ ID NO: 50 | C-terminus | 30132 | 30110 |
| | -MAAYDG- | | | |
| mCherry 137+ | SEQ ID NO: 51 | D137 | 29312 | 29290 |
| | -MCAYDG- | | | |
| mCherry192+ | SEQ ID NO: 52 | V192 | 29312 | 29290 |
| | -MCAYDG- | | | |
| mCherry 174+ | SEQ ID NO: 53 | D174 | 29312 | 29290 |
| | -MCAYDG- | | | |
| mCherry211+ | SEQ ID NO: 54 | E211 | 29312 | 29290 |
| | -MCAYDG- | | | |

Table 3
Rop variants.

| ID | Loop Sequence | MW (Da) |
|---|---|---|
| RopN | SEQ ID NO: 55 | 9859.81 |
| | MCAYDGGGGG | |
| RopM | SEQ ID NO: 56 | 9859.81 |
| | GGMCAYDGGG | |
| RopC | SEQ ID NO: 57 | 9859.81 |
| | GGGGMCAYDG | |
| RopCG4 | SEQ ID NO: 58 | 9681.62 |
| | GGGGMCGGGG | |
| RopC− | SEQ ID NO: 59 | 9827.75 |
| | GGGGMAAYDG | |

### SUPPLEMENTARY REFERENCES

(1) Lee, H. B.; Zaccaro, M. C.; Pattarawarapan, M.; Roy, S.; Saragovi, H. U.; Burgess, K. Syntheses and Activities of New C10β-Turn Peptidomimetics. J. Org. Chem. 2004, 69(3), 701-713. https://doi.org/10.1021/jo034167x.
(2) Ge, Y.; Czekster, C. M.; Miller, O. K.; Botting, C. H.; Schwarz-Linek, U.; Naismith, J. H. Insights into the Mechanism of the Cyanobactin Heterocyclase Enzyme. Biochemistry 2019, 58(16), 2125-2132. https://doi.org/10.1021/acs.biochem.9b00084.
(3) Gao, S.; Ge, Y.; Bent, A. F.; Schwarz-Linek, U.; Naismith, J. H. Oxidation of the Cyanobactin Precursor Peptide Is Independent of the Leader Peptide and Operates in a Defined Order. Biochemistry 2018, 57(41), 5996-6002. https://doi.org/10.1021/acs.biochem.8b00835.
(4) Liu, H.; Naismith, J. H. A Simple and Efficient Expression and Purification System Using Two Newly Constructed Vectors. Protein Expression and Purification 2009, 63(2), 102-111. https://doi.org/10.1016/j.pep.2008.09.008.
(5) Schindelin, J.; Arganda-Carreras, I.; Frise, E.; Kaynig, V.; Longair, M.; Pietzsch, T.; Preibisch, S.; Rueden, C.; Saalfeld, S.; Schmid, B.; Tinevez, J.-Y.; White, D. J.; Hartenstein, V.; Eliceiri, K.; Tomancak, P.; Cardona, A. Fiji: An Open-Source Platform for Biological-Image Analysis. Nat Methods 2012, 9(7), 676-682. https://doi.org/10.1038/nmeth.2019.
(6) Gibson, D. G.; Young, L.; Chuang, R.-Y.; Venter, J. C.; Hutchison, C. A.; Smith, H. O. Enzymatic Assembly of DNA Molecules up to Several Hundred Kilobases. Nature Methods 2009, 6(5), 343-345. https://doi.org/10.1038/nmeth.1318.
(7) Greenfield, N. J. Using Circular Dichroism Spectra to Estimate Protein Secondary Structure. Nat Protoc 2006, 1(6), 2876-2890. https://doi.org/10.1038/nprot.2006.202.
(8) Hirel, P. H.; Schmitter, M. J.; Dessen, P.; Fayat, G.; Blanquet, S. Extent of N-Terminal Methionine Excision from Escherichia coli Proteins Is Governed by the Side-Chain Length of the Penultimate Amino Acid. Proceedings of the National Academy of Sciences 1989, 86(21), 8247-8251. https://doi.org/10.1073/pnas.86.21.8247.
(9) Xiao, Q.; Zhang, F.; Nacev, B. A.; Liu, J. O.; Pei, D. Protein N-Terminal Processing: Substrate Specificity of Escherichia coli and Human Methionine Aminopeptidases. Biochemistry 2010, 49(26), 5588-5599. https://doi.org/10.1021/bi1005464.
(10) Gross, L. A.; Baird, G. S.; Hoffman, R. C.; Baldridge, K. K.; Tsien, R. Y. The Structure of the Chromophore within DsRed, a Red Fluorescent Protein from Coral. Proceedings of the National Academy of Sciences 2000, 97(22), 11990-11995. https://doi.org/10.1073/pnas.97.22.11990.
(11) Feiner, R. C.; Penne, I.; Müller, B.; Müller, K. M. EGF-MCherry Fusion Protein Expressed in E. coli Shows Product Heterogeneity but a High Biological Activity. Biochemistry 2019, 58(8), 1043-1047. https://doi.org/10.1021/acs.biochem.9b00021.

1. A method of polymer engineering comprising: deploying a constitutively active dehydratase enzyme of RiPP biosynthesis to accept a substrate containing a non-α-amino acid monomer at a position flanking the reaction site.

2. The method of claim 1, wherein the polymer comprises an amino acid polymer compatible and operable with the disclosed constitutively active dehydratase enzyme of RiPP biosynthesis to accept a substrate containing a non-α-amino acid monomer at a position flanking the reaction site.

3. The method of claim 1, wherein the polymer comprises an antibody, cytokine, replacement enzyme, or therapeutic protein.

4. The method of claim 1, wherein the method imbues a polymeric material comprising the polymer with new structures or functions.

5. The method of claim 1, wherein the polymer comprises a protein, and the method introduces a non-native backbone modification to expand protein function.

6. The method of claim 1, used to engineer a therapeutic protein to express at higher levels, resist degradation, improve thermal and proteolytic stability, alter immunogenicity, antigenicity or immune reactivity, or encode a function, such as targeting the protein to distinct cells or tissues.

7. The method of claim 1, wherein the enzyme catalyzes formation of an azole ring.

8. The method of claim 1, wherein the enzyme catalyzes formation of an oxazole or thiazole ring.

9. The method of claim 1, wherein the enzyme is a constitutively active heterocyclase in which the leader sequence is fused directly to the enzyme, such as LynD fusion (LynD-F) and MicD fusion (MicD-F).

10. The method of claim 1, wherein the enzyme is selected from MicD-F and ArtGox.

11. The method of claim 1, wherein the enzyme is selected from heterocyclases MicD, PatD, and LynD.

12. The method of claim 1, providing a protein therapeutic fused to a RiPP natural product, synthesized in a single step, without a separate chemical bio-conjugation step, without sequential or separate purification steps, and in situ.

13. The method of claim 1, wherein:
(a) multiple, structurally diverse aromatic rings are tolerated at the +1 position that precedes the site of cyclization;
(b) multiple, structurally diverse beta-β-amino acids are tolerated at the +1 site; or
(c) aramid monomers are tolerated at the −1 site.

14. The method of claim 1, wherein
(a) benzoic acid, tetrafluoro-benzoic acid, 2-amino benzoic acid, 2-amino-5-methoxy benzoic acid, 2-aminopyridyl, and coumarin are tolerated at the +1 position that precedes the site of cyclization; or
(b) beta-β-isoleucine is tolerated at the +1 site.

15. The method of claim 1, wherein the enzyme processes a substrate comprising:
(a) an aromatic ring, even at the +1 position that precedes the site of cyclization;
(b) beta-β-amino acid, even at the +1 site; or
(c) an aramid monomer, even at the −1 site.

16. The method of claim 1, wherein the enzyme processes a substrate comprising:
(a) benzoic acid, tetrafluoro-benzoic acid, 2-amino benzoic acid, 2-amino-5-methoxy benzoic acid, 2-aminopyridyl, or coumarin, even at the +1 position that precedes the site of cyclization; or
(b) beta-β-isoleucine, even at the +1 site.

17. The method of claim 1, wherein the method installs heterocyclic backbones within protein loops and linkers without disrupting the native tertiary fold.

18. The method of claim 1, wherein the enzyme introduces an azoline heterocycle into one or more loops of a target protein to generate proteins with altered and improved properties.

19. The method of claim 1, wherein the enzyme that is MicD introduces an azoline heterocycle into one or more loops of a target protein that is GFP to generate proteins with altered and improved properties.


SEQUENCE LISTING (59 sequences)

SEQ ID NO: 1 - protein, 86 aa, Escherichia coli
MDYKDDDDKMTKQEKTALNMARFIRSQTLTLLEKLNELMCAYDGGGGGDEQADICESLHDHADELYRSCLARFGDDGENLHHHHHH

SEQ ID NO: 2 - protein, 86 aa, Escherichia coli
MDYKDDDDKMTKQEKTALNMARFIRSQTLTLLEKLNELGGMCAYDGGGDEQADICESLHDHADELYRSCLARFGDDGENLHHHHHH

SEQ ID NO: 3 - protein, 86 aa, Escherichia coli
MDYKDDDDKMTKQEKTALNMARFIRSQTLTLLEKLNELGGGGMCAYDGDEQADICESLHDHADELYRSCLARFGDDGENLHHHHHH

SEQ ID NO: 4 - protein, 86 aa, Escherichia coli
MDYKDDDDKMTKQEKTALNMARFIRSQTLTLLEKLNELGGGGMCGGGGDEQADICESLHDHADELYRSCLARFGDDGENLHHHHHH

SEQ ID NO: 5 - protein, 62 aa, Escherichia coli
MDYKDDDDKMTKQEKTALNMARFIRSQTLTLLEKLNELGGGGMAAYDGDEQADICESLHDHA

SEQ ID NO: 6 - protein, 24 aa, Escherichia coli
DELYRSCLARFGDDGENLHHHHHH

SEQ ID NO: 7 - protein, 829 aa, Microcystis aeruginosa
MSHHHHHHDYDENLYFQGSRLTAGQLSSQLAELSEEALGDAGAGAGAGAGAKLMQSTPLLQIQPHFHVEVIEPKQVYLLGEQANYALTGQLYCQILPLLDGQHSREQIVEKLDGEVPSEYIDYVLDRLAEKGYLTEAAPELSSEVAAFWSELGIAPPVAAEALRQSVTLTPVGNISEVTVAALTTALRDIGISVQTPTEAGSPTALNVVLTDDYLQPELAKINKQALESQQTWLLVKPVGSVLWLGPVFVPGKTGCWDCLAHRLRGNREVEASVLQQKQAQQQRNGQSGSVIGCLPTARATLPSTLQTGLQFAATEIAKWIVKHHVKATAPGTVFFPTLDGKIITFNHTVIDLKSHVLVRRSQCPSCGDRQILHRQGFEPVKLVSRRKHFTHDGGHRAFTPEQTVQKYQHLVSPITGVVTELVRLTDPANPLVHTYKAGHAFGSATTLRGLRNTLKYKSSGKGKTDIQSRASGLCEAIERYSGIFQGDEPRKRATLAELGDLALHPESLLYFSNTQYANREELNAQGSAAAYRWIPNRFDVSQAIDWTPVWSLTEQKHKYVPTAFCYYGYPLPEEQRFCKADSNGNAAGNTLEEAILQGFLELVERDSIAMWWYNRIRRPAVDLSTFDEPYFVDLQQFYQQQNRELWVLDVTADLGIPAFAGFSRRTVGTSERISIGFGAHLDPTIAILRALTEVSQVGLELDKIPDDKLDGESKDWMLNVTVENHPWLAPDPSVPMKTASDYPKRWSDDIHTDVMNCVKTAQTAGLEVMVLDQTRPDIGLNVVKVIIPGMRTFWTRFGQGRLYDIPVKLGWLDAPLAEEELNQTNIPF

SEQ ID NO: 8 - protein, 603 aa, Arthrospira platensis
MGSSHHHHHHGSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDIPTTENLYFQGAMGRTYPFAVSLNSTIQVSTTADGYAISPANTDPGQSIAPSMVTLPAITGMGDALAHLQAGTATLQQLTQTLSAREGVEAGEQLAATLQQMGDRGWLQYAVLPLAIAEPMVESAELDLNSPHWTQAKVSLSRFAYQRSHAGGMVLESPLSKFRVKLLDWRSSAILAQLAQPQPLGWVTPPPQIGAETAYQFLNLLWATGFLTVETEAPELKLWEFHNLLFHSRCRQGRHDYPTGDIAASLDIWDEFPVVKPPMSGHIVPLPQLSIDAIRQRDKTLTTAIEKRASIREYDENHPITIEQLGELLYRTARIKEIYTHDAEQAELLKAQFGEDFDWGELSRRPYPCGGAMYELEIYLAVRRCAGVKPGLYHYDPLNHQLAQIDAADADIQALLKDAHQSSGEQGMPQVLLMITARFGRLFRKYRSLAYALVLKHVGVLYQNLYLVATNMGLAPCALGAGDSDRFAQATGLDYVVESSVGEFMLGSL

SEQ ID NO: 9 - protein, 264 aa, Discosoma sp.
MHHHHHHMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKENLYFQGMCAYDGDYKDDDDK

SEQ ID NO: 10 - protein, 264 aa, Discosoma sp.
MHHHHHHMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKENLYFQGMAAYDGDYKDDDDK

SEQ ID NO: 11 - protein, 257 aa, Discosoma sp.
MDYKDDDDKMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDMCAYDGGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKHHHHHH

SEQ ID NO: 12 - protein, 257 aa, Discosoma sp.
MDYKDDDDKMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDMCAYDGGGHYDAEVKTTYKAKKPVQLPGAYNVNIKLDITSHNEDYTIVEQYERAEGRHSTGGMDELYKHHHHHH

SEQ ID NO: 13 - protein, 257 aa, Discosoma sp.
MDYKDDDDKMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVMCAYDGQLPGAYNVNIKLDITSHNEDYTIVEQYERAEGRHSTGGMDELYKHHHHHH

SEQ ID NO: 14 - protein, 257 aa, Discosoma sp.
MDYKDDDDKMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEMCAYDGDYTIVEQYERAEGRHSTGGMDELYKHHHHHH

SEQ ID NO: 15 - protein, 861 aa, Discosoma sp.
TCCCCTCTAGAAATAATTTTGTTTAACTTTAAGAAGGAGATATACATATGCACCACCATCACCATCATATGGTGAGCAAGGGCGAGGAGGATAACATGGCCATCATCAAGGAGTTCATGCGCTTCAAGGTGCACATGGAGGGCTCCGTGAACGGCCACGAGTTCGAGATCGAGGGCGAGGGCGAGGGCCGCCCCTACGAGGGCACCCAGACCGCCAAGCTGAAGGTGACCAAGGGTGGCCCCCTGCCCTTCGCCTGGGACATCCTGTCCCCTCAGTTCATGTACGGCTCCAAGGCCTACGTGAAGCACCCCGCCGACATCCCCGACTACTTGAAGCTGTCCTTCCCCGAGGGCTTCAAGTGGGAGCGCGTGATGAACTTCGAGGACGGCGGCGTGGTGACCGTGACCCAGGACTCCTCCCTGCAGGACGGCGAGTTCATCTACAAGGTGAAGCTGCGCGGCACCAACTTCCCCTCCGACGGCCCCGTAATGCAGAAGAAGACCATGGGCTGGGAGGCCTCCTCCGAGCGGATGTACCCCGAGGACGGCGCCCTGAAGGGCGAGATCAAGCAGAGGCTGAAGCTGAAGGACGGCGGCCACTACGACGCTGAGGTCAAGACCACCTACAAGGCCAAGAAGCCCGTGCAGCTGCCCGGCGCCTACAACGTCAACATCAAGTTGGACATCACCTCCCACAACGAGGACTACACCATCGTGGAACAGTACGAACGCGCCGAGGGCCGCCACTCCACCGGCGGCATGGACGAGCTGTACAAGGAAAATCTTTATTTTCAGGGAATGTGCGCCTACGACGGAGACTACAAAGACGACGACGACAAATAAGCGGCCGCACTCGAGCACC

SEQ ID NO: 16 - protein, 861 aa, Discosoma sp.
TCCCCTCTAGAAATAATTTTGTTTAACTTTAAGAAGGAGATATACATATGCACCACCATCACCATCATATGGTGAGCAAGGGCGAGGAGGATAACATGGCCATCATCAAGGAGTTCATGCGCTTCAAGGTGCACATGGAGGGCTCCGTGAACGGCCACGAGTTCGAGATCGAGGGCGAGGGCGAGGGCCGCCCCTACGAGGGCACCCAGACCGCCAAGCTGAAGGTGACCAAGGGTGGCCCCCTGCCCTTCGCCTGGGACATCCTGTCCCCTCAGTTCATGTACGGCTCCAAGGCCTACGTGAAGCACCCCGCCGACATCCCCGACTACTTGAAGCTGTCCTTCCCCGAGGGCTTCAAGTGGGAGCGCGTGATGAACTTCGAGGACGGCGGCGTGGTGACCGTGACCCAGGACTCCTCCCTGCAGGACGGCGAGTTCATCTACAAGGTGAAGCTGCGCGGCACCAACTTCCCCTCCGACGGCCCCGTAATGCAGAAGAAGACCATGGGCTGGGAGGCCTCCTCCGAGCGGATGTACCCCGAGGACGGCGCCCTGAAGGGCGAGATCAAGCAGAGGCTGAAGCTGAAGGACGGCGGCCACTACGACGCTGAGGTCAAGACCACCTACAAGGCCAAGAAGCCCGTGCAGCTGCCCGGCGCCTACAACGTCAACATCAAGTTGGACATCACCTCCCACAACGAGGACTACACCATCGTGGAACAGTACGAACGCGCCGAGGGCCGCCACTCCACCGGCGGCATGGACGAGCTGTACAAGGAAAATCTTTATTTTCAGGGAATGGCCGCCTACGACGGAGACTACAAAGACGACGACGACAAATAAGCGGCCGCACTCGAGCACC

SEQ ID NO: 17 - protein, 840 aa, Discosoma sp.
TCCCCTCTAGAAATAATTTTGTTTAACTTTAAGAAGGAGATATACATATGGACTACAAAGACGACGACGACAAAATGGTGAGCAAGGGCGAGGAGGATAACATGGCCATCATCAAGGAGTTCATGCGCTTCAAGGTGCACATGGAGGGCTCCGTGAACGGCCACGAGTTCGAGATCGAGGGCGAGGGCGAGGGCCGCCCCTACGAGGGCACCCAGACCGCCAAGCTGAAGGTGACCAAGGGTGGCCCCCTGCCCTTCGCCTGGGACATCCTGTCCCCTCAGTTCATGTACGGCTCCAAGGCCTACGTGAAGCACCCCGCCGACATCCCCGACTACTTGAAGCTGTCCTTCCCCGAGGGCTTCAAGTGGGAGCGCGTGATGAACTTCGAGGACGGCGGCGTGGTGACCGTGACCCAGGACTCCTCCCTGCAGGACGGCGAGTTCATCTACAAGGTGAAGCTGCGCGGCACCAACTTCCCCTCCGACATGTGCGCCTACGACGGAGGCCCCGTAATGCAGAAGAAGACCATGGGCTGGGAGGCCTCCTCCGAGCGGATGTACCCCGAGGACGGCGCCCTGAAGGGCGAGATCAAGCAGAGGCTGAAGCTGAAGGACGGCGGCCACTACGACGCTGAGGTCAAGACCACCTACAAGGCCAAGAAGCCCGTGCAGCTGCCCGGCGCCTACAACGTCAACATCAAGTTGGACATCACCTCCCACAACGAGGACTACACCATCGTGGAACAGTACGAACGCGCCGAGGGCCGCCACTCCACCGGCGGCATGGACGAGCTGTACAAGCACCACCATCACCATCATTAAGCGGCCGCACTCGAGCACC

SEQ ID NO: 18 - protein, 840 aa, Discosoma sp.
TCCCCTCTAGAAATAATTTTGTTTAACTTTAAGAAGGAGATATACATATGGACTACAAAGACGACGACGACAAAATGGTGAGCAAGGGCGAGGAGGATAACATGGCCATCATCAAGGAGTTCATGCGCTTCAAGGTGCACATGGAGGGCTCCGTGAACGGCCACGAGTTCGAGATCGAGGGCGAGGGCGAGGGCCGCCCCTACGAGGGCACCCAGACCGCCAAGCTGAAGGTGACCAAGGGTGGCCCCCTGCCCTTCGCCTGGGACATCCTGTCCCCTCAGTTCATGTACGGCTCCAAGGCCTACGTGAAGCACCCCGCCGACATCCCCGACTACTTGAAGCTGTCCTTCCCCGAGGGCTTCAAGTGGGAGCGCGTGATGAACTTCGAGGACGGCGGCGTGGTGACCGTGACCCAGGACTCCTCCCTGCAGGACGGCGAGTTCATCTACAAGGTGAAGCTGCGCGGCACCAACTTCCCCTCCGACGGCCCCGTAATGCAGAAGAAGACCATGGGCTGGGAGGCCTCCTCCGAGCGGATGTACCCCGAGGACGGCGCCCTGAAGGGCGAGATCAAGCAGAGGCTGAAGCTGAAGGACATGTGCGCCTACGACGGAGGCGGCCACTACGACGCTGAGGTCAAGACCACCTACAAGGCCAAGAAGCCCGTGCAGCTGCCCGGCGCCTACAACGTCAACATCAAGTTGGACATCACCTCCCACAACGAGGACTACACCATCGTGGAACAGTACGAACGCGCCGAGGGCCGCCACTCCACCGGCGGCATGGACGAGCTGTACAAGCACCACCATCACCATCATTAAGCGGCCGCACTCGAGCACC

SEQ ID NO: 19 - protein, 840 aa, Discosoma sp.
TCCCCTCTAGAAATAATTTTGTTTAACTTTAAGAAGGAGATATACATATGGACTACAAAGACGACGACGACAAAATGGTGAGCAAGGGCGAGGAGGATAACATGGCCATCATCAAGGAGTTCATGCGCTTCAAGGTGCACATGGAGGGCTCCGTGAACGGCCACGAGTTCGAGATCGAGGGCGAGGGCGAGGGCCGCCCCTACGAGGGCACCCAGACCGCCAAGCTGAAGGTGACCAAGGGTGGCCCCCTGCCCTTCGCCTGGGACATCCTGTCCCCTCAGTTCATGTACGGCTCCAAGGCCTACGTGAAGCACCCCGCCGACATCCCCGACTACTTGAAGCTGTCCTTCCCCGAGGGCTTCAAGTGGGAGCGCGTGATGAACTTCGAGGACGGCGGCGTGGTGACCGTGACCCAGGACTCCTCCCTGCAGGACGGCGAGTTCATCTACAAGGTGAAGCTGCGCGGCACCAACTTCCCCTCCGACGGCCCCGTAATGCAGAAGAAGACCATGGGCTGGGAGGCCTCCTCCGAGCGGATGTACCCCGAGGACGGCGCCCTGAAGGGCGAGATCAAGCAGAGGCTGAAGCTGAAGGACGGCGGCCACTACGACGCTGAGGTCAAGACCACCTACAAGGCCAAGAAGCCCGTGATGTGCGCCTACGACGGACAGCTGCCCGGCGCCTACAACGTCAACATCAAGTTGGACATCACCTCCCACAACGAGGACTACACCATCGTGGAACAGTACGAACGCGCCGAGGGCCGCCACTCCACCGGCGGCATGGACGAGCTGTACAAGCACCACCATCACCATCATTAAGCGGCCGCACTCGAGCACC

SEQ ID NO: 20 - protein, 840 aa, Discosoma sp.
TCCCCTCTAGAAATAATTTTGTTTAACTTTAAGAAGGAGATATACATATGGACTACAAAGACGACGACGACAAAATGGTGAGCAAGGGCGAGGAGGATAACATGGCCATCATCAAGGAGTTCATGCGCTTCAAGGTGCACATGGAGGGCTCCGTGAACGGCCACGAGTTCGAGATCGAGGGCGAGGGCGAGGGCCGCCCCTACGAGGGCACCCAGACCGCCAAGCTGAAGGTGACCAAGGGTGGCCCCCTGCCCTTCGCCTGGGACATCCTGTCCCCTCAGTTCATGTACGGCTCCAAGGCCTACGTGAAGCACCCCGCCGACATCCCCGACTACTTGAAGCTGTCCTTCCCCGAGGGCTTCAAGTGGGAGCGCGTGATGAACTTCGAGGACGGCGGCGTGGTGACCGTGACCCAGGACTCCTCCCTGCAGGACGGCGAGTTCATCTACAAGGTGAAGCTGCGCGGCACCAACTTCCCCTCCGACGGCCCCGTAATGCAGAAGAAGACCATGGGCTGGGAGGCCTCCTCCGAGCGGATGTACCCCGAGGACGGCGCCCTGAAGGGCGAGATCAAGCAGAGGCTGAAGCTGAAGGACGGCGGCCACTACGACGCTGAGGTCAAGACCACCTACAAGGCCAAGAAGCCCGTGCAGCTGCCCGGCGCCTACAACGTCAACATCAAGTTGGACATCACCTCCCACAACGAGATGTGCGCCTACGACGGAGACTACACCATCGTGGAACAGTACGAACGCGCCGAGGGCCGCCACTCCACCGGCGGCATGGACGAGCTGTACAAGCACCACCATCACCATCATTAAGCGGCCGCACTCGAGCACC

SEQ ID NO: 21 - protein, 86 aa, Escherichia coli; identical to SEQ ID NO: 1

SEQ ID NO: 22 - protein, 86 aa, Escherichia coli; identical to SEQ ID NO: 2

SEQ ID NO: 23 - protein, 86 aa, Escherichia coli; identical to SEQ ID NO: 3

SEQ ID NO: 24 - protein, 86 aa, Escherichia coli
MDYKDDDDKMTKQEKTALNMARFIRSQTLTLLEKLNELGGGGMAAYDGDEQADICESLHDHADELYRSCLARFGDDGENLHHHHHH

SEQ ID NO: 25 - protein, 86 aa, Escherichia coli; identical to SEQ ID NO: 4

SEQ ID NO: 26 - protein, 327 aa, Escherichia coli
TCCCCTCTAGAAATAATTTTGTTTAACTTTAAGAAGGAGATATACATATGGACTACAAAGACGACGACGACAAAATGACCAAACAGGAAAAAACCGCCCTTAACATGGCCCGCTTTATCAGAAGCCAGACATTAACGCTTCTGGAGAAACTCAACGAGCTGATGTGCGCCTACGACGGAGGGGGCGGTGGCGATGAACAGGCAGACATCTGTGAATCGCTTCACGACCACGCTGATGAGCTTTACCGCAGCTGCCTCGCGCGTTTCGGTGATGACGGTGAAAACCTCCACCACCATCACCATCATTAAGCGGCCGCACTCGAGCACC

SEQ ID NO: 27 - protein, 327 aa, Escherichia coli
TCCCCTCTAGAAATAATTTTGTTTAACTTTAAGAAGGAGATATACATATGGACTACAAAGACGACGACGACAAAATGACCAAACAGGAAAAAACCGCCCTTAACATGGCCCGCTTTATCAGAAGCCAGACATTAACGCTTCTGGAGAAACTCAACGAGCTGGGCGGGATGTGCGCCTACGACGGAGGTGGCGATGAACAGGCAGACATCTGTGAATCGCTTCACGACCACGCTGATGAGCTTTACCGCAGCTGCCTCGCGCGTTTCGGTGATGACGGTGAAAACCTCCACCACCATCACCATCATTAAGCGGCCGCACTCGAGCACC

SEQ ID NO: 28 - protein, 327 aa, Escherichia coli
TCCCCTCTAGAAATAATTTTGTTTAACTTTAAGAAGGAGATATACATATGGACTACAAAGACGACGACGACAAAATGACCAAACAGGAAAAAACCGCCCTTAACATGGCCCGCTTTATCAGAAGCCAGACATTAACGCTTCTGGAGAAACTCAACGAGCTGGGCGGGGGGGGAATGTGCGCCTACGACGGAGATGAACAGGCAGACATCTGTGAATCGCTTCACGACCACGCTGATGAGCTTTACCGCAGCTGCCTCGCGCGTTTCGGTGATGACGGTGAAAACCTCCACCACCATCACCATCATTAAGCGGCCGCACTCGAGCACC

SEQ ID NO: 29 - protein, 327 aa, Escherichia coli
TCCCCTCTAGAAATAATTTTGTTTAACTTTAAGAAGGAGATATACATATGGACTACAAAGACGACGACGACAAAATGACCAAACAGGAAAAAACCGCCCTTAACATGGCCCGCTTTATCAGAAGCCAGACATTAACGCTTCTGGAGAAACTCAACGAGCTGGGCGGAGGTGGAATGGCCGCCTACGACGGAGATGAACAGGCAGACATCTGTGAATCGCTTCACGACCACGCTGATGAGCTTTACCGCAGCTGCCTCGCGCGTTTCGGTGATGACGGTGAAAACCTCCACCACCATCACCATCATTAAGCGGCCGCACTCGAGCACC

SEQ ID NO: 30 - protein, 327 aa, Escherichia coli
TCCCCTCTAGAAATAATTTTGTTTAACTTTAAGAAGGAGATATACATATGGACTACAAAGACGACGACGACAAAATGACCAAACAGGAAAAAACCGCCCTTAACATGGCCCGCTTTATCAGAAGCCAGACATTAACGCTTCTGGAGAAACTCAACGAGCTGGGCGGGGGTGGAATGTGCGGTGGGGGAGGAGATGAACAGGCAGACATCTGTGAATCGCTTCACGACCACGCTGATGAGCTTTACCGCAGCTGCCTCGCGCGTTTCGGTGATGACGGTGAAAACCTCCACCACCATCACCATCATTAAGCGGCCGCACTCGAGCACC

SEQ ID NO: 31 - protein, 6 aa, synthetic construct
ICAYDG

SEQ ID NO: 32 - protein, 5 aa, synthetic construct
CAYDG

SEQ ID NO: 33 - protein, 5 aa, synthetic construct
CAYDG

SEQ ID NO: 34 - protein, 5 aa, synthetic construct
CAYDG

SEQ ID NO: 35 - protein, 5 aa, synthetic construct
CAYDG

SEQ ID NO: 36 - protein, 5 aa, synthetic construct
CAYDG

SEQ ID NO: 37 - protein, 5 aa, synthetic construct
CAYDG

SEQ ID NO: 38 - protein, 5 aa, synthetic construct
CAYDG

SEQ ID NO: 39 - protein, 5 aa, synthetic construct
CAYDG

SEQ ID NO: 40 - protein, 5 aa, synthetic construct
CAYDG

SEQ ID NO: 41 - protein, 7 aa, synthetic construct
ICIAYDG

SEQ ID NO: 42 - protein, 4 aa, synthetic construct
AYDG

SEQ ID NO: 43 - protein, 4 aa, synthetic construct
AYDG

SEQ ID NO: 44 - protein, 4 aa, synthetic construct
AYDG

SEQ ID NO: 45 - protein, 4 aa, synthetic construct
AYDG

SEQ ID NO: 46 - protein, 4 aa, synthetic construct
AYDG

SEQ ID NO: 47 - protein, 4 aa, synthetic construct
AYDG

SEQ ID NO: 48 - protein, 4 aa, synthetic construct
AYDG

SEQ ID NO: 49 - protein, 6 aa, Discosoma sp.
MCAYDG

SEQ ID NO: 50 - protein, 6 aa, Discosoma sp.
MAAYDG

SEQ ID NO: 51 - protein, 6 aa, Discosoma sp.
MCAYDG

SEQ ID NO: 52 - protein, 6 aa, Discosoma sp.
MCAYDG

SEQ ID NO: 53 - protein, 6 aa, Discosoma sp.
MCAYDG

SEQ ID NO: 54 - protein, 6 aa, Discosoma sp.
MCAYDG

SEQ ID NO: 55 - protein, 10 aa, Escherichia coli
MCAYDGGGGG

SEQ ID NO: 56 - protein, 10 aa, Escherichia coli
GGMCAYDGGG

SEQ ID NO: 57 - protein, 10 aa, Escherichia coli
GGGGMCAYDG

SEQ ID NO: 58 - protein, 10 aa, Escherichia coli
GGGGMCGGGG

SEQ ID NO: 59 - protein, 10 aa, Escherichia coli
GGGGMAAYDG